(19) United States
(12) Patent Application Publication
(10) Pub. No.: US 2020/0339987 A1
(43) Pub. Date: Oct. 29, 2020

(54) ASSAYS AND METHODS FOR DETERMINING EXPRESSION OF THE LECT2 GENE

(71) Applicant: ALNYLAM PHARMACEUTICALS, INC., Cambridge, MA (US)

(72) Inventors: Gregory Hinkle, Plymouth, MA (US); Ho-Chou Tu, Boston, MA (US); Alfica Sehgal, Medford, MA (US)

(21) Appl. No.: 16/762,546

(22) PCT Filed: Nov. 8, 2018
(86) PCT No.: PCT/US2018/059805

§ 371 Date: May 8, 2020

Related U.S. Application Data
(60) Provisional application No. 62/583,749, filed on Nov. 9, 2017.

(51) Int. Cl. C12N 15/113; C12Q 1/6883; C12N 15/10; A61K 9/00; A61K 9/10

(52) U.S. Cl. CPC C12N 15/113; C12N 2320/35; C12N 15/1003; A61K 9/0019; A61K 9/10; C12Q 2600/158; C12Q 2600/106; C12N 2310/11; C12N 2310/321; C12N 2310/315; C12N 2310/322; C12N 2310/3231; C12N 2310/314; C12N 2310/352; C12N 2320/32; C12N 2310/3341; C12Q 1/6883

(57) ABSTRACT

The invention relates to methods and assays for determining the activity or expression of LECT2 gene in a subject.

### CROSS-REFERENCE TO RELATED APPLICATIONS

This application claims the benefit of U.S. Provisional Application No. 62/583,749, filed Nov. 9, 2017, the contents of which are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Nov. 7, 2018, is named A2038-7227WO_SL.txt and is 143,506 bytes in size.

### FIELD OF THE INVENTION

The disclosure relates to methods for determining expression of the LECT2 gene.

### BACKGROUND

Amyloidosis is a group of diseases characterized by deposition of insoluble fibrous protein aggregates, called amyloids, in organs or tissues. Amyloids can form from mutant or wild type proteins. One system of nomenclature for amyloid diseases uses an abbreviation for the protein that forms amyloid deposits, preceded by the letter “A.” Thus, for example, ALECT2 is the abbreviation for an amyloidosis involving deposit of amyloids formed from leukocyte cell derived chemotactic factor-2 (ALECT2).

LECT2 amyloidosis (ALECT2) is one of the most recently discovered types of amyloidosis. LECT2 amyloidosis has been observed in individuals with renal or hepatic amyloidosis. This form of amyloidosis can present with renal insufficiency or nephrotic syndrome or with liver involvement (e.g., hepatitis, e.g., chronic hepatitis). It may be particularly prevalent in Mexican Americans and/or individuals who are homozygous for the G allele encoding valine at position 40 in the mature LECT2 protein (or at position 58 in the unprocessed protein). Diagnoses and treatments for LECT2 amyloidosis are limited, and new methods are needed.

### SUMMARY

Provided herein are methods and compositions for determining activity or expression of a LECT2 gene. Such methods and compositions are useful for evaluating or treating a subject for a LECT2-associated disorder (e.g., an ALECT2-associated disorder). Also provided herein are methods and compositions for determining activity of a nucleic acid agent (e.g., a double-stranded ribonucleic acid (dsRNA) or an antisense polynucleotide agent) targeting a LECT2 RNA. Such methods and compositions can be used to determine efficacy of the nucleic acid agent or to monitor a therapy comprising the nucleic acid agent.

In one aspect, the disclosure features a method of determining an activity or expression of a LECT2 gene in a subject, comprising: i) acquiring a bodily fluid sample from the subject; and ii) detecting the level of an LECT2 mRNA in the sample; wherein an increase in the level of the LECT2 mRNA, as compared to a reference LECT2 mRNA level, is indicative of an increase in the activity or expression of the LECT2 gene at a site distal from the sample, thereby determining the activity or expression of the LECT2 gene in the subject.

In some embodiments, the increase in the level of the LECT2 mRNA in the sample is indicative of the increase in the activity or expression of the LECT2 gene in a cell expressing the LECT2 mRNA. In some embodiments, the increase in the level of the LECT2 mRNA in a urine or blood sample is indicative of the increase in the activity or expression of the LECT2 gene in a liver cell. In some embodiments, the increase in the activity or expression of the LECT2 gene is indicative of having, or an increased risk of having, a LECT2-associated disorder in the subject. In some embodiments, the method further comprises administering to the subject a double-stranded ribonucleic acid (dsRNA) or antisense polynucleotide agent that inhibits expression of the LECT2 mRNA.

In another aspect, the disclosure features a method of evaluating a subject for a LECT2-associated disorder, comprising: acquiring knowledge of the level of an LECT2 mRNA in a bodily fluid sample from the subject, wherein an increase in the level of the LECT2 mRNA in the sample, as compared to a reference LECT2 mRNA level, is indicative of having, or an increased risk of having, the LECT2-associated disorder in the subject, thereby evaluating the subject for the LECT2-associated disorder.

In some embodiments, acquiring knowledge of the level of the LECT2 mRNA comprises detecting the level of the LECT2 mRNA in the sample. In some embodiments, the increase in the level of the LECT2 mRNA in the sample is indicative of an increase in an activity or expression of a LECT2 gene at a site distal from the sample. In some embodiments, the increase in the level of the LECT2 mRNA in a urine or blood sample is indicative of the increase in the activity or expression of the LECT2 gene in a liver cell. In some embodiments, the method further comprises administering to the subject a dsRNA or antisense polynucleotide agent that inhibits expression of the LECT2 mRNA.

In another aspect, the disclosure features a method of identifying a subject for a therapy for a LECT2-associated disorder, the method comprising: i) acquiring a bodily fluid sample from the subject; and ii) detecting the level of an LECT2 mRNA in the sample; wherein an increase in the level of the LECT2 mRNA, as compared to a reference LECT2 mRNA level, is indicative of a subject suitable for the therapy, wherein the therapy comprises a dsRNA or antisense polynucleotide agent inhibits expression of the LECT2 mRNA, thereby identifying the subject for the therapy for the LECT2-associated disorder.

In some embodiments, the increase in the level of the LECT2 mRNA in the sample is indicative of an increase in an activity or expression of a LECT2 gene at a site distal from the sample. In some embodiments, the increase in the level of the LECT2 mRNA in a urine or blood sample is indicative of the increase in the activity or expression of a LECT2 gene in a liver cell. In some embodiments, the increase in the activity or expression of the LECT2 gene is indicative of having, or an increased risk of having, the LECT2-associated disorder in the subject. In some embodiments, the method further comprises administering to the subject the dsRNA or the antisense polynucleotide agent that inhibits expression of the LECT2 mRNA.

In another aspect, the disclosure features a method of treating a LECT2-related disorder in a subject, comprising: responsive to the determination of an increase in the level of an LECT2 mRNA in a bodily fluid sample from the subject, as compared to a reference LECT2 mRNA level, administering to the subject a dsRNA or antisense polynucleotide agent that inhibits expression of the LECT2 mRNA, thereby treating the LECT2-related disorder in the subject.

In some embodiments, the method further comprises detecting the level of the LECT2 mRNA in the sample. In some embodiments, the method further comprises acquiring the sample from the subject. In some embodiments, the increase in the level of the LECT2 mRNA in the sample is indicative of an increase in an activity or expression of a LECT2 gene at a site distal from the sample. In some embodiments, the increase in the level of the LECT2 mRNA in a urine or blood sample is indicative of the increase in the activity or expression of a LECT2 gene in a liver cell.

In another aspect, the disclosure features a method of reducing an activity or expression of a LECT2 gene, comprising: i) acquiring knowledge of the level of an LECT2 mRNA encoded by the LECT2 gene in a body fluid sample, wherein an increase in the level of the LECT2 mRNA, as compared to a reference LECT2 mRNA level, is indicative of an increase in the activity or expression of the LECT2 gene in a cell at a site distal to the sample; and ii) contact the cell with a dsRNA or antisense polynucleotide agent that inhibits expression of the LECT2 mRNA, thereby reducing the activity or expression of the LECT2 gene.

In some embodiments, the method further comprises detecting the level of the LECT2 mRNA in the sample. In some embodiments, the increase in the level of the LECT2 mRNA in a urine or blood sample is indicative of the increase in the activity or expression of a LECT2 gene in a liver cell. In some embodiments, the method further comprises acquiring the sample from a subject. In some embodiments, the increase in the activity or expression of the LECT2 gene is indicative of having, or an increased risk of having, a LECT2-associated disorder in the subject.

In another aspect, the disclosure features a method of determining an activity of a dsRNA or antisense polynucleotide agent in a subject, the method comprising: i) acquiring a bodily fluid sample from a subject who has been administered the dsRNA or antisense polynucleotide agent, wherein the dsRNA or antisense polynucleotide agent inhibits expression of an LECT2 mRNA; and ii) detecting the level of the LECT2 mRNA, or a cleavage product thereof, in the sample, wherein a decrease in the level of the LECT2 mRNA, or an increase in the level of the cleavage product, as compared to a reference level of the LECT2 mRNA, or the cleavage product thereof, is indicative that the dsRNA or antisense polynucleotide agent is active in the subject.

In some embodiments, the decrease in the level of the LECT2 mRNA, or the increase in the level of the cleavage product, in the sample, is indicative of an inhibition of expression of the LECT2 mRNA at a site distal from the subject. In some embodiments, the decrease in the level of the LECT2 mRNA, or the increase in the level of the cleavage product, in a urine or blood sample, is indicative of an inhibition of expression of the LECT2 mRNA in a liver cell.

In some embodiments, the subject has been administered a therapy for a LECT2-associated disorder comprising the dsRNA or antisense polynucleotide agent.

In some embodiments, the method comprises responsive to the determination that the dsRNA or antisense polynucleotide agent is active in the subject, adjusting the dosage of the dsRNA or antisense polynucleotide agent. In some embodiments, the dose is decreased, the interval between doses is increased, or both. In some embodiments, responsive to the determination that the dsRNA or antisense polynucleotide agent is active in the subject, administration of the dsRNA or antisense polynucleotide agent to the subject is continued.

In some embodiments, an increased or unchanged level of the LECT2 mRNA, or an unchanged or decreased level of the cleavage product, is indicative that the dsRNA or antisense polynucleotide agent is inactive in the subject. In some embodiments, the method comprises responsive to the determination that the dsRNA or antisense polynucleotide agent is inactive in the subject, adjusting the dosage of the dsRNA or antisense polynucleotide agent. In some embodiments, the dose is increased, the interval between doses is decreased, or both. In some embodiments, responsive to the determination that the dsRNA or antisense polynucleotide agent is inactive in the subject, administration of the dsRNA or antisense polynucleotide agent to the subject is discontinued. In some embodiments, the method comprises responsive to the determination that the dsRNA or antisense polynucleotide agent is inactive in the subject, administering to the subject an alternative therapy for a LECT2-associated disorder.

In another aspect, the disclosure features a dsRNA or antisense polynucleotide agent for use in treating a LECT2-related disorder in a subject, wherein the dsRNA or antisense polynucleotide agent inhibits expression of a LECT2 mRNA, and wherein the dsRNA or antisense polynucleotide agent is used responsive to the determination of an increase in the level of an LECT2 mRNA in a bodily fluid sample from the subject, as compared to a reference LECT2 mRNA level. In some embodiments, use of the dsRNA or antisense polynucleotide agent further comprises detecting the level of the LECT2 mRNA in the sample. In some embodiments, use of the dsRNA or antisense polynucleotide agent further comprises acquiring the sample from the subject. In some embodiments, the increase in the level of the LECT2 mRNA in the sample is indicative of an increase in an activity or expression of a LECT2 gene at a site distal from the sample. In some embodiments, the increase in the level of the LECT2 mRNA in a urine or blood sample is indicative of the increase in the activity or expression of a LECT2 gene in a liver cell.

In another aspect, the disclosure features an assay for determining an activity or expression of a LECT2 gene in liver of a subject, comprising: i) acquiring a urine or blood sample from the subject; ii) contacting the sample with a probe that hybridizes to a LECT2 mRNA encoded by the LECT2 gene; iii) detecting the level of the LECT2 mRNA in the sample; wherein an increase in the level of the LECT2 mRNA, as compared to a reference LECT2 mRNA level, is indicative of an increase in the activity or expression of the LECT2 gene in the liver.

In another aspect, the disclosure features an assay for monitoring efficacy of a dsRNA or antisense polynucleotide agent for treating a LECT2 amyloidosis (ALECT2) in a subject, comprising: i) acquiring a urine or blood sample from the subject; ii) contacting the sample with a probe that hybridizes to a LECT2 mRNA or a cleavage product thereof; and iii) detecting the level of the LECT2 mRNA or the cleavage product in the sample; wherein a decrease in the level of the LECT2 mRNA, or an increase in the level of the cleavage product, as compared to a reference level of the LECT2 mRNA or cleavage product, is indicative that the dsRNA or antisense polynucleotide agent is efficacious in the treatment of the ALECT2.

Other features and embodiments of the methods and assays described herein can include one or more of the following:

In some embodiments, the sample is chosen from a urine sample, a blood sample, a synovial fluid sample, a cerebrospinal fluid (CSF) sample, an amniotic fluid sample, a saliva sample, a breast milk sample, a bronchoalveolar lavage fluid sample, or a malignant ascites sample. In some embodiments, the sample is a urine sample. In some embodiments, the blood sample is a serum sample or a plasma sample. In some embodiments, the sample is a serum sample. In some embodiments, the sample comprises circulating extracellular RNA. In some embodiments, the sample comprises exosomes. In some embodiments, the sample does not comprise exosomes.

In some embodiments, the reference level of the LECT2 mRNA is the level of the LECT2 mRNA in a bodily fluid sample from a healthy subject. In some embodiments, the reference level of the LECT2 mRNA is the level of the LECT2 mRNA in a bodily sample from a subject who does not have a LECT2-related disorder or a symptom of thereof. In some embodiments, the reference level of the LECT2 mRNA is the level of the LECT2 mRNA in a bodily sample from a subject who has a chronic kidney disease (CKD). In some embodiments, the reference level of the LECT2 mRNA is the level of the LECT2 mRNA in a bodily sample from a subject who has amyloid light-chain (AL) amyloidosis. In some embodiments, the reference level of the LECT2 mRNA is the level of the LECT2 mRNA in a bodily sample from the subject prior to administration of the dsRNA or antisense polynucleotide agent. In some embodiments, wherein the reference level of the LECT2 mRNA is the level of the LECT2 mRNA in a bodily fluid sample from the subject within 1 hour, 2 hours, 6 hours, 12 hours, 24 hours, 48 hours, 72 hours, or 96 hours after administration of the dsRNA or antisense polynucleotide agent. In some embodiments, the reference level of the LECT2 mRNA is the average level of the LECT2 mRNA in bodily fluid samples from a plurality of subjects. In some embodiments, the reference level of the cleavage product is the level of the cleavage product in a bodily sample from a subject who has not been administered the dsRNA or antisense polynucleotide agent. In some embodiments, the reference level of the cleavage product is the level of the cleavage product in a sample from the subject within 1 hour, 2 hours, 6 hours, 12 hours, 24 hours, 48 hours, 72 hours, or 96 hours after administration of the dsRNA or antisense polynucleotide agent. In some embodiments, the reference level of the cleavage product is the average level of the cleavage product in bodily samples from a plurality of subjects.

In some embodiments, the method comprises centrifuging the sample prior to detecting the level of the LECT2 mRNA, or the cleavage product thereof. In some embodiments, the sample is centrifuged at 100,000 g to 300,000 g, e.g., 150,000 g to 250,000 g, 100,000 g to 150,000 g, 150,000 g to 200,000 g, 200,000 g to 250,000 g, 250,000 g to 300,000 g, 100,000 to 200,000 g, or 200,000 g to 300,000 g. In some embodiments, the sample is centrifuged at 180,000 g to 220,000 g, e.g., about 200,000 g.

In some embodiments, the method comprises producing a cDNA complementary to the LECT2 mRNA or the cleavage product thereof. In some embodiments, the method further comprises amplifying the cDNA. In some embodiments, the method comprises detecting the level of the LECT2 mRNA, or the cleavage product thereof, using 5′ RACE, hybridization, polymerase chain reaction (PCR), quantitative PCR (qPCR), branched DNA (bDNA), or reverse transcription-PCR (RT-PCR).

In some embodiments, the method comprises contacting the sample with a reagent for isolating RNA. In some embodiments, the reagent for isolating RNA comprises TRIzol™ reagent, chloroform, phenol/chloroform, or a combination thereof. In some embodiments, the method comprises contacting the sample with a reagent for enhancing RNA precipitation. In some embodiments, the reagent for enhancing RNA precipitation comprises glycogen or polyethylene glycol (PEG). In some embodiments, the method comprises contacting the sample with an RNase inhibitor. In some embodiments, the RNase inhibitor comprises EDTA. In some embodiments, the method comprises contacting the sample with a reagent for increasing RNA yield. In some embodiments, the reagent for increasing RNA yield comprises lithium chloride (LiCl). In some embodiments, the reagent for increasing RNA yield comprises sodium acetate. In some embodiments, the reagent for increasing RNA yield is used at a final concentration of 1 M. In some embodiments, the reagent for increasing RNA yield is contacted with the sample prior to precipitation of the RNA.

In some embodiments, the method comprises contacting the isolated RNA with a primer. In some embodiments, the primer comprises a nucleotide sequence described herein. In some embodiments, the primer comprises an oligo-dT sequence. In some embodiments, the primer is suitable for PCR. In some embodiments, the primer is suitable for 5′ RACE.

In some embodiments, the method comprises normalizing the level of the LECT2 mRNA or the cleavage product thereof. In some embodiments, the level of the LECT2 mRNA or the cleavage product thereof is normalized to the level of 18s RNA, GAPDH mRNA, or β-actin mRNA in the same sample. In some embodiments, the level of the LECT2 mRNA or the cleavage product thereof is normalized to the level of an mRNA encoding a liver protein in the same sample. In some embodiments, the liver protein is factor VII, albumin, or alpha antitrypsin (AAT). In some embodiments, the level of the LECT2 mRNA or the cleavage product thereof is detected without purification (e.g., affinity purification) of exosomes from the sample.

In some embodiments, the dsRNA or antisense polynucleotide agent targets a LECT2 mRNA expressed in liver, kidney, brain, spinal cord, choroid plexus, peripheral neurons or nerve, muscle, endothelial cells, heart, immune cells, skin, eye, pancreas, lung, stomach, small or large intestines, colon, adrenal gland, tumors, cancer lesions, or spleen. In some embodiments, the dsRNA or antisense polynucleotide agent targets a LECT2 mRNA expressed in liver.

In some embodiments, the dsRNA or antisense polynucleotide agent reduces LECT2 mRNA expression in a cell in the subject. In some embodiments, the cell is a liver cell or a hepatocyte.

In some embodiments, the dsRNA or antisense polynucleotide agent reduces LECT2 mRNA expression in the subject by at least 20%. In some embodiments, the dsRNA or antisense polynucleotide agent reduces LECT2 mRNA expression in the subject by at least 30%. In some embodiments, the dsRNA or antisense polynucleotide agent reduces LECT2 mRNA expression in the subject by at least 40%. In some embodiments, the dsRNA or antisense polynucleotide agent reduces LECT2 mRNA expression in the subject by at least 50%.

In some embodiments, the dsRNA or antisense polynucleotide agent reduces the level of LECT2 mRNA in urine or blood, by at least 10%, 20%, 30%, 40%, or 50%, within 1, 2, 3, 7, 14, 21, or 28 days after administration of the dsRNA or antisense polynucleotide agent. In some embodiments, the dsRNA or antisense polynucleotide agent reduces the level of LECT2 mRNA in a liver, by at least 10%, 20%, 30%, 40%, or 50%, within 1, 2, 3, 7, 14, 21, or 28 days after administration of the dsRNA or antisense polynucleotide agent.

In some embodiments, the dsRNA or antisense polynucleotide agent reduces LECT2 deposition in the kidney of the subject. In some embodiments, the dsRNA comprises a sense strand that is 15-30 base pairs in length and an antisense strand that is 15-30 base pairs in length, wherein the antisense strand is complementary to at least 15 contiguous nucleotides of SEQ ID NO: 1 or a nucleotide sequence having an A to G substitution at nucleotide position 373 of SEQ ID NO: 1.

In some embodiments, the dsRNA comprises a sense strand and an antisense strand, the antisense strand comprising a region of complementarity to a LECT2 RNA transcript, wherein the antisense strand comprises at least 15 contiguous nucleotides differing by no more than 3 nucleotides from one of the antisense sequences listed in Tables 2 and 3.

In some embodiments, the dsRNA comprises a duplex region that is 15-30 nucleotide pairs in length. In some embodiments, the duplex region is 17-23 nucleotide pairs in length. In some embodiments, the duplex region is 19-21 nucleotide pairs in length. In some embodiments, the duplex region is 21-23 nucleotide pairs in length. In some embodiments, the dsRNA comprises a region of complementarity that is at least 17 nucleotides in length. In some embodiments, the region of complementarity is 19 nucleotides in length. In some embodiments, the region of complementarity is between 19 and 21 nucleotides in length. In some embodiments, at least one strand of the dsRNA comprises a 3′ overhang of at least 1 nucleotide. In some embodiments, at least one strand of the dsRNA comprises a 3′ overhang of at least 2 nucleotides.

In some embodiments, the dsRNA comprises at least one modified nucleotide. In some embodiments, the at least one modified nucleotide is chosen from a 2′-O-methyl modified nucleotide, a nucleotide comprising a 5′-phosphorothioate group, or a terminal nucleotide linked to a cholesteryl derivative or dodecanoic acid bisdecylamide group. In some embodiments, the at least one modified nucleotide is chosen from a 2′-deoxy-2′-fluoro modified nucleotide, a 2′-deoxy-modified nucleotide, a locked nucleic acid (LNA), an acyclic nucleotide, an abasic nucleotide, 2′-amino-modified nucleotide, 2′-alkyl-modified nucleotide, morpholino nucleotide, a phosphoramidate, or a non-natural base comprising nucleotide. In some embodiments, the at least one modified nucleotide comprises a modification selected from the group consisting of locked nucleic acid (LNA), an acyclic nucleotide, hexitol or hexose nucleic acid (HNA), cyclohexene nucleic acid (CeNA), 2′-methoxyethyl, 2′-O-alkyl, 2′-O-allyl, 2′-C-allyl, 2′-fluoro, 2′-O-methyl, 2′-deoxy, 2′-hydroxyl, and combinations thereof. In some embodiments, the at least one modified nucleotide comprises 2′-O-methyl, 2′-fluoro, or both.

In some embodiments, the sense strand is conjugated to at least one ligand. In some embodiments, the ligand is attached to the 3′ end of the sense strand. In some embodiments, the ligand comprises a carbohydrate. In some embodiments, the ligand is a GalNAc ligand.

In some embodiments, the ligand is

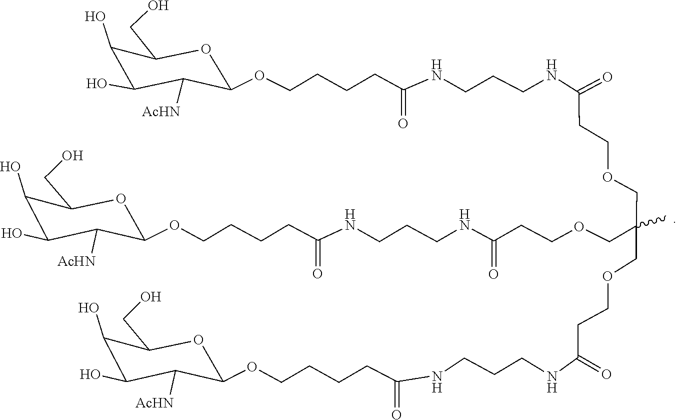 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In some embodiments, the ligand is attached via a linker. In some embodiments, the linker is a bivalent or trivalent branched linker.

In some embodiments, the ligand and linker are as shown in Formula XXIV:

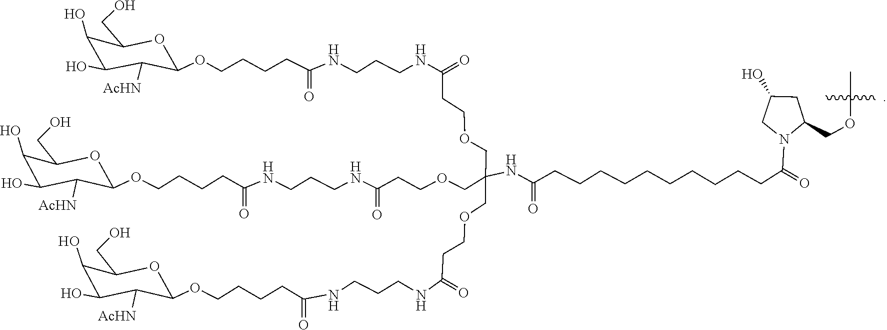 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

In some embodiments, the ligand targets the dsRNA to hepatocytes.

In some embodiments, the dsRNA comprises a region of complementarity that comprises an antisense sequence selected from the antisense sequences disclosed in Tables 2 and 3. In some embodiments, the dsRNA comprises a sense strand comprising a sense sequence selected from the sense sequences disclosed in Tables 2 and 3, and an antisense strand comprising an antisense sequence selected from the antisense sequences disclosed in Tables 2 and 3. In some embodiments, the dsRNA comprises a region of complementarity that consists of an antisense sequence selected from the antisense sequences disclosed in Tables 2 and 3. In some embodiments, the dsRNA comprises a sense strand consisting of a sense sequence selected from the sense sequences disclosed in Tables 2 and 3, and an antisense strand consisting of an antisense sequence selected from the antisense sequences disclosed in Tables 2 and 3.

In some embodiments, the antisense polynucleotide agent comprises about 4 to about 50 contiguous nucleotides, wherein at least one of the contiguous nucleotides is a modified nucleotide, and wherein the nucleotide sequence of the agent is about 80% complementary over its entire length to the equivalent region of the nucleotide sequence of SEQ ID NO: 1. In some embodiments, the equivalent region is one of the target regions of SEQ ID NO: 1 provided in Table 3.

In some embodiments, the antisense polynucleotide agent comprises at least 8 contiguous nucleotides differing by no more than 3 nucleotides from any one of the nucleotide sequences listed in Table 3.

In some embodiments, the antisense polynucleotide agent is 10 to 40 nucleotides in length. In some embodiments, the antisense polynucleotide agent is 10 to 30 nucleotides in length. In some embodiments, the antisense polynucleotide agent is 18 to 30 nucleotides in length. In some embodiments, the antisense polynucleotide agent is 10 to 24 nucleotides in length. In some embodiments, the antisense polynucleotide agent is 18 to 24 nucleotides in length. In some embodiments, the antisense polynucleotide agent is 14 or 20 nucleotides in length.

In some embodiments, substantially all of the nucleotides of the antisense polynucleotide agent are modified nucleotides. In some embodiments, all of the nucleotides of the antisense polynucleotide agent are modified nucleotides.

In some embodiments, the modified nucleotide comprises a modified sugar moiety selected from the group consisting of: a 2′-O-methoxyethyl modified sugar moiety, a 2′-methoxy modified sugar moiety, a 2′-O-alkyl modified sugar moiety, and a bicyclic sugar moiety. In some embodiments, the bicyclic sugar moiety has a (—CH2-)n group forming a bridge between the 2′ oxygen and the 4′ carbon atoms of the sugar ring, wherein n is 1 or 2.

In some embodiments, the modified nucleotide is a 5-methylcytosine. In some embodiments, the modified nucleotide comprises a modified internucleoside linkage. In some embodiments, the modified internucleoside linkage is a phosphorothioate internucleoside linkage.

In some embodiments, the antisense polynucleotide agent comprises a plurality of 2′-deoxynucleotides flanked on each side by at least one nucleotide having a modified sugar moiety. In some embodiments, the antisense polynucleotide agent is a gapmer comprising a gap segment comprised of linked 2′-deoxynucleotides positioned between a 5′ and a 3′ wing segment. In some embodiments, the modified sugar moiety is chosen from a 2′-O-methoxyethyl modified sugar moiety, a 2′-methoxy modified sugar moiety, a 2′-O-alkyl modified sugar moiety, or a bicyclic sugar moiety.

In some embodiments, the 5′-wing segment is 1 to 6 nucleotides in length. In some embodiments, the 3′-wing segment is 1 to 6 nucleotides in length. In some embodiments, the gap segment is 5 to 14 nucleotides in length. In some embodiments, the 5′-wing segment is 2 nucleotides in length. In some embodiments, the 3′-wing segment is 2 nucleotides in length. In some embodiments, the 5′-wing segment is 3 nucleotides in length. In some embodiments, the 3′-wing segment is 3 nucleotides in length. In some embodiments, the 5′-wing segment is 4 nucleotides in length. In some embodiments, the 3′-wing segment is 4 nucleotides in length. In some embodiments, the 5′-wing segment is 5 nucleotides in length. In some embodiments, the 3′-wing segment is 5 nucleotides in length. In some embodiments, the gap segment is 10 nucleotides in length.

In some embodiments, the antisense polynucleotide agent comprises: a gap segment consisting of linked deoxynucleotides; a 5′-wing segment consisting of linked nucleotides; a 3′-wing segment consisting of linked nucleotides; wherein the gap segment is positioned between the 5′-wing segment and the 3′-wing segment and wherein each nucleotide of each wing segment comprises a modified sugar.

In some embodiments, the gap segment is ten 2′-deoxynucleotides in length and each of the wing segments is five nucleotides in length. In some embodiments, the gap segment is ten 2′-deoxynucleotides in length and each of the wing segments is four nucleotides in length. In some embodiments, the gap segment is ten 2′-deoxynucleotides in length and each of the wing segments is three nucleotides in length. In some embodiments, the gap segment is ten 2′-deoxynucleotides in length and each of the wing segments is two nucleotides in length.

In some embodiments, the modified sugar moiety is selected from the group consisting of a 2′-O-methoxyethyl modified sugar moiety, a 2′-methoxy modified sugar moiety, a 2′-O-alkyl modified sugar moiety, and a bicyclic sugar moiety.

In some embodiments, the antisense polynucleotide agent further comprises a ligand. In some embodiments, the antisense polynucleotide agent is conjugated to the ligand at the 3′-terminus. In some embodiments, the ligand is an N-acetylgalactosamine (GalNAc) derivative.

In some embodiments, the ligand is

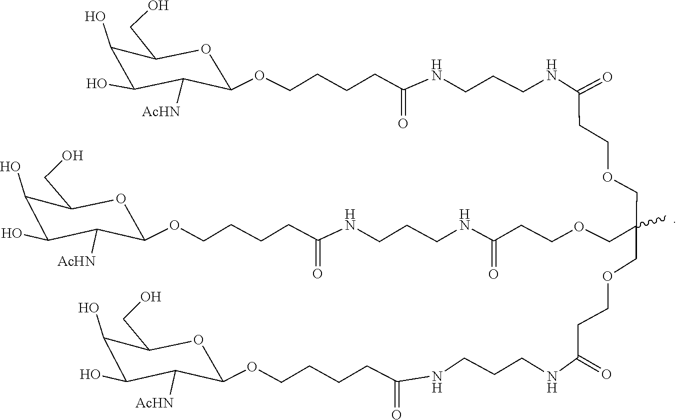 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In some embodiments, the dsRNA or antisense polynucleotide agent is administered to the subject using direct injection or infusion, intravenous, intraperitoneal, subcutaneous, intramuscular, inhalation, topical, intracranial, intracerebroventricular, epidural, intrathecal, intraarterial, intravitrial, intradermal, oral, or intracardiac delivery.

In some embodiments, the dsRNA or antisense polynucleotide agent is administered using intravenous delivery. In some embodiments, the dsRNA or antisense polynucleotide agent is administered to the subject in an unbuffered solution. In some embodiments, the unbuffered solution is saline or water. In some embodiments, the dsRNA or antisense polynucleotide agent is administered to the subject with a buffer solution. In some embodiments, the buffer solution comprises acetate, citrate, prolamine, carbonate, phosphate or any combination thereof. In some embodiments, the buffer solution is phosphate buffered saline (PBS).

In some embodiments, the dsRNA or antisense polynucleotide agent is administered to the subject in a pharmaceutical composition comprising a lipid formulation. In some embodiments, the lipid formulation is an LNP formulation. In some embodiments, the lipid formulation is an LNP11 formulation. In some embodiments, the dsRNA or antisense polynucleotide agent is targeted to a liver cell or a hepatocyte.

In some embodiments, the dsRNA or antisense polynucleotide agent is administered to the subject intravenously. In some embodiments, the dsRNA or antisense polynucleotide agent is administered to the subject subcutaneously.

In some embodiments, the dsRNA or antisense polynucleotide agent is administered to the subject intravenously in a pharmaceutical composition comprising a lipid formulation. In some embodiments, the dsRNA or antisense polynucleotide agent is conjugated to a ligand chosen from a carbohydrate ligand or a GalNAc ligand.

In some embodiments, the dsRNA or antisense polynucleotide agent is administered according to a dosing regimen. In some embodiments, the dosing regimen is weekly, biweekly, or monthly. In some embodiments, the dsRNA or antisense polynucleotide agent is administered to the subject once a week. In some embodiments, the dsRNA or antisense polynucleotide agent is administered to the subject twice a week. In some embodiments, the dsRNA or antisense polynucleotide agent is administered to the subject twice a month.

In some embodiments, the dsRNA or antisense polynucleotide agent is administered at a dose of about 0.01 mg/kg to about 100 mg/kg bodyweight of the subject. In some embodiments, the dsRNA or antisense polynucleotide agent is administered at a dose of about 0.05 mg/kg to about 50 mg/kg bodyweight of the subject. In some embodiments, the dsRNA or antisense polynucleotide agent is administered at a dose of about 0.01 mg/kg to about 5 mg/kg bodyweight of the subject. In some embodiments, the dsRNA or antisense polynucleotide agent is administered at a dose of about 0.1 mg/kg to about 0.5 mg/kg bodyweight of the subject. In some embodiments, the dsRNA or antisense polynucleotide agent is administered at a dose of about 0.5 mg/kg to about 10 mg/kg bodyweight of the subject. In some embodiments, the dsRNA or antisense polynucleotide agent is administered at a dose of about 1 mg/kg to about 10 mg/kg bodyweight of the subject.

In some embodiments, the disorder is amyloidosis. In some embodiments, the amyloidosis is a LECT2 amyloidosis (ALECT2). In some embodiments, the disorder is rheumatoid arthritis. In some embodiments, the disorder is an acute liver injury.

In some embodiments, the method further comprises administering a second therapy, to the subject. In some embodiments, the second therapy is a therapy that supports kidney function or a therapy that supports liver function. In some embodiments, the therapy that supports kidney function is selected from dialysis, a diuretic, an angiotensin converting enzyme (ACE) inhibitor, or an angiotensin receptor blocker (ARB). In some embodiments, the second therapy is removal of all or part of the organs affected by the amyloidosis.

All publications, patent applications, patents, and other references mentioned herein are incorporated by reference in their entirety.

The details of various embodiments of the invention are set forth in the description below. Other features, objects, and advantages of the invention will be apparent from the description and the drawings, and from the claims.

### BRIEF DESCRIPTION OF THE DRAWINGS

FIG. 1 depicts a human LECT2 mRNA transcript sequence (Ref. Seq. NM_002302.2 GI:59806344, record dated Apr. 17, 2013; SEQ ID NO: 1).

FIG. 2 is a graph showing the results of a circulating extracellular RNA detection (cERD) assay demonstrating that urine circulating extracellular LECT2 mRNA is higher in ALECT2 patients. Urine cERD LECT2 mRNA fold change is shown for four groups of subjects: healthy donors, chronic kidney disease (CKD) patients, amyloid light-chain (AL) amyloidosis patients, and LECT2 amyloidosis (ALECT2) patients. LECT2 Ct value is normalized to GAPDH Ct value. Fold change is calculated by assigning one of the healthy donors as 1 fold. Samples with undetectable LRCT2 mRNA level are arbitrarily assigned as 0.1 fold.

### DETAILED DESCRIPTION

The assays, compositions and methods described herein are based, in part, on the discovery that an LECT2 RNA can be detected by measuring levels of the LECT2 RNA in blood or urine, even when the LECT2 RNA is expressed in a different biological compartment (e.g., liver tissue). Thus, provided herein are methods and compositions for determining the activity or expression of a LECT2 gene, or the activity or efficacy of a nucleic acid agent (e.g., an iRNA (e.g., dsRNA) or antisense polynucleotide agent), by detecting LECT2 RNA in a bodily fluid sample (e.g., blood or urine) obtained from a subject. The subject can be a treatment naïve subject or a subject who has been administered a dsRNA or antisense polynucleotide agent that targets an LECT RNA in a tissue that is separate (e.g., distal) from the bodily fluid sample. The assays described herein are useful for monitoring the activity of agents other than dsRNAs or antisense polynucleotide agents. For example, the activity of other therapeutic agents, such as antagomirs, miRNA mimics and gene therapy agents, can be assayed by detecting target LECT2 RNA levels in a bodily fluid sample, such as a blood or urine sample, even when the therapeutic agent is active in a particular tissue distant from the site of sample collection.

A tissue that is separate from the bodily fluid sample can be distal to the sample. In one embodiment, the tissue contacts the bodily fluid sample, but is separated from the bodily fluid sample by a membrane, such as a lipid bilayer. For example, the tissue can be an organ, and the bodily fluid sample can be a fluid that contacts the tissue, such as in the case where the tissue is bathed in the bodily fluid sample. For example, the tissue can be the liver, and the bodily fluid sample used to detect LECT2 RNA levels in the liver can be a blood sample. The blood sample can be collected by methods known in the art, such as from a vein in the subject. In one embodiment, LECT2 RNA levels in a tissue can be monitored by assaying for LECT2 RNA levels in urine.

In one embodiment, a nucleic acid agent (e.g., an iRNA (e.g., a dsRNA) or antisense polynucleotide agent) that targets the liver can be monitored for interfering (e.g., silencing) activity by assaying an LECT2 RNA or a cleavage product thereof in the blood or urine. The methods and compositions described herein have the advantage of non-invasively determining the activity of a nucleic acid agent administered to a subject.

In certain embodiments, the nucleic acid agent is a dsRNA. The dsRNAs described herein include an RNA strand (the antisense strand) having a region which is 30 nucleotides or less in length, i.e., 15-30 nucleotides in length, generally 19-24 nucleotides in length, which region is substantially complementary to at least part of an mRNA transcript of a LECT2 gene (also referred to herein as an “LECT2-specific dsRNA”). The use of such a dsRNA allows the targeted degradation of mRNAs of genes that are implicated in disorders related to LECT2 expression, as described herein. Very low dosages of LECT2-specific dsRNAs can specifically and efficiently mediate RNAi, resulting in significant inhibition of expression of a LECT2 gene. DsRNAs targeting LECT2 can specifically and efficiently mediate RNAi, resulting in significant inhibition of expression of a LECT2 gene, which can be assessed, e.g., in cell based assays. In other embodiments, the nucleic acid agent is an antisense polynucleotide agent.

The antisense polynucleotide agents described herein bind nucleic acids encoding LECT2 via, e.g., Watson-Crick base pairing, and interfere with the normal function of the targeted nucleic acid. The antisense polynucleotide agents include a nucleotide sequence which is about 4 to about 50 nucleotides or less in length and which is about 80% complementary to at least part of an mRNA transcript of a LECT2 gene. The use of these antisense polynucleotide agents allows the targeted inhibition of RNA expression and/or activity of a LECT2 gene in mammals.

The present disclosure also provides methods and combination therapies for treating a subject having a disorder that would benefit from inhibiting or reducing the expression of a LECT2 gene, e.g., a LECT2-associated disease, such as amyloidosis, e.g. a LECT2 amyloidosis (ALECT2), using the iRNAs (e.g., dsRNAs) or antisense polynucleotide agents described herein. The present disclosure also provides methods for preventing at least one symptom, e.g., amyloid deposition, in a subject having a disorder that would benefit from inhibiting or reducing the expression of a LECT2 gene, e.g., a LECT2-associated disease, such as amyloidosis, e.g. a LECT2 amyloidosis (ALECT2), using the iRNAs (e.g., dsRNAs) or antisense polynucleotide agents described herein.

The following detailed description discloses how to make and use nucleic acid agents (e.g., iRNA (e.g., dsRNAs) or antisense polynucleotide agents) to inhibit the mRNA and/or protein expression of a LECT2 gene, as well as compositions, uses, and methods for treating subjects having diseases and disorders that would benefit from inhibition and/or reduction of the expression of this gene.

### I. Definitions

For convenience, the meaning of certain terms and phrases used in the specification, examples, and appended claims, are provided below. If there is an apparent discrepancy between the usage of a term in other parts of this specification and its definition provided in this section, the definition in this section shall prevail.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

The term “or” is used herein to mean, and is used interchangeably with, the term “and/or,” unless context clearly indicates otherwise.

As used herein, “LECT2” refers to leukocyte chemotactic factor 2 (also known as leukocyte cell-derived chemotaxin 2, chondromodulin-II, chm-II or chm2). See, e.g., Yamagoe et al. Genomics, 1998; 48(3):324-9. LECT2 was first identified as a novel neutrophil chemotactic protein and is identical with chondromodulin II, a growth stimulator for chondrocytes and osteoblasts. The human LECT2 gene was mapped to chromosome 5q31.1-q32. Ibid.

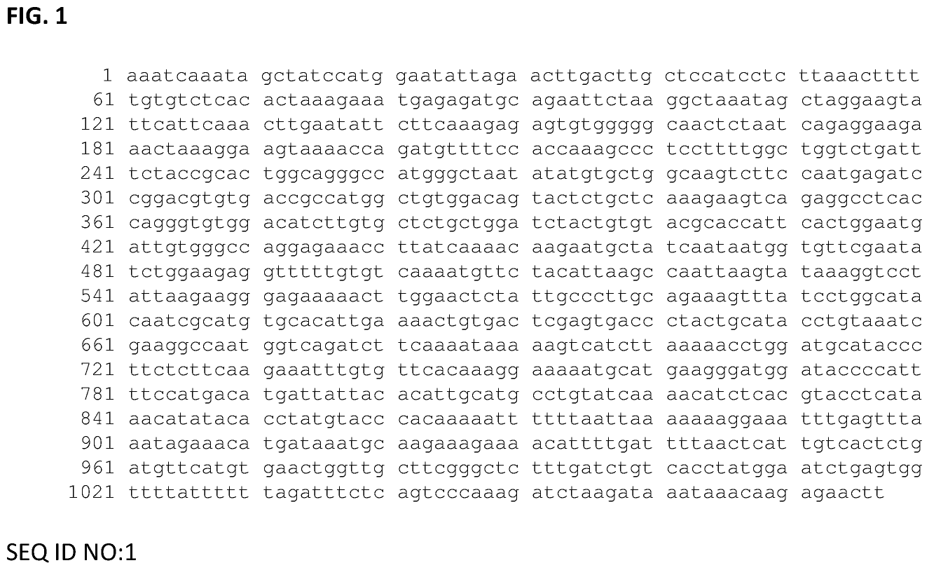

The sequence of a human LECT2 mRNA transcript can be found at NM_002302.2 (SEQ ID NO: 1; FIG. 1). The sequence of a mouse LECT2 mRNA can be found at NM_010702.1 and at NM_010702.2, and the sequence of a rat LECT2 mRNA can be found at NM_001108405.1.

The human LECT2 protein is a secreted, 16 kDa protein. The LECT2 protein is secreted by the liver. It has high sequence similarity to the chondromodulin repeat regions of the chicken myb-induced myeloid 1 protein (www.genecards.org/cgi-bin/carddisp.pl?gene=LECT2; accessed Aug. 29, 2013). Polymorphism in the LECT2 gene has been associated with rheumatoid arthritis. Ibid.

LECT2 is expressed in various tissues, including the brain and stomach as well as the liver. Koshimizu & Ohtomi (2010) Brain Res. 1311:1-11. In a study using indirect immunoperoxidase staining to investigate the expression of LECT2 in normal and diseased human organs and tissues other than liver, it was found that LECT2 was generally expressed in vascular, endothelial and smooth muscle cells, adipocytes, cerebral nerve cells, apical squamous epithelia, parathyroid cells, sweat and sebaceous glandular epithelia, Hassall bodies and some mononuclear cells in immunohematopoeietic tissue. This protein was generally negative, although occasionally positively stained in osteoblasts, chondrocytes, cardiac and skeletal muscle cells, smooth muscle cells of the gastrointestinal tract, and the epithelial cells of some tissues. Nagai et al. (1998) Pathol Int. 48(11):882-6.

The human LECT2 gene codes for 151 amino acids including an 18 amino acid signal peptide. The secreted protein has 133 residues. A G/A polymorphism at nucleotide 172 in exon 3 of the gene (codon change GTC to ATC) has been identified and accounts for the presence of either valine or isoleucine at position 58 of the unprocessed protein (or position 40 of the mature protein). The G allele has an overall frequency of 0.477 and a frequency range of 0.6-0.7 in individuals of European descent. See Benson et al. (2008) Kidney International, 74: 218-222; Murphy et al. (2010) Am J Kidney Dis, 56(6):1100-1107. Patients with LECT2 amyloidosis typically are homozygous for the G allele. Without wishing to be bound by theory, it has been suggested that replacement of the buried isoleucine (A allele) side chain with valine (G allele) could destabilize the protein and possibly account for the amyloidogenic propensity of this LECT2 variant. Murphy et al. (2010) Am J Kidney Dis, 56(6):1100-1107.

As used herein, a “LECT2 amyloidosis” or “ALECT2” includes an amyloidosis involving deposits of amyloid or amyloid fibrils that contain a LECT2 protein (e.g., any polymorphic variant of a LECT2 protein) or a portion of a LECT2 protein. The LECT2 protein can be a variant (e.g., a mutant) LECT2 protein. The amyloidosis can be systemic or local. In some embodiments, the LECT2 amyloidosis involves amyloid deposits in the kidney and/or liver.

“G,” “C,” “A,” “T” and “U” each generally stand for a nucleotide that contains guanine, cytosine, adenine, thymidine and uracil as a base, respectively. However, it will be understood that the term “ribonucleotide” or “nucleotide” can also refer to a modified nucleotide, as further detailed below, or a surrogate replacement moiety. The skilled person is well aware that guanine, cytosine, adenine, and uracil may be replaced by other moieties without substantially altering the base pairing properties of an oligonucleotide comprising a nucleotide bearing such replacement moiety. For example, without limitation, a nucleotide comprising inosine as its base may base pair with nucleotides containing adenine, cytosine, or uracil. Hence, nucleotides containing uracil, guanine, or adenine may be replaced in the nucleotide sequences of dsRNA featured in the invention by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the invention.

As used herein, the term “iRNA,” “RNAi”, “iRNA agent,” or “RNAi agent” refers to an agent that contains RNA as that term is defined herein, and which mediates the targeted cleavage of an RNA transcript, e.g., via an RNA-induced silencing complex (RISC) pathway. In one embodiment, an iRNA as described herein effects inhibition of LECT2 expression. Inhibition of ALECT2 expression may be assessed based on a reduction in the level of ALECT2 mRNA or a reduction in the level of the ALECT2 protein.

As used herein, “target sequence” refers to a contiguous portion of the nucleotide sequence of an mRNA molecule formed during the transcription of an ALECT2 gene, including mRNA that is a product of RNA processing of a primary transcription product.

For iRNAs, the target portion of the sequence will be at least long enough to serve as a substrate for iRNA-directed cleavage at or near that portion. For example, the target sequence will generally be from 9-36 nucleotides in length, e.g., 15-30 nucleotides in length, including all sub-ranges therebetween. As non-limiting examples, the target sequence can be from 15-30 nucleotides, 15-26 nucleotides, 15-23 nucleotides, 15-22 nucleotides, 15-21 nucleotides, 15-20 nucleotides, 15-19 nucleotides, 15-18 nucleotides, 15-17 nucleotides, 18-30 nucleotides, 18-26 nucleotides, 18-23 nucleotides, 18-22 nucleotides, 18-21 nucleotides, 18-20 nucleotides, 19-30 nucleotides, 19-26 nucleotides, 19-23 nucleotides, 19-22 nucleotides, 19-21 nucleotides, 19-20 nucleotides, 20-30 nucleotides, 20-26 nucleotides, 20-25 nucleotides, 20-24 nucleotides, 20-23 nucleotides, 20-22 nucleotides, 20-21 nucleotides, 21-30 nucleotides, 21-26 nucleotides, 21-25 nucleotides, 21-24 nucleotides, 21-23 nucleotides, or 21-22 nucleotides.

As used herein, the term “strand comprising a sequence” refers to an oligonucleotide comprising a chain of nucleotides that is described by the sequence referred to using the standard nucleotide nomenclature.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions may include: 400 mM NaCl, 40 mM PIPES pH 6.4, 1 mM EDTA, 50° C. or 70° C. for 12-16 hours followed by washing. Other conditions, such as physiologically relevant conditions as may be encountered inside an organism, can apply. The skilled person will be able to determine the set of conditions most appropriate for a test of complementarity of two sequences in accordance with the ultimate application of the hybridized nucleotides.

Complementary sequences within an iRNA, e.g., within a dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they may form one or more, but generally not more than 5, 4, 3 or 2 mismatched base pairs upon hybridization for a duplex up to 30 base pairs, while retaining the ability to hybridize under the conditions most relevant to their ultimate application, e.g., inhibition of gene expression via a RISC pathway. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, may yet be referred to as “fully complementary” for the purposes described herein.

“Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled. Such non-Watson-Crick base pairs include, but are not limited to, G:U Wobble or Hoogstein base pairing.

The terms “complementary,” “fully complementary” and “substantially complementary” herein may be used with respect to the base matching between the sense strand and the antisense strand of a dsRNA, or between the antisense strand of an iRNA agent and a target sequence, as will be understood from the context of their use.

As used herein, a polynucleotide that is “substantially complementary to at least part of” a messenger RNA (mRNA) refers to a polynucleotide that is substantially complementary to a contiguous portion of the mRNA of interest (e.g., an mRNA encoding an ALECT2 protein). For example, a polynucleotide is complementary to at least a part of a LECT2 mRNA if the sequence is substantially complementary to a non-interrupted portion of an mRNA encoding LECT2. As another example, a polynucleotide is complementary to at least a part of a LECT2 mRNA if the sequence is substantially complementary to a non-interrupted portion of an mRNA encoding LECT2.

The term “double-stranded RNA” or “dsRNA,” as used herein, refers to an iRNA that includes an RNA molecule or complex of molecules having a hybridized duplex region that comprises two anti-parallel and substantially complementary nucleic acid strands, which will be referred to as having “sense” and “antisense” orientations with respect to a target RNA. The duplex region can be of any length that permits specific degradation of a desired target RNA, e.g., through a RISC pathway, but will typically range from 9 to 36 base pairs in length, e.g., 15-30 base pairs in length. Considering a duplex between 9 and 36 base pairs, the duplex can be any length in this range, for example, 9, 10, 11, 12, 13, 14, 15, 16, 17, 18, 19, 20, 21, 22, 23, 24, 25, 26, 27, 28, 29, 30, 31, 32, 33, 34, 35, or 36 and any sub-range therein between, including, but not limited to 15-30 base pairs, 15-26 base pairs, 15-23 base pairs, 15-22 base pairs, 15-21 base pairs, 15-20 base pairs, 15-19 base pairs, 15-18 base pairs, 15-17 base pairs, 18-30 base pairs, 18-26 base pairs, 18-23 base pairs, 18-22 base pairs, 18-21 base pairs, 18-20 base pairs, 19-30 base pairs, 19-26 base pairs, 19-23 base pairs, 19-22 base pairs, 19-21 base pairs, 19-20 base pairs, 20-30 base pairs, 20-26 base pairs, 20-25 base pairs, 20-24 base pairs, 20-23 base pairs, 20-22 base pairs, 20-21 base pairs, 21-30 base pairs, 21-26 base pairs, 21-25 base pairs, 21-24 base pairs, 21-23 base pairs, or 21-22 base pairs. DsRNAs generated in the cell by processing with Dicer and similar enzymes are generally in the range of 19-22 base pairs in length. One strand of the duplex region of a dsDNA comprises a sequence that is substantially complementary to a region of a target RNA. The two strands forming the duplex structure can be from a single RNA molecule having at least one self-complementary region, or can be formed from two or more separate RNA molecules. Where the duplex region is formed from two strands of a single molecule, the molecule can have a duplex region separated by a single stranded chain of nucleotides (herein referred to as a “hairpin loop”) between the 3′-end of one strand and the 5′-end of the respective other strand forming the duplex structure. The hairpin loop can comprise at least one unpaired nucleotide; in some embodiments the hairpin loop can comprise at least 3, at least 4, at least 5, at least 6, at least 7, at least 8, at least 9, at least 10, at least 20, at least 23 or more unpaired nucleotides. Where the two substantially complementary strands of a dsRNA are comprised by separate RNA molecules, those molecules need not, but can be covalently connected. Where the two strands are connected covalently by means other than a hairpin loop, the connecting structure is referred to as a “linker.” The term “siRNA” is also used herein to refer to a dsRNA as described above.

In another embodiment, the iRNA agent may be a “single-stranded siRNA” that is introduced into a cell or organism to inhibit a target mRNA. Single-stranded RNAi agents bind to the RISC endonuclease Argonaute 2, which then cleaves the target mRNA. The single-stranded siRNAs are generally 15-30 nucleotides and are chemically modified. The design and testing of single-stranded siRNAs are described in U.S. Pat. No. 8,101,348 and in Lima et al., (2012) Cell 150: 883-894, the entire contents of each of which are hereby incorporated herein by reference. Any of the antisense nucleotide sequences described herein (e.g., sequences provided in Tables 2-5) may be used as a single-stranded siRNA as described herein or as chemically modified by the methods described in Lima et al., (2012) Cell 150: 883-894.

In another aspect, the iRNA agent is a “single-stranded antisense RNA molecule.” An single-stranded antisense RNA molecule is complementary to a sequence within the target mRNA. Single-stranded antisense RNA molecules can inhibit translation in a stoichiometric manner by base pairing to the mRNA and physically obstructing the translation machinery, see Dias, N. et al., (2002) Mol Cancer Ther 1:347-355. Alternatively, the single-stranded antisense molecules inhibit a target mRNA by hybridizing to the target and cleaving the target through an RNaseH cleavage event. The single-stranded antisense RNA molecule may be about 10 to about 30 nucleotides in length and have a sequence that is complementary to a target sequence. In one embodiment, the single-stranded antisense RNA molecule may comprise a sequence that is at least about 10, 11, 12, 13, 14, 15, 16, 17, 18, 19, 20, or more contiguous nucleotides complementary to any of the target sites described herein, e.g., sequences provided in any one of Tables 2-3, 5-6 and 9-10 of WO 2015/050990. In another embodiment, the single-stranded antisense RNA molecule may comprise a sequence that is at least about 10, 11, 12, 13, 14, 15, 16, 17, 18, 19, 20, or more contiguous nucleotides from any one of the antisense nucleotide sequences described herein, e.g., sequences provided in any one of Tables 2-3, 5-6 and 9-10 WO2015/050990.

The terms “antisense polynucleotide agent” “antisense compound”, and “agent” as used interchangeably herein, refer to an agent comprising a single-stranded oligonucleotide that contains RNA as that term is defined herein, and which targets nucleic acid molecules encoding LECT2 (e.g., mRNA encoding LECT2 as provided in, for example, any one of SEQ ID NOs:1-4 of International Application Publication No. WO2016/164746). The antisense polynucleotide agents specifically bind to the target nucleic acid molecules via hydrogen bonding (e.g., Watson-Crick, Hoogsteen or reversed Hoogsteen hydrogen bonding) and interfere with the normal function of the targeted nucleic acid (e.g., by an antisense mechanism of action). This interference with or modulation of the function of a target nucleic acid by the polynucleotide agents of the present disclosure is referred to as “antisense inhibition.”

The functions of the target nucleic acid molecule to be interfered with may include functions such as, for example, translocation of the RNA to the site of protein translation, translation of protein from the RNA, splicing of the RNA to yield one or more mRNA species, and catalytic activity which may be engaged in or facilitated by the RNA.

In some embodiments, antisense inhibition refers to “inhibiting the expression” of target nucleic acid levels and/or target protein levels in a cell, e.g., a cell within a subject, such as a mammalian subject, in the presence of the antisense polynucleotide agent complementary to a target nucleic acid as compared to target nucleic acid levels and/or target protein levels in the absence of the antisense polynucleotide agent. For example, the antisense polynucleotide agents of the invention can inhibit translation in a stoichiometric manner by base pairing to the mRNA and physically obstructing the translation machinery, see Dias, N. et al., (2002) Mol Cancer Ther 1:347-355.

As used herein, “target nucleic acid” refers to a nucleic acid molecule to which an iRNA (e.g., one strand of an iRNA) or an antisense polynucleotide agent specifically hybridizes.

As used herein, the term “specifically hybridizes” refers to an iRNA (e.g., one strand of an iRNA) or an antisense polynucleotide agent having a sufficient degree of complementarity between the antisense polynucleotide agent and a target nucleic acid to induce a desired effect, while exhibiting minimal or no effects on non-target nucleic acids under conditions in which specific binding is desired, e.g., under physiological conditions in the case of in vivo assays and therapeutic treatments.

For an antisense polynucleotide agent, a target sequence may be from about 4-50 nucleotides in length, e.g., 8-45, 10-45, 10-40, 10-35, 10-30, 10-20, 11-45, 11-40, 11-35, 11-30, 11-20, 12-45, 12-40, 12-35, 12-30, 12-25, 12-20, 13-45, 13-40, 13-35, 13-30, 13-25, 13-20, 14-45, 14-40, 14-35, 14-30, 14-25, 14-20, 15-45, 15-40, 15-35, 15-30, 15-25, 15-20, 16-45, 16-40, 16-35, 16-30, 16-25, 16-20, 17-45, 17-40, 17-35, 17-30, 17-25, 17-20, 18-45, 18-40, 18-35, 18-30, 18-25, 18-20, 19-45, 19-40, 19-35, 19-30, 19-25, 19-20, e.g., 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 contiguous nucleotides of the nucleotide sequence of an mRNA molecule formed during the transcription of a LECT2 gene. Ranges and lengths intermediate to the above recited ranges and lengths are also contemplated to be part of the disclosure.

The skilled artisan will recognize that the term “RNA molecule” or “ribonucleic acid molecule” encompasses not only RNA molecules as expressed or found in nature, but also analogs and derivatives of RNA comprising one or more ribonucleotide/ribonucleoside analogs or derivatives as described herein or as known in the art. Strictly speaking, a “ribonucleoside” includes a nucleoside base and a ribose sugar, and a “ribonucleotide” is a ribonucleoside with one, two or three phosphate moieties. However, the terms “ribonucleoside” and “ribonucleotide” can be considered to be equivalent as used herein. The RNA can be modified in the nucleobase structure, in the ribose structure, or in the ribose-phosphate backbone structure, e.g., as described herein below. However, the molecules comprising ribonucleoside analogs or derivatives must retain the ability to form a duplex. As non-limiting examples, an RNA molecule can also include at least one modified ribonucleoside including but not limited to a 2′-O-methyl modified nucleoside, a nucleoside comprising a 5′ phosphorothioate group, a terminal nucleoside linked to a cholesteryl derivative or dodecanoic acid bisdecylamide group, a locked nucleoside, an abasic nucleoside, an acyclic nucleoside, a 2′-deoxy-2′-fluoro modified nucleoside, a 2′-amino-modified nucleoside, 2′-alkyl-modified nucleoside, morpholino nucleoside, a phosphoramidate or a non-natural base comprising nucleoside, or any combination thereof. Alternatively, an RNA molecule can comprise at least two modified ribonucleosides, at least 3, at least 4, at least 5, at least 6, at least 7, at least 8, at least 9, at least 10, at least 15, at least 20 or more, up to the entire length of the dsRNA molecule. The modifications need not be the same for each of such a plurality of modified ribonucleosides in an RNA molecule. In one embodiment, modified RNAs contemplated for use in methods and compositions described herein are peptide nucleic acids (PNAs) that have the ability to form the required duplex structure and that permit or mediate the specific degradation of a target RNA, e.g., via a RISC pathway.

In one embodiment, a modified ribonucleoside includes a deoxyribonucleoside. In such an instance, an iRNA agent can comprise one or more deoxynucleosides, including, for example, a deoxynucleoside overhang(s), or one or more deoxynucleosides within the double stranded portion of a dsRNA. In certain embodiments, the RNA molecule comprises a percentage of deoxyribonucleosides of at least 5, 10, 15, 20, 25, 30, 35, 40, 45, 50, 55, 60, 65, 70, 75, 80, 85, 90, 95% or higher (but not 100%) deoxyribonucleosides, e.g., in one or both strands. In other embodiments, the term “iRNA” does not encompass a double stranded DNA molecule (e.g., a naturally-occurring double stranded DNA molecule or a 100% deoxynucleoside-containing DNA molecule).

In one embodiment, an iRNA includes a single stranded RNA that interacts with a target RNA sequence to direct the cleavage of the target RNA. Without wishing to be bound by theory, long double stranded RNA introduced into cells is broken down into siRNA by a Type III endonuclease known as Dicer (Sharp et al., Genes Dev. 2001, 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleaves the target to induce silencing (Elbashir, et al., (2001) Genes Dev. 15:188). Thus, in one aspect the invention relates to a single stranded RNA that promotes the formation of a RISC complex to effect silencing of the target gene.

As used herein, the term “nucleotide overhang” refers to at least one unpaired nucleotide that protrudes from the duplex structure of an iRNA, e.g., a dsRNA. For example, when a 3′-end of one strand of a dsRNA extends beyond the 5′-end of the other strand, or vice versa, there is a nucleotide overhang. A dsRNA can comprise an overhang of at least one nucleotide; alternatively the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) may be on the sense strand, the antisense strand or any combination thereof. Furthermore, the nucleotide(s) of an overhang can be present on the 5′ end, 3′ end or both ends of either an antisense or sense strand of a dsRNA.

In one embodiment, the antisense strand of a dsRNA has a 1-10 nucleotide overhang at the 3′ end and/or the 5′ end. In one embodiment, the sense strand of a dsRNA has a 1-10 nucleotide overhang at the 3′ end and/or the 5′ end. In another embodiment, one or more of the nucleotides in the overhang is replaced with a nucleoside thiophosphate.

The terms “blunt” or “blunt ended” as used herein in reference to a dsRNA mean that there are no unpaired nucleotides or nucleotide analogs at a given terminal end of a dsRNA, i.e., no nucleotide overhang. One or both ends of a dsRNA can be blunt. Where both ends of a dsRNA are blunt, the dsRNA is said to be blunt ended. To be clear, a “blunt ended” dsRNA is a dsRNA that is blunt at both ends, i.e., no nucleotide overhang at either end of the molecule. Most often such a molecule will be double-stranded over its entire length.

The term “antisense strand” or “guide strand” refers to the strand of an iRNA, e.g., a dsRNA, which includes a region that is substantially complementary to a target sequence. As used herein, the term “region of complementarity” refers to the region on the antisense strand that is substantially complementary to a sequence, for example a target sequence, as defined herein. Where the region of complementarity is not fully complementary to the target sequence, the mismatches may be in the internal or terminal regions of the molecule. Generally, the most tolerated mismatches are in the terminal regions, e.g., within 5, 4, 3, or 2 nucleotides of the 5′ and/or 3′ terminus.

The term “sense strand” or “passenger strand” as used herein, refers to the strand of an iRNA that includes a region that is substantially complementary to a region of the antisense strand as that term is defined herein.

As used herein, the term “SNALP” refers to a stable nucleic acid-lipid particle. A SNALP represents a vesicle of lipids coating a reduced aqueous interior comprising a nucleic acid such as an iRNA or a plasmid from which an iRNA is transcribed. SNALPs are described, e.g., in U.S. Patent Application Publication Nos. 2006/0240093, 2007/0135372, and in International Application Publication No. WO 2009/082817. These applications are incorporated herein by reference in their entirety.

“Introducing into a cell,” when referring to an iRNA, means facilitating or effecting uptake or absorption into the cell, as is understood by those skilled in the art. Absorption or uptake of an iRNA can occur through unaided diffusive or active cellular processes, or by auxiliary agents or devices. The meaning of this term is not limited to cells in vitro; an iRNA may also be “introduced into a cell,” wherein the cell is part of a living organism. In such an instance, introduction into the cell will include the delivery to the organism. For example, for in vivo delivery, iRNA can be injected into a tissue site or administered systemically. In vivo delivery can also be by a β-glucan delivery system, such as those described in U.S. Pat. Nos. 5,032,401 and 5,607,677, and U.S. Application Publication No. 2005/0281781, which are hereby incorporated by reference in their entirety. In vitro introduction into a cell includes methods known in the art such as electroporation and lipofection. Further approaches are described herein below or known in the art.

As used herein, the term “modulate the expression of,” refers to at an least partial “inhibition” or partial “activation” of a LECT2 gene expression in a cell treated with an iRNA composition as described herein compared to the expression of LECT2 in a control cell. A control cell includes an untreated cell, or a cell treated with a non-targeting control iRNA. The terms “activate,” “enhance,” “up-regulate the expression of,” “increase the expression of,” and the like, in so far as they refer to a LECT2 gene, herein refer to the at least partial activation of the expression of a LECT2 gene, as manifested by an increase in the amount of LECT2 mRNA, which may be isolated from or detected in a first cell or group of cells in which a LECT2 gene is transcribed and which has or have been treated such that the expression of a LECT2 gene is increased, as compared to a second cell or group of cells substantially identical to the first cell or group of cells but which has or have not been so treated (control cells).

In one embodiment, expression of a LECT2 gene is activated by at least about 10%, 15%, 20%, 25%, 30%, 35%, 40%, 45%, or 50% by administration of an iRNA as described herein. In some embodiments, a LECT2 gene is activated by at least about 60%, 70%, or 80% by administration of an iRNA featured in the invention. In some embodiments, expression of a LECT2 gene is activated by at least about 85%, 90%, or 95% or more by administration of an iRNA as described herein. In some embodiments, the LECT2 gene expression is increased by at least 1-fold, at least 2-fold, at least 5-fold, at least 10-fold, at least 50-fold, at least 100-fold, at least 500-fold, at least 1000 fold or more in cells treated with an iRNA as described herein compared to the expression in an untreated cell. Activation of expression by small dsRNAs is described, for example, in Li et al., 2006 Proc. Natl. Acad. Sci. U.S.A. 103:17337-42, and in US2007/0111963 and US2005/226848, each of which is incorporated herein by reference.

The terms “silence,” “inhibit expression of,” “down-regulate expression of,” “suppress expression of,” and the like, in so far as they refer to a LECT2 gene, herein refer to the at least partial suppression of the expression of a LECT2 gene, as assessed, e.g., based on LECT2 mRNA expression, LECT2 protein expression, or another parameter functionally linked to LECT2 gene expression. For example, inhibition of LECT2 expression may be manifested by a reduction of the amount of LECT2 mRNA which may be isolated from or detected in a first cell or group of cells in which a LECT2 gene is transcribed and which has or have been treated such that the expression of a LECT2 gene is inhibited, as compared to a control. The control may be a second cell or group of cells substantially identical to the first cell or group of cells, except that the second cell or group of cells have not been so treated (control cells). The degree of inhibition is usually expressed as a percentage of a control level, e.g.,

$$\frac{( mRNA in control cells ) - ( mRNA in treated cells )}{( mRNA in control cells )} \cdot 100 \% .$$

Alternatively, the degree of inhibition may be given in terms of a reduction of a parameter that is functionally linked to LECT2 gene expression, e.g., the amount of protein encoded by a LECT2 gene. The reduction of a parameter functionally linked to LECT2 gene expression may similarly be expressed as a percentage of a control level. In principle, LECT2 gene silencing may be determined in any cell expressing LECT2, either constitutively or by genomic engineering, and by any appropriate assay.

As used herein, the term “miRNA,” or “microRNA” refers to a short RNA sequence (e.g., about 22 nucleotides) produced by a eukaryotic cell that acts as a post-transcriptional regulator by binding to complementary sequences on target mRNAs. MicroRNAs typically induce translational repression and gene silencing.

For example, in certain instances, expression of a LECT2 gene is suppressed by at least about 10%, 15%, 20%, 25%, 30%, 35%, 40%, 45%, or 50% by administration of an iRNA disclosed herein. In some embodiments, a LECT2 gene is suppressed by at least about 60%, 65%, 70%, 75%, or 80% by administration of an iRNA disclosed herein. In some embodiments, a LECT2 gene is suppressed by at least about 85%, 90%, 95%, 98%, 99%, or more by administration of an iRNA as described herein.

In the context of the present disclosure, the terms “treat,” “treatment,” and the like mean to prevent, relieve or alleviate at least one symptom associated with a disorder related to LECT2 expression, or to slow or reverse the progression or anticipated progression of such a disorder. For example, the methods featured herein, when employed to treat a LECT2 amyloidosis, may serve to inhibit amyloid deposition, to reduce or prevent one or more symptoms of the amyloidosis, or to reduce the risk or severity of associated conditions (e.g., renal insufficiency or nephrotic syndrome or hepatitis). Thus, unless the context clearly indicates otherwise, the terms “treat,” “treatment,” and the like are intended to encompass prophylaxis, e.g., prevention of disorders and/or symptoms of disorders related to LECT2 expression.

By “lower” in the context of a disease marker or symptom is meant any decrease, e.g., a statistically or clinically significant decrease in such level. The decrease can be, for example, at least 10%, at least 20%, at least 30%, at least 40%, at least 50%, at least 60%, at least 70%, at least 80%, or at least 90%. The decrease can be down to a level accepted as within the range of normal for an individual without such disorder.

As used herein, the phrases “therapeutically effective amount” and “prophylactically effective amount” and the like refer to an amount that provides a therapeutic benefit in the treatment, prevention, or management of any disorder or pathological process related to LECT2 expression. The specific amount that is therapeutically effective may vary depending on factors known in the art, such as, for example, the type of disorder or pathological process, the patient's history and age, the stage of the disorder or pathological process, and the administration of other therapies.

As used herein, a “pharmaceutical composition” comprises a pharmacologically effective amount of an iRNA and a pharmaceutically acceptable carrier. As used herein, “pharmacologically effective amount,” “therapeutically effective amount” or simply “effective amount” refers to that amount of an iRNA effective to produce the intended pharmacological, therapeutic or preventive result. For example, in a method of treating a disorder related to LECT2 expression (e.g., a LECT2 amyloidosis), an effective amount includes an amount effective to reduce one or more symptoms associated with the LECT2 amyloidosis, an amount effective to inhibit amyloid deposition (e.g., LECT2 amyloid deposition), or an amount effective to reduce the risk of developing conditions associated with LECT2 amyloidosis. For example, if a given clinical treatment is considered effective when there is at least a 10% reduction in a measurable parameter associated with a disease or disorder, a therapeutically effective amount of a drug for the treatment of that disease or disorder is the amount necessary to obtain at least a 10% reduction in that parameter. For example, a therapeutically effective amount of an iRNA targeting LECT2 can reduce a level of LECT2 mRNA or a level of LECT2 protein by any measurable amount, e.g., by at least 10%, 20%, 30%, 40% or 50%.

The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract. Agents included in drug formulations are described further herein below.

As used herein, the terms “distal from the biological sample” and “separate from the biological sample” refer to a site distinct from the biological sample obtained from a subject for analysis; that is, the biological sample obtained is not the intended site at which the iRNA modulates expression of a LECT2 gene. In some embodiments, the biological sample is a bodily fluid sample. In one embodiment, the biological sample is not obtained from the same tissue in which the LECT2 gene is targeted for expression. For example, the biological sample obtained from the subject is blood (e.g., serum or plasma) or urine and a LECT2 gene can be expressed in liver tissue. In one embodiment, the tissue contacts the biological sample, but is separated from the biological sample by a membrane, such as a lipid bilayer. For example, the tissue can be an organ, and the biological sample can be a fluid that contacts the tissue, such as in the case where the tissue is bathed in the biological sample. For example, the tissue can be the liver, and the biological sample used to monitor RNA levels in the liver can be a blood sample (e.g., a serum or plasma sample) or a urine sample. The biological sample can be collected by methods known in the art. For example, a blood sample can be collected from a vein in the subject.

“Acquire” or “acquiring” as the terms are used herein, refer to obtaining possession of a physical entity, or a value, e.g., a numerical value, by “directly acquiring” or “indirectly acquiring” the physical entity or value. “Directly acquiring” means performing a process (e.g., performing a synthetic or analytical method) to obtain the physical entity or value. “Indirectly acquiring” refers to receiving the physical entity or value from another party or source (e.g., a third party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

“Acquiring a sample” as the term is used herein, refers to obtaining possession of a sample, e.g., a tissue sample or nucleic acid sample, by “directly acquiring” or “indirectly acquiring” the sample. “Directly acquiring a sample” means performing a process (e.g., performing a physical method such as a surgery or extraction) to obtain the sample. “Indirectly acquiring a sample” refers to receiving the sample from another party or source (e.g., a third party laboratory that directly acquired the sample). Directly acquiring a sample includes performing a process that includes a physical change in a physical substance, e.g., a starting material, such as a tissue, e.g., a tissue in a human patient or a tissue that has was previously isolated from a patient. Exemplary changes include making a physical entity from a starting material, dissecting or scraping a tissue; separating or purifying a substance (e.g., a sample tissue or a nucleic acid sample); combining two or more separate entities into a mixture; performing a chemical reaction that includes breaking or forming a covalent or non-covalent bond. Directly acquiring a sample includes performing a process that includes a physical change in a sample or another substance, e.g., as described above.

The term “about” when referring to a number or a numerical range means that the number or numerical range referred to is an approximation within experimental variability (or within statistical experimental error), and thus the number or numerical range may vary from, for example, between 1% and 15% of the stated number or numerical range.

Nucleic acid sequences provided herein are represented using standard nomenclature. See the abbreviations of Table 1.

TABLE 1
Abbreviations of nucleotide monomers used in nucleic acid sequence representation. It will be understood that these monomers, when present in an oligo-
nucleotide, are mutually linked by 5′-3′-phosphodiester bonds.

| Abbreviation | Nucleotide(s) |
|---|---|
| A | Adenosine-3′-phosphate |
| Ab | beta-L-adenosine-3′-phosphate |
| Abs | beta-L-adenosine-3′-phosphorothioate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cb | beta-L-cytidine-3′-phosphate |
| Cbs | beta-L-cytidine-3′-phosphorothioate |
| Cf | 2′-fluorocytidine-3′-phosphate |
| Cfs | 2′-fluorocytidine-3′-phosphorothioate |
| (Chd) | 2′-O-hexadecyl-cytidine-3′-phosphate |
| (Chds) | 2′-O-hexadecyl-cytidine-3′-phosphorothioate |
| Cs | cytidine-3′-phosphorothioate |
| G | guanosine-3′-phosphate |
| Gb | beta-L-guanosine-3′-phosphate |
| Gbs | beta-L-guanosine-3′-phosphorothioate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tb | beta-L-thymidine-3′-phosphate |
| Tbs | beta-L-thymidine-3′-phosphorothioate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | Uridine-3′-phosphate |
| Ub | beta-L-uridine-3′-phosphate |
| Ubs | beta-L-uridine-3′-phosphorothioate |
| Uf | 2′-fluorouridine-3′-phosphate |
| Ufs | 2′-fluorouridine-3′-phosphorothioate |
| (Uhd) | 2′-O-hexadecyl-uridine-3′-phosphate |
| (Uhds) | 2′-O-hexadecyl-uridine-3′-phosphorothioate |
| Us | uridine-3′-phosphorothioate |
| N | any nucleotide (G, A, C, T or U) |
| a | 2′-O-methyladenosine-3′-phosphate |
| as | 2′-O-methyladenosine-3′-phosphorothioate |
| c | 2′-O-methylcytidine-3′-phosphate |
| cs | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| dA | 2′-deoxyadenosine-3′-phosphate |
| dAs | 2′-deoxyadenosine-3′-phosphorothioate |
| dC | 2′-deoxycytidine-3′-phosphate |
| dCs | 2′-deoxycytidine-3′-phosphorothioate |
| dG | 2′-deoxyguanosine-3′-phosphate |
| dGs | 2′-deoxyguanosine-3′-phosphorothioate |
| dT | 2′-deoxythymidine |
| dTs | 2′-deoxythymidine-3′-phosphorothioate |
| dU | 2′-deoxyuridine |
| s | phosphorothioate linkage |
| L96¹ | N-[tris(GalNAc-alkyl)-amidodecanoyl)]-4-hydroxyprolinol; Hyp-(GalNAc-alkyl)3 |
| (Aeo) | 2′-O-methoxyethyladenosine-3′-phosphate |
| (Aeos) | 2′-O-methoxyethyladenosine-3′-phosphorothioate |
| (Geo) | 2′-O-methoxyethylguanosine-3′-phosphate |
| (Geos) | 2′-O-methoxyethylguanosine-3′-phosphorothioate |
| (Teo) | 2′-O-methoxyethyl-5-methyluridine-3′-phosphate |
| (Teos) | 2′-O-methoxyethyl-5-methyluridine-3′-phosphorothioate |
| (m5Ceo) | 2′-O-methoxyethyl-5-methylcytidine-3′-phosphate |
| (m5Ceos) | 2′-O-methoxyethyl-5-methylcytidine-3′-phosphorothioate |

¹The chemical structure of L96 is as follows:

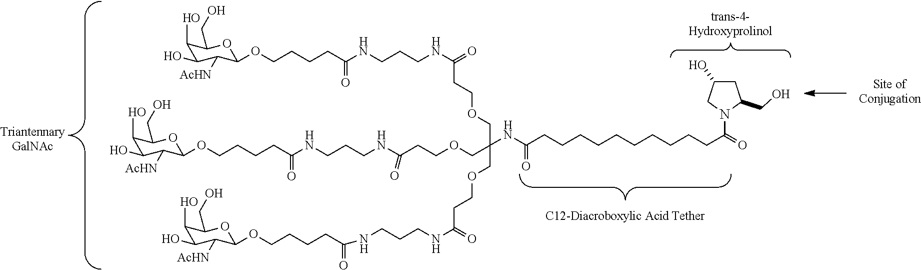

### II. Assays

Provided herein are assays for determining the activity or expression of a LECT2 gene, or the activity of a nucleic acid agent (e.g., an iRNA (e.g., dsRNA) or antisense polynucleotide agent), in a subject, by detecting expression of an LECT2 RNA or a cleavage product thereof in a biological sample (e.g., a bodily fluid sample) obtained from the subject. The assays described herein can be used to evaluating or identifying a subject having a LECT2-associated disorder, or at risk of having a LECT2-associated disorder. The assays described herein can also be used to assess or monitor a therapy for a LECT2-associated disorder, e.g., a therapy comprising a nucleic acid agent (e.g., an iRNA (e.g., dsRNA) or antisense polynucleotide agent). The assays described herein permit detection of LECT2 RNA levels in a biological sample (e.g., a bodily fluid sample) that is separate (e.g., distal) from the tissue where the LECT2 RNA is expressed. Such assays have the advantage of obtaining a biological sample in a non-invasive manner to determine whether a subject has a LECT2-associated disorder or whether a subject having a LECT2-associated disorder has responded to a therapy.

### Biological Samples

Various biological samples can be used to measure mRNA expression levels of a LECT2 target gene. Typically, the biological sample is a biological fluid such as blood (e.g., serum or plasma), urine, cerebrospinal fluid (CSF), amniotic fluid, saliva, breast milk, bronchoalveolar lavage fluid, synovial fluid, sputum, spinal fluid, pleural fluid, nipple aspirates, lymph fluid, fluid of the respiratory, intestinal, and genitourinary tracts, tear fluid, fluid from the lymphatic system, semen, intra-organ system fluid, malignant ascites, tumor cyst fluid, or a combination thereof.

In one embodiment, the biological sample comprises circulating extracellular RNA. In one embodiment, the biological sample comprises exosomal RNA. In one embodiment, the biological sample comprises circulating extracellular RNA and exosomal RNA.

In one embodiment, the biological sample is separate (e.g., distal) from the tissue where the LECT2 gene is expressed. In another embodiment, the biological sample is separate (e.g., distal) from the tissue where the expression of the LECT2 gene is inhibited (e.g., silenced or interfered). In another embodiment, the biological sample is separate (e.g., distal) from the tissue where a symptom of a LECT2-associated disorder (e.g., a LECT2-associated disorder described herein) is manifested.

In one embodiment, the biological sample is a bodily fluid sample. In one embodiment, the biological sample is blood, e.g., serum or plasma. In another embodiment, the biological sample is urine.

In some embodiments, a biological sample that can be obtained using methods having minimal invasiveness (e.g., blood (e.g., serum or plasma) or urine) is desired. The methods and assays provided herein provide an advantage of permitting diagnosis of a LECT2-associated disorder (e.g., ALECT2) or detection of successful dsRNA- or antisense-mediated silencing using minimally invasive techniques and avoiding costly and/or risky tissue biopsies (e.g., liver or kidney biopsy).

A biological sample can be obtained from a subject using methods known to those of skill in the art, e.g., a physician or the like. For example, the sample can be obtained using a syringe or a swab.

A biological sample can be obtained, or provided, directly or indirectly. Directly obtaining, or providing, means performing a process to obtain the sample. Indirectly obtaining, or providing, refers to receiving the sample from another party or source, such as from a third party laboratory that directly obtained the sample.

The biological sample can be stored prior to detecting target RNA levels. Typically, the lower the temperature the longer the sample can stably be stored. In one embodiment, the temperature is between −5° C. and −80° C. In other embodiments, the storage temperature is between −15° C. and −20° C. In other embodiments, the storage temperature is −20° C. or −80° C. prior to isolation of RNA from the sample. In another embodiment, the sample may be stored at 4° C. for, e.g., 2 hours to 168 hours prior to RNA isolation from the sample.

The biological sample can be subjected to filtration, such as through a 0.2 μM to 0.5 μM filter, e.g., through a 0.4 μM filter, prior to RNA isolation. Further, or in the alternative, the biological sample can be subjected to centrifugation, such as high speed centrifugation, prior to RNA isolation from the sample. For example, the sample can be centrifuged at 1000 g to 10,000 g, e.g., 2000 g to 5000 g. In some embodiments, the sample is centrifuged (e.g., is further centrifuged) at 100,000 g to 300,000 g, e.g., 150,000 g to 250,000 g, e.g., 200,000 g. In some embodiments, LiCl is added to the sample before one or more of the centrifugation steps. LiCl can be added at a concentration of, for example, 0.5 M, 1 M, 1.5 M, 2 M, or more.

### Exosomes

In some embodiments, the biological sample (e.g., bodily fluid sample) contains exosomes. The term “exosome” as used herein is a nanometer-sized (30 nm to 150 nm, e.g., 40 nm to 100 nm) vesicle that originates as an internal vesicle of a multivesicular body (MVB), present in endocytic and secretory pathways. Exosomes are formed by an invagination process or inward budding which causes a membrane-enclosed compartment in which the lumen is topologically equivalent of cytoplasm. “Microvesicles” are larger vesicles (e.g., 100 nm to 1000 nm) produced by a budding process from lipid raft regions of the plasma membrane.

RNAs, including mRNA and miRNA are internalized within exosomes. Exosomes are released in a regulated fashion into the extracellular environment by different cell types under both normal and pathological conditions, and contain RNA, protein and intracellular organelles from the cells where they originated. Cell types that express exosomes include neural cells, liver cells, and cells of most other organs. Their protein, RNA (e.g., mRNA and miRNA) and lipid composition are a consequence of sorting events at the level of the microvesicle body. Exosomes include common as well as cell-type specific proteins and RNA. Exosomes are present in many types of bodily fluids, including blood, plasma, serum, cerebrospinal fluid, urine, saliva, amniotic fluid, breast milk, bronchoalveolar lavage and synovial fluids, and malignant ascites.

In some embodiments, a fraction of the LECT2 RNA or cleavage product thereof in the biological sample is present in exosomes. For example, typically at least 0.000001% of the LECT2 RNA or cleavage product thereof in the biological sample is present in exosomes; For example at least 0.00001%, at least 0.001%, at least 0.01%, at least 1%, at least 2%, at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 95%, at least 99%, or even 100% (i.e., the entire fraction) of the LECT2 RNA or cleavage product may be present in exosomes. For example, circulating extracellular RNA or exosomal RNA for liver markers is approximately 10⁵ to 10⁶ lower than the liver cellular RNA (e.g., 0.00001% to 0.000001% of the RNA is in the exosomes).

In one embodiment, a biological sample (e.g., bodily fluid sample) is enriched for exosomes. A biological sample can be enriched for exosomes by, for example, centrifugation and/or chromatography, such as size-exclusion chromatography. A biological sample can be enriched for exosomes, but may not include exosomes that have been “purified.” Exosomes are purified through the use of exosome-binding molecules, such as antibodies, leptins or other protein ligands.

In one embodiment, exosomes are not purified prior to detection of the LECT2 RNA (e.g., mRNA) or cleavage product thereof (e.g., RISC cleavage product). Exosomes that have not been purified from a biological sample have not been selected for using antibodies or leptins or other protein ligands. For example, the exosomes are not purified by selecting for proteins that are enriched on exosomes, such as by using antibodies or leptins or other protein ligands. Proteins enriched on exosomes include, e.g., chaperones, tetraspanins, adhesion molecules, rab proteins, cytoskeletal proteins and metabolic enzymes. Exemplary proteins associated with exosomes include, e.g., CD63, CD9, beta1-integrin, CD81, ICAM-1, Mfg-E8, transferrin receptor, sialic acid, mucins, Tsg101 (Tumor susceptibility gene 101), Aip1/Alix, annexin II, EF1a (Transcription Elongation factor 1a), CD82, ceramide, and sphingomyelin (e.g., Conde-Vancells et al., J. Proteome Res. 7:5157-5166, 2008).

“Enriched” as used herein refers to a sample that is selected, processed, or manipulated to contain a greater percentage of a particular component (e.g., a biological sample enriched for exosomes, or enriched for more liver-specific exosomes) than the sample contained prior to the manipulation. For example, an enriched sample comprises at least 10% of the desired component (e.g., exosomes); in other embodiments, the enriched sample comprises at least 20%, at least 30%, at least 40%, at least 50%, at least 60%, at least 70%, at least 80%, at least 90%, at least 95%, or at least 99% of the desired component. Thus, an exosome-enriched sample refers to a sample that comprises at least 10% exosomes as determined by, e.g., measuring the level of an exosome cell surface antigen such as those described in e.g., U.S. Pat. No. 7,198,923. In other embodiments, an exosome-enriched sample comprises e.g., at least 20%, at least 30%, at least 40%, at least 50%, at least 60%, at least 70%, at least 80%, at least 90%, at least 95%, or at least 99% exosomes.

As used herein, “purified” means substantially separated from other components of the biological sample, e.g., cells and cell-free proteins and nucleic acids. Components are purified, for example, using antibodies or leptins or other protein ligands to separate the purified component from other components in the biological sample.

In some embodiments, exosomes derived from a tissue where gene silencing is targeted are not selected for prior to detecting the RNA. As used herein, the term “selected for” refers to purification of vesicles by a marker, such as a cell surface marker. For example, an antibody, such as a monoclonal or polyclonal antibody, or a peptide ligand, is not used to select for a tissue specific marker on the surface of an exosome, such as to purify the exosome from a specific tissue, such as from the liver. Exosomes are not purified by selecting for proteins expressed on the surface of exosomes.

In some embodiments, exosomes derived from a tissue of interest are selected for prior to detecting the RNA. For example, an antibody, such as a monoclonal or polyclonal antibody, or a peptide ligand, is used to select for a tissue specific marker on the surface of an exosome, such as to purify the exosome from a specific tissue, such as from the liver. For example, SRBI is a hepatocyte marker that can be used to select for exosomes from liver. The exosomes in the biological samples featured in the invention may be purified by other methods known in the art. For example, differential centrifugation can be used to purify exosomes (see, e.g., Raposo et al., J Exp Med (1996) 183:1161-1172). Other methods include anion exchange chromatography, gel permeation chromatography (e.g., U.S. Pat. Nos. 6,899,863 and 6,812,023), sucrose density gradients, organelle electrophoresis (e.g., U.S. Pat. No. 7,198,923) magnetic activated cell sorting (MACS) (Taylor and Gercel-Taylor, Gynecology Oncology (2008) 110(1): 13-21) and nanomembrane ultrafiltration concentrators (Cheruvanky et al., Am J Physiol Renal Physiol (2007) 292(5):F1657-61). Other methods of purifying exosomes are described in Gibbings et al. (U.S. 2011/0177054), which is hereby incorporated by reference in its entirety.

Exosomes in a biological sample may be enriched for those originating from a specific cell type, such as, for example, liver, lung, stomach, intestine, bladder, kidney, ovary, testis, skin, colorectal, breast, prostate, brain, spinal cord, choroid plexus, peripheral neurons or nerve, esophagus, adrenal gland, spleen, placenta, tumors, cancer lesions, or fetus cells. Because the exosomes carry surface molecules such as antigens from their donor cells, surface molecules may be used to identify exosomes from a specific donor cell type (Al-Nedawi et al., Nat Cell Biol (2008) 10:619-624; Taylor and Gercel-Taylor, Gynecology Oncology (2008) 110(1): 13-21). In this way, exosomes originating from distinct cell populations can be analyzed for their nucleic acid (e.g., RNA) content. For example, tumor (malignant and non-malignant) exosomes carry tumor-associated surface antigens and can be detected via tumor-associated surface antigens.

The purification of exosomes from specific cell types can be accomplished, for example, by using antibodies, aptamers, aptamer analogs or molecularly imprinted polymers specific for a desired surface antigen. In one embodiment, the surface antigen is specific for a cancer type. In another embodiment, the surface antigen is specific for a cell type that is not necessarily cancerous. One example of a method of exosome separation based on cell surface antigen is provided in U.S. Pat. No. 7,198,923. As described in, e.g., U.S. Pat. Nos. 5,840,867 and 5,582,981, and WO/2003/050290, aptamers and their analogs specifically bind surface molecules and can be used as a separation tool for retrieving cell type-specific exosomes. Molecularly imprinted polymers also specifically recognize surface molecules as described in, e.g., U.S. Pat. Nos. 6,525,154; 7,332,553; and 7,384,589 and are a tool for retrieving and isolating cell type-specific exosomes. Exosomes can also be identified and purified from a biological sample by a microchip technology that uses a microfluidic platform to efficiently and selectively separate tumor derived microvesicles (Nagrath et al., Nature (2007) 450:1235-9).

It may be beneficial or otherwise desirable to extract the nucleic acid (e.g., RNA) from a biological sample prior to the analysis. Nucleic acid molecules can be isolated from the sample using any number of procedures, which are well-known in the art, the particular isolation procedure chosen being appropriate for the particular biological sample. In some instances, with some techniques, it may also be possible to analyze the nucleic acid without extraction from the biological sample.

In one embodiment, the extracted nucleic acids, including RNA, are analyzed directly without an amplification step. Direct analysis may be performed with different methods including, but not limited to branched DNA (bDNA) or nanostring technology. NanoString technology enables identification and quantification of individual target molecules in a biological sample by attaching a color-coded fluorescent reporter to each target molecule. This approach is similar to the concept of measuring inventory by scanning barcodes. Reporters can be made with hundreds or even thousands of different codes allowing for highly multiplexed analysis. The technology is described by Geiss et al. (Geiss et al., Nat Biotech (2008) 26:317-325) and is incorporated herein by reference in its entirety.

“Microvesicles” are membrane fragments that are shed or pinched-off from the plasma membrane. Microvesicles are generally thought to be larger than exosomes (e.g., 100 nm to 1000 nm), but their features and biogenesis are believed to be similar RNAs, including mRNA and miRNA are present in microvesicles, which are released in a regulated fashion similar to that of exosomes. In some embodiments, a fraction of the LECT2 RNA or cleavage product thereof in the biological sample is present in microvesicles. Microvesicles are typically not isolated from the biological sample prior to detection of the LECT2 RNA.

In some embodiments, the LECT2 RNA or a cleavage product thereof is isolated from a biological sample comprising exosomes and microvesicles (e.g., extracellular vesicles).

In some embodiments, exosomes are separated from microvesicles, e.g., by size using, e.g., density gradient centrifugation or other methods known in the art.

In certain embodiments, the biological sample (e.g., bodily fluid sample) comprises circulating extracellular RNA.

### Isolation and Amplification of LECT2 RNA

RNA obtained from a biological sample can be isolated by any standard means known to a skilled artisan. Standard methods of RNA isolation, as well as recombinant nucleic acid methods used herein generally, are described in Sambrook et al., Molecular Biology: A laboratory Approach, Cold Spring Harbor, N.Y. 1989; Ausubel, et al., Current protocols in Molecular Biology, Greene Publishing, Y, 1995.

Nucleic acids can be recovered from the biological samples by extraction with an organic solvent, chloroform extraction, phenol-chloroform extraction, precipitation with ethanol, isopropanol or any other lower alcohol, by chromatography including ion exchange chromatography, size exclusion chromatography, silica gel chromatography and reversed phase chromatography, or by electrophoretic methods, including polyacrylamide gel electrophoresis and agarose gel electrophoresis, as will be apparent to one of skill in the art.

In one embodiment, a LECT2 RNA is isolated from the biological sample using phenol chloroform extraction. In another embodiment, the RNA is isolated from the biological sample using TRIZOL™ reagent (available from INVITROGEN™, Carlsbad, Calif.).

Following isolation, RNA can optionally be purified by techniques which are well known in the art. In one embodiment, purification results in RNA that is substantially free from contaminating DNA or proteins. Further purification can be accomplished by any of the aforementioned techniques for nucleic acid recovery. RNA can be purified by precipitation using a lower alcohol, especially with ethanol or with isopropanol. Precipitation can be carried out in the presence of a carrier such as glycogen that facilitates precipitation.

RNA from the biological sample can be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19:4967 (1991); Eckert et al., PCR Methods and Applications 1:17 (1991); PCR (Eds. McPherson et al., IRL Press, Oxford); and U.S. Pat. Nos. 4,683,202; 4,683,195; 4,800,159; 4,965,188; and 5,333,675, each of which is incorporated herein by reference in their entirety. The sample can be amplified on an array. See, for example, U.S. Pat. No. 6,300,070 and U.S. patent application Ser. No. 09/513,300, which are incorporated herein by reference.

Other suitable amplification methods include the ligase chain reaction (LCR) (e.g., Wu and Wallace, Genomics 4:560 (1989), Landegren et al., Science 241:1077 (1988) and Barringer et al. Gene 89:117 (1990)), transcription amplification (Kwoh et al., Proc. Natl. Acad. Sci. USA 86:1173 (1989) and WO88/10315), self-sustained sequence replication (Guatelli et al., Proc. Nat. Acad. Sci. USA, 87:1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909; 5,861,245) and nucleic acid based sequence amplification (NABSA). (See U.S. Pat. Nos. 5,409,818; 5,554,517; and 6,063,603; each of which is incorporated herein by reference). Other amplification methods that can be used are described in, U.S. Pat. Nos. 5,242,794; 5,494,810; 4,988,617; and 6,582,938, each of which is incorporated herein by reference in its entirety.

RNA isolated by the method of the present invention can include messenger RNA (mRNA), transfer RNA (tRNA), and ribosomal RNA (rRNA). In one embodiment, the RNA is mRNA. In another embodiment, the RNA is an mRNA cleavage product. In another embodiment, the RNA is a RISC-cleavage product.

Typically, isolation of the RNA requires a process that includes a physical change in a physical substance, e.g., a starting material, such as biological sample and the contents of the sample (e.g., the RNA). Exemplary changes include making a physical entity from two or more starting materials (e.g., by polymerase chain reaction), shearing or fragmenting a substance, separating or purifying a substance, combining two or more separate entities into a mixture, or performing a chemical reaction that includes breaking or forming a covalent or non-covalent bond.

### Methods for Detecting LECT2 RNA or a Cleavage Product Thereof

Methods for assessing RNA levels, e.g., mRNA levels, are well known to those skilled in the art. Detection of RNA transcripts can be accomplished using known amplification methods. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribed mRNA into cDNA followed by symmetric gap ligase chain reaction (RT-AGLCR) as described by R. L. Marshall, et al., PCR Methods and Applications 4: 80-84 (1994).

In one embodiment, gene expression is measured using quantitative real time PCR. Quantitative real-time PCR refers to a polymerase chain reaction which is monitored, usually by fluorescence, over time during the amplification process, to measure a parameter related to the extent of amplification of a particular sequence. The amount of fluorescence released during the amplification cycle is proportional to the amount of product amplified in each PCR cycle.

Hybridization methods can also be employed, for example, a radioactively or fluorescently labeled antisense RNA probe is hybridized to isolated RNA, washed, cleaved with RNase and visualized. Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described, for example, in U.S. Pat. Nos. 5,871,928; 5,874,219; 6,045,996 and 6,386,749; 6,391,623 each of which are incorporated herein by reference.

RNA expression, including mRNA expression, can be detected on a DNA array, chip or a microarray. Oligonucleotides corresponding to a target RNA are immobilized on a chip which is then hybridized with labeled RNA of a biological sample obtained from a subject. A positive hybridization signal is obtained with the sample containing transcripts of the target gene. Methods of preparing DNA arrays and their use are well known in the art. (See, for example, U.S. Pat. Nos. 6,618,6796; 6,379,897; 6,664,377; 6,451,536; 548,257; U.S. 20030157485 and Schena et al. 1995 Science 20:467-470; Gerhold et al. 1999 Trends in Biochem. Sci. 24, 168-173; and Lennon et al. 2000 Drug discovery Today 5: 59-65, which are herein incorporated by reference in their entirety). Serial Analysis of Gene Expression (SAGE) can also be performed (See for example U.S. Patent Application 20030215858).

5′ RACE (“Rapid Amplification of cDNA Ends”) techniques can be employed in detection of a cleavage product and can be used e.g., to obtain the full-length RNA associated with the cleavage product. 5′ RACE techniques typically involve producing a cDNA copy of the RNA sequence of interest using reverse transcription followed by PCR amplification of the cDNA copies. The amplified cDNA copies are sequenced can be mapped to a full-length mRNA sequence. Methods and kits for performing 5′ RACE are known to those of skill in the art.

Typically, detecting or analyzing or measuring RNA (e.g., RNA levels) requires a process that includes a physical change in a physical substance, e.g., a starting material, such as isolated RNA. Exemplary changes include making a physical entity from two or more starting materials (e.g., by polymerase chain reaction), shearing or fragmenting a substance, separating or purifying a substance, combining two or more separate entities into a mixture, or performing a chemical reaction that includes breaking or forming a covalent or non-covalent bond. Analyzing a sample can include performing an analytical process which includes a physical change in a substance, e.g., an RNA sample, an analyte, or a reagent (sometimes referred to herein as “physical analysis”), performing an analytical method, e.g., a method which includes one or more of the following: combining a nucleic acid, an analyte, or fragment or other derivative thereof, with another substance, e.g., a buffer, solvent, enzyme or reactant; changing the structure of a nucleic acid, an analyte, or a fragment or other derivative thereof, e.g., by breaking or forming a covalent or non-covalent bond, between a first and a second atom of the nucleic acid or analyte; or changing the structure of a reagent, or a fragment or other derivative thereof, e.g., by breaking or forming a covalent or non-covalent bond, between a first and a second atom of the reagent; or separating or purifying a substance, e.g., a nucleic acid an analyte, or a fragment or other derivative thereof, from another substance.

### Reference Samples

A “reference sample” as used herein is, for example, a biological sample (e.g., a bodily fluid sample) obtained from a subject who does not have a LECT2-associated disorder (e.g., ALECT2), e.g., a healthy subject, or a subject having a LECT2-associated disorder who has not received a nucleic acid agent for treating the LECT2-associated disorder, or obtained from a patient prior to receiving a nucleic acid agent for treating the LECT2-associated disorder.

For example, the results of an assay described herein, e.g., to measure the level of LECT2 RNA in a biological sample (e.g., a bodily fluid sample described herein) obtained from a subject having a LECT2-associated disorder described herein (e.g., ALECT2), can be compared to the level of LECT2 RNA in the same type of biological sample obtained from a subject who does not have the LECT2-associated disorder, e.g., a healthy subject. In some embodiments, the level of LECT2 RNA may be absent or so low as to be undetectable in the biological sample (e.g., a urine or serum sample) obtained from a subject who does not have the LECT2-associated disorder, e.g., a healthy subject.

As another example, the results of an assay described herein, e.g., to measure the level of LECT2 RNA or a cleavage product thereof in a biological sample (e.g., a bodily fluid sample described herein) obtained from a subject, following administration of a nucleic agent (e.g., a dsRNA or antisense polynucleotide agent) or to determine the activity of a nucleic acid agent on target RNA degradation, can be compared to the level of LECT2 RNA in the same type of biological sample obtained from the subject prior to receiving the nucleic acid agent. In some embodiments, the LECT2 RNA levels measured (e.g., at a first and second time point) can be compared to the RNA levels of a gene that is not expected to vary with the nucleic acid agent (e.g., dsRNA or antisense polynucleotide agent) treatment (e.g., a housekeeping gene or non-targeted tissue-specific gene). In one embodiment, the LECT2 RNA levels in a sample following administration of a nucleic acid agent (e.g., a dsRNA or antisense polynucleotide agent) can be compared to levels of a ubiquitous control RNA (e.g., GAPDH, β-actin or ribosomal (e.g., 18S)) before and after administration of the nucleic acid agent. In another embodiment, the LECT2 RNA levels in a sample following administration of a nucleic acid agent (e.g., a dsRNA or antisense polynucleotide agent) can be compared to levels of a non-target tissue-specific RNA before and after administration of the nucleic acid agent. For example, levels of a non-target liver RNA, e.g., alpha antitrypsin (AAT), can be compared to LECT2 target RNA levels following administration of a nucleic acid agent (e.g., a dsRNA or antisense polynucleotide agent) that targets LECT2. In some embodiments, the LECT2 RNA levels are normalized to the RNA levels of a gene that is not expected to vary with the nucleic acid agent (e.g., dsRNA or antisense polynucleotide agent) treatment.

In some embodiments, when comparing RNA levels from different samples, the LECT2 RNA levels can be normalized by comparison to RNA levels of an endogenous gene (e.g., a housekeeping gene, e.g., GAPDH, β-actin, or ribosomal (e.g., 18S)) from the same sample.

In some embodiments, the reference sample is a population standard determined, e.g., by averaging the LECT2 RNA levels measured in a biological sample (e.g., a bodily fluid sample described herein) among individuals in a normal population and/or in a diseased population. The diseased population can be further separated into untreated and nucleic acid agent treated groups for measuring target RNA levels and determining the population standard. Such population standards are useful in determining an acceptable range or threshold value of LECT2 RNA levels that can be used, e.g., in the clinic for comparison with an individual's LECT2 RNA levels. Thus, in some embodiments the reference sample is a number (e.g., a threshold value, a cut-off value, an acceptable range, etc.).

### Kits

Components for the methods and assays discussed herein can be provided in a kit. The kit can include, for example, primers, e.g., to reverse-transcribe and/or amplify a target RNA from a biological sample. The kit can include, for example, primers for 5′ RACE amplification of an RNA.

In one embodiment, the kit provides one or more primer pairs, each pair capable of amplifying a desired target transcript thereby providing a kit for analysis of expression of one or more targets in a biological sample in one reaction or several parallel reactions. Primers in the kits may be labeled, for example fluorescently labeled, to facilitate detection of the amplification products and consequent analysis of the expression levels of the target RNA or cleavage product thereof.

In one embodiment, more than one target gene can be detected in one analysis. A combination kit will therefore comprise primers capable of amplifying different target RNA. The primers may be differentially labeled, for example using different fluorescent labels, so as to differentiate between the target RNAs to be detected.

The primers contained within the kit can be designed for 5′ RACE, reverse transcription or TAQMAN® quantitative PCR using a sequence for Leukocyte cell-derived chemotaxin-2 (LECT2) (GenBank Accession No. NM_002302.2).

In addition to the one or more primers, the kit can include other ingredients, such as a solvent or buffer, a stabilizer, or a preservative. The agent can be provided in any form, e.g., liquid, dried or lyophilized form, and in substantially pure and/or sterile form. When the agents are provided in a liquid solution, the liquid solution is, for example, an aqueous solution. When the agents are provided as a dried form, reconstitution generally is by the addition of a suitable solvent. The solvent, e.g., sterile water or buffer, can optionally be provided in the kit.

The kit can also include instructions, such as for amplification protocols and analysis of the results. The instructions may also specify that the person have been administered a nucleic acid agent (e.g., a dsRNA or antisense polynucleotide agent). The kit may alternatively also comprise buffers, enzymes, and containers for performing the amplification and analysis of the amplification products. The informational material of the kits is not limited in its form. For example, the informational material can include information about how to perform an assay, concentrations of required reagents, date of expiration, batch or production site information, and so forth. In one embodiment, the informational material relates to methods of obtaining a biological sample, and isolating and measuring levels of a target RNA or cleavage product thereof. The information can be provided in a variety of formats, including printed text, computer readable material, video recording, or audio recording, or information that provides a link or address (e.g., a URL) to substantive material.

In one embodiment, the kit can include informational material for performing and interpreting the assay. In another embodiment, the kit can provide guidance as to where to report the results of the assay, e.g., to a research facility, treatment center or healthcare provider. The kit can include forms for reporting the results of an assay described herein, and address and contact information regarding where to send such forms or other related information; or a URL (Uniform Resource Locator) address for reporting the results in an online database or an online application (e.g., an app). In another embodiment, the informational material can include guidance regarding whether a patient should receive treatment or continue to receive treatment with an iRNA agent, depending on the results of the assay.

The kit may also include reagents for isolating RNA from a biological sample including, for example, phenol/chloroform or TRIzol™ reagent. In some embodiments, reagents that increase RNA yield during isolation are also included in the kit. In one embodiment, a reagent to increase RNA yield is lithium chloride, e.g., which can be added to the sample at a final concentration of 0.1-5 M (e.g., 0.5, 1, 1.5, 2, 2.5 or 3M). The kit can further include a reagent such as glycogen that acts as a co-precipitant to help pellet small volumes of RNA. The kit may include reagents to inhibit RNase activity, or detergents to lyse RNA carrying vesicles (exosomes).

The kit can further include an agent to inhibit RNase activity in the biological sample or to enhance RNA precipitation. For example, the kit can include RNase inhibitor or EDTA to inhibit RNase activity, and/or can include PEG or lithium chloride to enhance RNA precipitation.

The kit may also be a component of a screening, diagnostic or prognostic kit comprising other tools such as DNA microarrays. The kit can also include one or more control templates, such as LECT2 RNA isolated from a reference sample (e.g., a reference sample described herein) or a recombinant LECT2 RNA. In some embodiments, a biological sample isolated from the subject prior to being administered a nucleic acid agent (e.g., a dsRNA or antisense polynucleotide agent) can be used as the control.

The kit can include one or more containers for the composition or compositions containing the agents. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit, e.g., a unit that includes primers for RT-PCR, or primers that target more than one RNA. In one example, the kit includes a plurality of syringes, ampoules, foil packets, blister packs, or medical devices, e.g., each containing a single combination unit, such as for obtaining a biological sample, and isolating and detecting target RNA from the sample. The containers of the kits can be air tight, waterproof (e.g., impermeable to changes in moisture or evaporation), and/or light-tight.

### III. iRNA Agents

The nucleic acid agents described herein include, e.g., iRNAs. The iRNAs described herein can modulate the expression of a LECT2 gene. In certain embodiments, expression of a LECT2 gene is reduced or inhibited using a LECT2-specific iRNA. Such inhibition can be useful in treating disorders related to LECT2 expression, such as amyloidosis, e.g. a LECT2 amyloidosis (ALECT2).

In some embodiments, the iRNA agent effects the RNA-induced silencing complex (RISC)-mediated cleavage of RNA transcripts of the LECT2 gene, such as in a cell or in a subject (e.g., in a mammal, such as a human subject). In some embodiments, the iRNA agent is used in a method of treating a disorder related to expression of a LECT2 gene, such as a LECT2 amyloidosis.

In some embodiments, the LECT2 amyloidosis is a renal amyloidosis. In some embodiments, the LECT2 amyloidosis involves amyloid deposition in the kidney. In some embodiments, LECT2 amyloidosis is associated with renal disease (e.g., renal insufficiency or nephrotic syndrome). In some embodiments, the amyloidosis is associated with proteinuria. In some embodiments, proteinuria is absent. In some embodiments, the LECT2 amyloidosis is a hepatic amyloidosis. In some embodiments, the LECT2 amyloidosis involves amyloid deposition in the liver. In some embodiments, the LECT2 amyloidosis is associated with inflammation in the liver (e.g., hepatitis, e.g., chronic hepatitis).

In some embodiments, the subject is of Mexican descent (e.g., a Mexican American). In some embodiments, the subject carries the G allele of the LECT2 gene that encodes valine at position 40 in the mature protein (or amino acid 58 in the unprocessed protein). In some embodiments, the subject is homozygous for the G allele (G/G genotype). In some embodiments, a LECT2 protein expressed in the subject has valine at position 40 in the mature protein (or at amino acid 58 in the unprocessed protein).

In one embodiment, the iRNA inhibits LECT2 expression in a cell or mammal. In some embodiments, the method is effective to inhibit amyloid deposition (e.g., by preventing amyloid deposition or reducing amyloid deposition, e.g., by reducing size, number, or extent of amyloid deposits) or symptoms associated with amyloid deposition.

In some embodiments, the iRNA (e.g., dsRNA) includes an RNA strand (the antisense strand) having a region, e.g., a region that is 30 nucleotides or less, generally 19-24 nucleotides in length, that is substantially complementary to at least part of an mRNA transcript of a LECT2 gene (e.g., a mouse or human LECT2 gene) (also referred to herein as a “LECT2-specific iRNA”).

In some embodiments, the LECT2 mRNA transcript is a human LECT2 mRNA transcript, e.g., SEQ ID NO: 1. In some embodiments, the LECT2 mRNA transcript has an A to G substitution at nucleotide position 373 of SEQ ID NO: 1. In some embodiments, the mRNA transcript encodes valine at position 40 in the mature LECT2 protein (or amino acid 58 in the unprocessed protein). In some embodiments, the mRNA transcript encodes isoleucine at position 40 in the mature LECT2 protein (or amino acid 58 in the unprocessed protein).

In some embodiments, the iRNA (e.g., dsRNA) comprises an antisense strand having a region that is substantially complementary to a region of a human LECT2 mRNA. In some embodiments, the human LECT2 mRNA has the sequence of NM_002302.2 (SEQ ID NO: 1). In some embodiments, the human LECT2 mRNA has an A to G substitution at nucleotide position 373 of SEQ ID NO: 1.

In other embodiments, an iRNA encompasses a dsRNA having an RNA strand (the antisense strand) having a region that is substantially complementary to a portion of a LECT2 mRNA. In one embodiment, the iRNA encompasses a dsRNA having an RNA strand (the antisense strand) having a region that is substantially complementary to a portion of a LECT2 mRNA, e.g., a human LECT2 mRNA (e.g., a human LECT2 mRNA as provided in NM_002302.2 (SEQ ID NO: 1) or having an A to G substitution at nucleotide position 373 of SEQ ID NO: 1).

In one embodiment, an iRNA for inhibiting expression of a LECT2 gene includes at least two sequences that are complementary to each other. The iRNA includes a sense strand having a first sequence and an antisense strand having a second sequence. The antisense strand includes a nucleotide sequence that is substantially complementary to at least part of an mRNA encoding a LECT2 transcript, and the region of complementarity is 30 nucleotides or less, and at least 15 nucleotides in length. Generally, the iRNA is 19 to 24 nucleotides in length.

In some embodiments, the iRNA is 19-21 nucleotides in length. In some embodiments, the iRNA is 19-21 nucleotides in length and is in a lipid formulation, e.g. a lipid nanoparticle (LNP) formulation (e.g., an LNP11 formulation). In one embodiment, the iRNA targeting LECT2 is formulated in a stable nucleic acid lipid particle (SNALP).

In some embodiments, the iRNA is 21-23 nucleotides in length. In some embodiments, the iRNA is 21-23 nucleotides in length and is in the form of a conjugate, e.g., conjugated to one or more GalNAc derivatives as described herein.

In some embodiments, the iRNA is from about 15 to about 25 nucleotides in length, and in other embodiments the iRNA is from about 25 to about 30 nucleotides in length. An iRNA targeting LECT2, upon contact with a cell expressing LECT2, inhibits the expression of a LECT2 gene (e.g., by at least 10%, at least 20%, at least 25%, at least 30%, at least 35% or at least 40%, at least 50%, at least 60%, at least 70%, or at least 80%) when assayed by a method known in the art or as described herein.

In one embodiment, the iRNA (e.g., dsRNA) comprises or consists of a first sequence of a dsRNA that is selected from the group consisting of the sense sequences of Tables 2-3, 5-6 and 9-10 of WO2015/050990 and a second sequence that is selected from the group consisting of the corresponding antisense sequences of Tables 2-3, 5-6 and 9-10 of WO2015/050990.

In some embodiments, the iRNA (e.g., dsRNA) comprises or consists of a sense and/or antisense sequence selected from those provided in Table 2-3, 5-6 and 9-10 of WO2015/050990.

In some embodiments, the iRNA (e.g., dsRNA) comprises or consists of a first sequence of a dsRNA that is selected from the group consisting of the sense sequences of Table 2 and a second sequence that is selected from the group consisting of the corresponding antisense sequences of Table 2.

In some embodiments, the iRNA (e.g., dsRNA) comprises or consists of a sense and/or antisense sequence selected from those provided in Table 2.

In some embodiments, the iRNA (e.g., dsRNA) comprises or consists of a first sequence of a dsRNA that is selected from the group consisting of the sense sequences of Table 3 and a second sequence that is selected from the group consisting of the corresponding antisense sequences of Table 3.

In some embodiments, the iRNA (e.g., dsRNA) comprises or consists of a sense and/or antisense sequence selected from those provided in Table 3.

In some embodiments, the iRNA (e.g., dsRNA) comprises an antisense sequence that comprises at least 15, 16, 17, 18, 19, 20, 21, 22, or 23 contiguous of an antisense sequence provided in Table 2 and a sense sequence that comprises at least 15, 16, 17, 18, 19, 20, or 21 contiguous of a corresponding sense sequence provided in Table 2.

In some embodiments, the iRNA (e.g., dsRNA) comprises an antisense sequence that comprises at least 15, 16, 17, 18, 19, 20, 21, 22, or 23 contiguous of an antisense sequence provided in Table 3 and a sense sequence that comprises at least 15, 16, 17, 18, 19, 20, or 21 contiguous of a corresponding sense sequence provided in Table 3.

TABLE 2
Modified LECT2 siRNA Sequences

| | Sense | | SEQ | | | SEQ | Antisense |
|---|---|---|---|---|---|---|---|
| Duplex | Oligo | | ID | Antisense | | ID | position in |
| Name | Name | Sense Sequence | NO | Oligo Name | Antisense Sequence | NO | NM_002302 |
| AD-61249 | A-122943 | UfsasGfcUfaGfgAfAfG | 497 | A-122944 | usGfsaAfuGfaAfuAfc | 498 | 106-128 |
| | | fuAfuUfcAfuUfcAfL96 | | | uuCfcUfaGfcUfasusu | | |
| AD-61260 | A-122931 | AfsgsCfuAfgGfaAfGfU | 499 | A-122932 | usUfsgAfaUfgAfaUfa | 500 | 107-129 |
| | | faUfuCfaUfuCfaAfL96 | | | cuUfcCfuAfgCfusasu | | |
| AD-61254 | A-122929 | AfsusUfcAfuUfcAfAfA | 501 | A-122930 | asAfsuAfuUfcAfaGfu | 502 | 118-140 |
| | | fcUfuGfaAfuAfuUfL96 | | | uuGfaAfuGfaAfusasc | | |
| AD-61243 | A-122941 | UfsusCfaAfaCfuUfGfA | 503 | A-122942 | usGfsaAfgAfaUfaUfu | 504 | 123-145 |
| | | faUfaUfuCfuUfcAfL96 | | | caAfgUfuUfgAfasusg | | |
| AD-61261 | A-122947 | UfsusGfaAfuAfuUfCfU | 505 | A-122948 | csUfscUfcUfuUfgAfa | 506 | 130-152 |
| | | fuCfaAfaGfaGfaGfL96 | | | gaAfuAfuUfcAfasgsu | | |
| AD-61242 | A-122925 | UfsusGfaAfuAfuUfCfU | 507 | A-122926 | usUfscUfcUfuUfgAfa | 508 | 130-152_G21A |
| | | fuCfaAfaGfaGfaAfL96 | | | gaAfuAfuUfcAfasgsu | | |
| AD-61248 | A-122927 | GfscsUfaAfuAfuAfUfG | 509 | A-122928 | csUfsuGfcCfaGfcAfc | 510 | 263-285 |
| | | fuGfcUfgGfcAfaGfL96 | | | auAfuAfuUfaGfcscsc | | |
| AD-61277 | A-122921 | GfscsUfaAfuAfuAfUfG | 511 | A-122922 | usUfsuGfcCfaGfcAfc | 512 | 263-285_G21A |
| | | fuGfcUfgGfcAfaAfL96 | | | auAfuAfuUfaGfcscsc | | |
| AD-61253 | A-122913 | CfsasUfgGfcUfgUfGfG | 513 | A-122914 | asGfsaGfuAfcUfgUfc | 514 | 314-336 |
| | | faCfaGfuAfcUfcUfL96 | | | caCfaGfcCfaUfgsgsc | | |
| AD-61259 | A-122915 | UfsgsGfcUfgUfgGfAfC | 515 | A-122916 | gsCfsaGfaGfuAfcUfg | 516 | 316-338 |
| | | faGfuAfcUfcUfgCfL96 | | | ucCfaCfaGfcCfasusg | | |
| AD-61271 | A-122919 | UfsgsGfcUfgUfgGfAfC | 517 | A-122920 | usCfsaGfaGfuAfcUfg | 518 | 316-338_C21A |
| | | faGfuAfcUfcUfgAfL96 | | | ucCfaCfaGfcCfasusg | | |
| AD-61239 | A-122877 | GfsgsCfuGfuGfgAfCfA | 519 | A-122878 | asGfscAfgAfgUfaCfu | 520 | 317-339 |
| | | fgUfaCfuCfuGfcUfL96 | | | guCfcAfcAfgCfcsasu | | |
| AD-61257 | A-122883 | GfscsUfgUfgGfaCfAfG | 521 | A-122884 | gsAfsgCfaGfaGfuAfc | 522 | 318-340 |
| | | fuAfcUfcUfgCfuCfL96 | | | ugUfcCfaCfaGfcscsa | | |
| AD-61246 | A-122895 | GfscsUfgUfgGfaCfAfG | 523 | A-122896 | usAfsgCfaGfaGfuAfc | 524 | 318-340_C21A |
| | | fuAfcUfcUfgCfuAfL96 | | | ugUfcCfaCfaGfcscsa | | |
| AD-61245 | A-122879 | CfsusGfuGfgAfcAfGfU | 525 | A-122880 | usGfsaGfcAfgAfgUfa | 526 | 319-341 |
| | | faCfuCfuGfcUfcAfL96 | | | cuGfuCfcAfcAfgscsc | | |
| AD-61268 | A-122871 | UfsgsUfgGfaCfaGfUfA | 527 | A-122872 | usUfsgAfgCfaGfaGfu | 528 | 320-342 |
| | | fcUfcUfgCfuCfaAfL96 | | | acUfgUfcCfaCfasgsc | | |
| AD-61251 | A-122881 | GfsusGfgAfcAfgUfAfC | 529 | A-122882 | usUfsuGfaGfcAfgAfg | 530 | 321-343 |
| | | fuCfuGfcUfcAfaAfL96 | | | uaCfuGfuCfcAfcsasg | | |
| AD-61281 | A-122891 | UfsgsGfaCfaGfuAfCfU | 531 | A-122892 | csUfsuUfgAfgCfaGfa | 532 | 322-344 |
| | | fcUfgCfuCfaAfaGfL96 | | | guAfcUfgUfcCfascsa | | |
| AD-61258 | A-122899 | UfsgsGfaCfaGfuAfCfU | 533 | A-122900 | asUfsuUfgAfgCfaGfa | 534 | 322-344_G21U |
| | | fcUfgCfuCfaAfaUfL96 | | | guAfcUfgUfcCfascsa | | |
| AD-61275 | A-122889 | GfsgsAfcAfgUfaCfUfC | 535 | A-122890 | usCfsuUfuGfaGfcAfg | 536 | 323-345 |
| | | fuGfcUfcAfaAfgAfL96 | | | agUfaCfuGfuCfcsasc | | |
| AD-61269 | A-122887 | GfsasCfaGfuAfcUfCfU | 537 | A-122888 | usUfscUfuUfgAfgCfa | 538 | 324-346 |
| | | fgCfuCfaAfaGfaAfL96 | | | gaGfuAfcUfgUfcscsa | | |
| AD-61279 | A-122955 | UfsgsUfgGfgCfcAfGfG | 539 | A-122956 | usAfsaGfgUfuUfcUfc | 540 | 421-443 |
| | | faGfaAfaCfcUfuAfL96 | | | cuGfgCfcCfaCfasasu | | |
| AD-61278 | A-122937 | CfscsAfgGfaGfaAfAfC | 541 | A-122938 | usUfsuUfgAfuAfaGfg | 542 | 427-449 |
| | | fcUfuAfuCfaAfaAfL96 | | | uuUfcUfcCfuGfgscsc | | |
| AD-61247 | A-122911 | UfsgsUfuCfuAfcAfUfU | 543 | A-122912 | usAfsaUfuGfgCfuUfa | 544 | 504-526 |
| | | faAfgCfcAfaUfuAfL96 | | | auGfuAfgAfaCfasusu | | |
| AD-61241 | A-122909 | GfsusUfcUfaCfaUfUfA | 545 | A-122910 | usUfsaAfuUfgGfcUfu | 546 | 505-527 |
| | | faGfcCfaAfuUfaAfL96 | | | aaUfgUfaGfaAfcsasu | | |
| AD-61282 | A-122907 | UfsusCfuAfcAfuUfAfA | 547 | A-122908 | csUfsuAfaUfuGfgCfu | 548 | 506-528 |
| | | fgCfcAfaUfuAfaGfL96 | | | uaAfuGfuAfgAfascsa | | |
| AD-61265 | A-122917 | UfsusCfuAfcAfuUfAfA | 549 | A-122918 | usUfsuAfaUfuGfgCfu | 550 | 506-528_G21A |
| | | fgCfcAfaUfuAfaAfL96 | | | uaAfuGfuAfgAfascsa | | |
| AD-61276 | A-122905 | UfscsUfaCfaUfuAfAfG | 551 | A-122906 | asCfsuUfaAfuUfgGfc | 552 | 507-529 |
| | | fcCfaAfuUfaAfgUfL96 | | | uuAfaUfgUfaGfasasc | | |
| AD-61250 | A-122865 | CfsusAfcAfuUfaAfGfC | 553 | A-122866 | usAfscUfuAfaUfuGfg | 554 | 508-530 |
| | | fcAfaUfuAfaGfuAfL96 | | | cuUfaAfuGfuAfgsasa | | |
| AD-61256 | A-122867 | UfsasCfaUfuAfaGfCfC | 555 | A-122868 | asUfsaCfuUfaAfuUfg | 556 | 509-531 |
| | | faAfuUfaAfgUfaUfL96 | | | gcUfuAfaUfgUfasgsa | | |
| AD-61262 | A-122869 | AfscsAfuUfaAfgCfCfA | 557 | A-122870 | usAfsuAfcUfuAfaUfu | 558 | 510-532 |
| | | faUfuAfaGfuAfuAfL96 | | | ggCfuUfaAfuGfusasg | | |
| AD-61244 | A-122863 | CfsasUfuAfaGfcCfAfA | 559 | A-122864 | usUfsaUfaCfuUfaAfu | 560 | 511-533 |
| | | fuUfaAfgUfaUfaAfL96 | | | ugGfcUfuAfaUfgsusa | | |
| AD-61238 | A-122861 | AfsusUfaAfgCfcAfAfU | 561 | A-122862 | usUfsuAfuAfcUfuAfa | 562 | 512-534 |
| | | fuAfaGfuAfuAfaAfL96 | | | uuGfgCfuUfaAfusgsu | | |
| AD-61280 | A-122875 | UfsusAfaGfcCfaAfUfU | 563 | A-122876 | csUfsuUfaUfaCfuUfa | 564 | 513-535 |
| | | faAfgUfaUfaAfaGfL96 | | | auUfgGfcUfuAfasusg | | |
| AD-61240 | A-122893 | UfsusAfaGfcCfaAfUfU | 565 | A-122894 | asUfsuUfaUfaCfuUfa | 566 | 513-535_G21U |
| | | faAfgUfaUfaAfaUfL96 | | | auUfgGfcUfuAfasusg | | |
| AD-61263 | A-122885 | UfsasAfgCfcAfaUfUfA | 567 | A-122886 | csCfsuUfuAfuAfcUfu | 568 | 514-536 |
| | | faGfuAfuAfaAfgGfL96 | | | aaUfuGfgCfuUfasasu | | |
| AD-61252 | A-122897 | UfsasAfgCfcAfaUfUfA | 569 | A-122898 | usCfsuUfuAfuAfcUfu | 570 | 514-536_G21A |
| | | faGfuAfuAfaAfgAfL96 | | | aaUfuGfgCfuUfasasu | | |
| AD-61274 | A-122873 | AfsasGfcCfaAfuUfAfA | 571 | A-122874 | asCfscUfuUfaUfaCfu | 572 | 515-537 |
| | | fgUfaUfaAfaGfgUfL96 | | | uaAfuUfgGfcUfusasa | | |
| AD-61270 | A-122903 | AfsgsCfcAfaUfuAfAfG | 573 | A-122904 | gsAfscCfuUfuAfuAfc | 574 | 516-538 |
| | | fuAfuAfaAfgGfuCfL96 | | | uuAfaUfuGfgCfususa | | |
| AD-61264 | A-122901 | AfsgsCfcAfaUfuAfAfG | 575 | A-122902 | usAfscCfuUfuAfuAfc | 576 | 516-538_C21A |
| | | fuAfuAfaAfgGfuAfL96 | | | uuAfaUfuGfgCfususa | | |
| AD-61255 | A-122945 | GfscsCfcUfuGfcAfGfA | 577 | A-122946 | gsGfsaUfaAfaCfuUfu | 578 | 571-593 |
| | | faAfgUfuUfaUfcCfL96 | | | cuGfcAfaGfgGfcsasa | | |
| AD-61283 | A-122923 | GfscsCfcUfuGfcAfGfA | 579 | A-122924 | usGfsaUfaAfaCfuUfu | 580 | 571-593_C21A |
| | | faAfgUfuUfaUfcAfL96 | | | cuGfcAfaGfgGfcsasa | | |
| AD-61284 | A-122939 | CfscsCfuUfgCfaGfAfA | 581 | A-122940 | asGfsgAfuAfaAfcUfu | 582 | 572-594 |
| | | faGfuUfuAfuCfcUfL96 | | | ucUfgCfaAfgGfgscsa | | |
| AD-61267 | A-122950 | CfscsAfaUfgGfuCfAfG | 583 | A-122952 | usUfsuUfgAfaGfaUfc | 584 | 664-686 |
| | | faUfcUfuCfaAfaAfL96 | | | ugAfcCfaUfuGfgscsc | | |
| AD-61266 | A-122933 | CfsasAfuGfgUfcAfGfA | 585 | A-122934 | asUfsuUfuGfaAfgAfu | 586 | 665-687 |
| | | fuCfuUfcAfaAfaUfL96 | | | cuGfaCfcAfuUfgsgsc | | |
| AD-61273 | A-122953 | AfsasUfgGfuCfaGfAfU | 587 | A-122954 | usAfsuUfuUfgAfaGfa | 588 | 666-688 |
| | | fcUfuCfaAfaAfuAfL96 | | | ucUfgAfcCfaUfusgsg | | |
| AD-61272 | A-122935 | UfsgsGfuCfaGfaUfCfU | 589 | A-122936 | usUfsuAfuUfuUfgAfa | 590 | 668-690 |
| | | fuCfaAfaAfuAfaAfL96 | | | gaUfcUfgAfcCfasusu | | |

TABLE 3
Unmodified LECT2 siRNA Sequences

| | Sense | | SEQ | Sense | Antisense | | SEQ | Antisense |
|---|---|---|---|---|---|---|---|---|
| | Oligo | Sense | ID | Position in | Oligo | Antisense | ID | position in |
| Duplex Name | Name | Sequence | NO | NM_002302 | Name | Sequence | NO | NM_002302 |
| AD-61249U M | A-122943 | UAGCUAGGAAG | 591 | 108-128 | A-122944 | UGAAUGAAUACU | 592 | 106-128 |
| | | UAUUCAUUCA | | | | UCCUAGCUAUU | | |
| AD-61260 UM | A-122931 | AGCUAGGAAGU | 593 | 109-129 | A-122932 | UUGAAUGAAUAC | 594 | 107-129 |
| | | AUUCAUUCAA | | | | UUCCUAGCUAU | | |
| AD-61254 UM | A-122929 | AUUCAUUCAAA | 595 | 120-140 | A-122930 | AAUAUUCAAGUU | 596 | 118-140 |
| | | CUUGAAUAUU | | | | UGAAUGAAUAC | | |
| AD-61243 UM | A-122941 | UUCAAACUUGA | 597 | 125-145 | A-122942 | UGAAGAAUAUUC | 598 | 123-145 |
| | | AUAUUCUUCA | | | | AAGUUUGAAUG | | |
| AD-61261 UM | A-122947 | UUGAAUAUUCU | 599 | 132-152 | A-122948 | CUCUCUUUGAAG | 600 | 130-152 |
| | | UCAAAGAGAG | | | | AAUAUUCAAGU | | |
| AD-61242 UM | A-122925 | UUGAAUAUUCU | 601 | 132-152_G21A | A-122926 | UUCUCUUUGAAG | 602 | 130-152_G21A |
| | | UCAAAGAGAA | | | | AAUAUUCAAGU | | |
| AD-61248 UM | A-122927 | GCUAAUAUAUG | 603 | 265-285 | A-122928 | CUUGCCAGCACA | 604 | 263-285 |
| | | UGCUGGCAAG | | | | UAUAUUAGCCC | | |
| AD-61277 UM | A-122921 | GCUAAUAUAUG | 605 | 265-285_G21A | A-122922 | UUUGCCAGCACA | 606 | 263-285_G21A |
| | | UGCUGGCAAA | | | | UAUAUUAGCCC | | |
| AD-61253 UM | A-122913 | CAUGGCUGUGG | 607 | 316-336 | A-122914 | AGAGUACUGUCC | 608 | 314-336 |
| | | ACAGUACUCU | | | | ACAGCCAUGGC | | |
| AD-61259 UM | A-122915 | UGGCUGUGGAC | 609 | 318-338 | A-122916 | GCAGAGUACUGU | 610 | 316-338 |
| | | AGUACUCUGC | | | | CCACAGCCAUG | | |
| AD-61271 UM | A-122919 | UGGCUGUGGAC | 611 | 318-338_C21A | A-122920 | UCAGAGUACUGU | 612 | 316-338_C21A |
| | | AGUACUCUGA | | | | CCACAGCCAUG | | |
| AD-61239 UM | A-122877 | GGCUGUGGACA | 613 | 319-339 | A-122878 | AGCAGAGUACUG | 614 | 317-339 |
| | | GUACUCUGCU | | | | UCCACAGCCAU | | |
| AD-61257 UM | A-122883 | GCUGUGGACAG | 615 | 320-340 | A-122884 | GAGCAGAGUACU | 616 | 318-340 |
| | | UACUCUGCUC | | | | GUCCACAGCCA | | |
| AD-61246 UM | A-122895 | GCUGUGGACAG | 617 | 320-340_C21A | A-122896 | UAGCAGAGUACU | 618 | 318-340_C21A |
| | | UACUCUGCUA | | | | GUCCACAGCCA | | |
| AD-61245 UM | A-122879 | CUGUGGACAGU | 619 | 321-341 | A-122880 | UGAGCAGAGUAC | 620 | 319-341 |
| | | ACUCUGCUCA | | | | UGUCCACAGCC | | |
| AD-61268 UM | A-122871 | UGUGGACAGUA | 621 | 322-342 | A-122872 | UUGAGCAGAGUA | 622 | 320-342 |
| | | CUCUGCUCAA | | | | CUGUCCACAGC | | |
| AD-61251 UM | A-122881 | GUGGACAGUAC | 623 | 323-343 | A-122882 | UUUGAGCAGAGU | 624 | 321-343 |
| | | UCUGCUCAAA | | | | ACUGUCCACAG | | |
| AD-61281 UM | A-122891 | UGGACAGUAC | 625 | 324-344 | A-122892 | CUUUGAGCAGAG | 626 | 322-344 |
| | | UCUGCUCAAAG | | | | UACUGUCCACA | | |
| AD-61258 UM | A-122899 | UGGACAGUACU | 627 | 324-344_G21U | A-122900 | AUUUGAGCAGAG | 628 | 322-344_G21U |
| | | CUGCUCAAAU | | | | UACUGUCCACA | | |
| AD-61275 UM | A-122889 | GGACAGUACU | 629 | 325-345 | A-122890 | UCUUUGAGCAGA | 630 | 323-345 |
| | | CUGCUCAAAGA | | | | GUACUGUCCAC | | |
| AD-61269 UM | A-122887 | GACAGUACUCU | 631 | 326-346 | A-122888 | UUCUUUGAGCAG | 632 | 324-346 |
| | | GCUCAAAGAA | | | | AGUACUGUCCA | | |
| AD-61279 UM | A-122955 | UGUGGGCCAGG | 633 | 423-443 | A-122956 | UAAGGUUUCUCC | 634 | 421-443 |
| | | AGAAACCUUA | | | | UGGCCCACAAU | | |
| AD-61278 UM | A-122937 | CCAGGAGAAAC | 635 | 429-449 | A-122938 | UUUUGAUAAGGU | 636 | 427-449 |
| | | CUUAUCAAAA | | | | UUCUCCUGGCC | | |
| AD-61247 UM | A-122911 | UGUUCUACAUU | 637 | 506-526 | A-122912 | UAAUUGGCUUAA | 638 | 504-526 |
| | | AAGCCAAUUA | | | | UGUAGAACAUU | | |
| AD-61241 UM | A-122909 | GUUCUACAUUA | 639 | 507-527 | A-122910 | UUAAUUGGCUUA | 640 | 505-527 |
| | | AGCCAAUUAA | | | | AUGUAGAACAU | | |
| AD-61282 UM | A-122907 | UUCUACAUUAA | 641 | 508-528 | A-122908 | CUUAAUUGGCUU | 642 | 506-528 |
| | | GCCAAUUAAG | | | | AAUGUAGAACA | | |
| AD-61265 UM | A-122917 | UUCUACAUUAA | 643 | 508-528_G21A | A-122918 | UUUAAUUGGCUU | 644 | 506-528_G21A |
| | | GCCAAUUAAA | | | | AAUGUAGAACA | | |
| AD-61276 UM | A-122905 | UCUACAUUAAG | 645 | 509-529 | A-122906 | ACUUAAUUGGC | 646 | 507-529 |
| | | CCAAUUAAGU | | | | UUAAUGUAGAAC | | |
| AD-61250 UM | A-122865 | CUACAUUAAGC | 647 | 510-530 | A-122866 | UACUUAAUUGGC | 648 | 508-530 |
| | | CAAUUAAGUA | | | | UUAAUGUAGAA | | |
| AD-61256 UM | A-122867 | UACAUUAAGCC | 649 | 511-531 | A-122868 | AUACUUAAUUGG | 650 | 509-531 |
| | | AAUUAAGUAU | | | | CUUAAUGUAGA | | |
| AD-61262 UM | A-122869 | ACAUUAAGCCA | 651 | 512-532 | A-122870 | UAUACUUAAUUG | 652 | 510-532 |
| | | AUUAAGUAUA | | | | GCUUAAUGUAG | | |
| AD-61244 UM | A-122863 | CAUUAAGCCAA | 653 | 513-533 | A-122864 | UUAUACUUAAUU | 654 | 511-533 |
| | | UUAAGUAUAA | | | | GGCUUAAUGUA | | |
| AD-61238 UM | A-122861 | AUUAAGCCAAU | 655 | 514-534 | A-122862 | UUUAUACUUAAU | 656 | 512-534 |
| | | UAAGUAUAAA | | | | UGGCUUAAUGU | | |
| AD-61280 UM | A-122875 | UUAAGCCAAUU | 657 | 515-535 | A-122876 | CUUUAUACUUAA | 658 | 513-535 |
| | | AAGUAUAAAG | | | | UUGGCUUAAUG | | |
| AD-61240 UM | A-122893 | UUAAGCCAAUU | 659 | 515-535_G21U | A-122894 | AUUUAUACUUAA | 660 | 513-535_G21U |
| | | AAGUAUAAAU | | | | UUGGCUUAAUG | | |
| AD-61263 UM | A-122885 | UAAGCCAAUUA | 661 | 516-536 | A-122886 | CCUUUAUACUUA | 662 | 514-536 |
| | | AGUAUAAAGG | | | | AUUGGCUUAAU | | |
| AD-61252 UM | A-122897 | UAAGCCAAUUA | 663 | 516-536_G21A | A-122898 | UCUUUAUACUUA | 664 | 514-536_G21A |
| | | AGUAUAAAGA | | | | AUUGGCUUAAU | | |
| AD-61274 UM | A-122873 | AAGCCAAUUAA | 665 | 517-537 | A-122874 | ACCUUUAUACUU | 666 | 515-537 |
| | | GUAUAAAGGU | | | | AAUUGGCUUAA | | |
| AD-61270 UM | A-122903 | AGCCAAUUAAG | 667 | 518-538 | A-122904 | GACCUUUAUACU | 668 | 516-538 |
| | | UAUAAAGGUC | | | | UAAUUGGCUUA | | |
| AD-61264 UM | A-122901 | AGCCAAUUAAG | 669 | 518-538_C21A | A-122902 | UACCUUUAUACU | 670 | 516-538_C21A |
| | | UAUAAAGGUA | | | | UAAUUGGCUUA | | |
| AD-61255 UM | A-122945 | GCCCUUGCAGA | 671 | 573-593 | A-122946 | GGAUAAACUUUC | 672 | 571-593 |
| | | AAGUUUAUCC | | | | UGCAAGGGCAA | | |
| AD-61283 UM | A-122923 | GCCCUUGCAGA | 673 | 573-593_C21A | A-122924 | UGAUAAACUUUC | 674 | 571-593_C21A |
| | | AAGUUUAUCA | | | | UGCAAGGGCAA | | |
| AD-61284 UM | A-122939 | CCCUUGCAGAA | 675 | 574-594 | A-122940 | AGGAUAAACUUU | 676 | 572-594 |
| | | AGUUUAUCCU | | | | CUGCAAGGGCA | | |
| AD-61267 UM | A-122950 | CCAAUGGUCAG | 677 | 666-686 | A-122952 | UUUUGAAGAUCU | 678 | 664-686 |
| | | AUCUUCAAAA | | | | GACCAUUGGCC | | |
| AD-61266 UM | A-122933 | CAAUGGUCAGA | 679 | 667-687 | A-122934 | AUUUUGAAGAUC | 680 | 665-687 |
| | | UCUUCAAAAU | | | | UGACCAUUGGC | | |
| AD-61273 UM | A-122953 | AAUGGUCAGAU | 681 | 668-688 | A-122954 | UAUUUUGAAGAU | 682 | 666-688 |
| | | CUUCAAAAUA | | | | CUGACCAUUGG | | |
| AD-61272 UM | A-122935 | UGGUCAGAUCU | 683 | 670-690 | A-122936 | UUUAUUUUGAAG | 684 | 668-690 |
| | | UCAAAAUAAA | | | | AUCUGACCAUU | | |

The iRNA molecules described herein can include naturally occurring nucleotides or can include at least one modified nucleotide, including, but not limited to a 2′-O-methyl modified nucleotide, a nucleotide having a 5′-phosphorothioate group, and a terminal nucleotide linked to a cholesteryl derivative. Alternatively, the modified nucleotide may be chosen from the group of: a 2′-deoxy-2′-fluoro modified nucleotide, a 2′-deoxy-modified nucleotide, a locked nucleotide, an acyclic nucleotide, an abasic nucleotide, 2′-amino-modified nucleotide, 2′-alkyl-modified nucleotide, morpholino nucleotide, a phosphoramidate, and a non-natural base comprising nucleotide. Such a modified sequence can be based, e.g., on a first sequence of said iRNA selected from the group consisting of the sense sequences of Tables 2-3, 5-6 and 9-10 of WO2015/050990, and a second sequence selected from the group consisting of the corresponding antisense sequences of Tables 2-3, 5-6 and 9-10 of WO2015/050990.

In one embodiment, the iRNA targets a wildtype LECT2 RNA transcript variant. In another embodiment, the iRNA targets a mutant transcript (e.g., a LECT2 RNA carrying an allelic variant). For example, an iRNA featured in the invention can target a polymorphic variant, such as a single nucleotide polymorphism (SNP), of LECT2.

In some embodiments, the iRNA (e.g., dsRNA) targets (e.g., reduces) mRNA that encodes valine at position 40 in the mature LECT2 protein (or amino acid 58 in the unprocessed protein). In some embodiments, the iRNA (e.g., dsRNA) targets (e.g., reduces) mRNA that encodes isoleucine at position 40 in the mature LECT2 protein (or amino acid 58 in the unprocessed protein). In another embodiment, the iRNA (e.g., dsRNA) targets (e.g., reduces) both mRNA that encodes valine and mRNA that encodes isoleucine at position 40 in the mature LECT2 protein (or amino acid 58 in the unprocessed protein).

In another embodiment, the iRNA targets both a wildtype and a mutant LECT2 transcript. In yet another embodiment, the iRNA targets a particular transcript variant of LECT2. In yet another embodiment, the iRNA agent targets multiple transcript variants.

In one embodiment, an iRNA featured in the invention targets a non-coding region of a LECT2 RNA transcript, such as the 5′ or 3′ untranslated region of a transcript.

In some embodiments, an iRNA as described herein is in the form of a conjugate, e.g., a carbohydrate conjugate, which may serve as a targeting moiety and/or ligand, as described herein. In one embodiment, the conjugate is attached to the 3′ end of the sense strand of the dsRNA. In some embodiments, the conjugate is attached via a linker, e.g., via a bivalent or trivalent branched linker.

In some embodiments, the conjugate comprises one or more N-acetylgalactosamine (GalNAc) derivatives. Such a conjugate is also referred to herein as a GalNAc conjugate. In some embodiments, the conjugate targets the iRNA (e.g., dsRNA) to a particular cell, e.g., a liver cell, e.g., a hepatocyte. The GalNAc derivatives can be attached via a linker, e.g., a bivalent or trivalent branched linker. In particular embodiments, the conjugate is

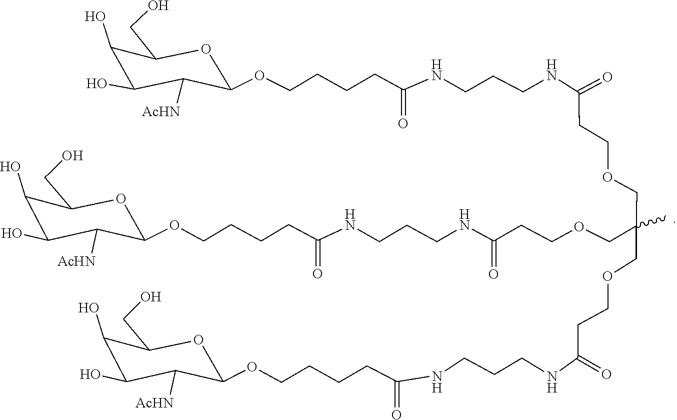 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In some embodiments, the iRNA is attached to the carbohydrate conjugate via a linker, e.g., a linker as shown in the following schematic, wherein X is O or S

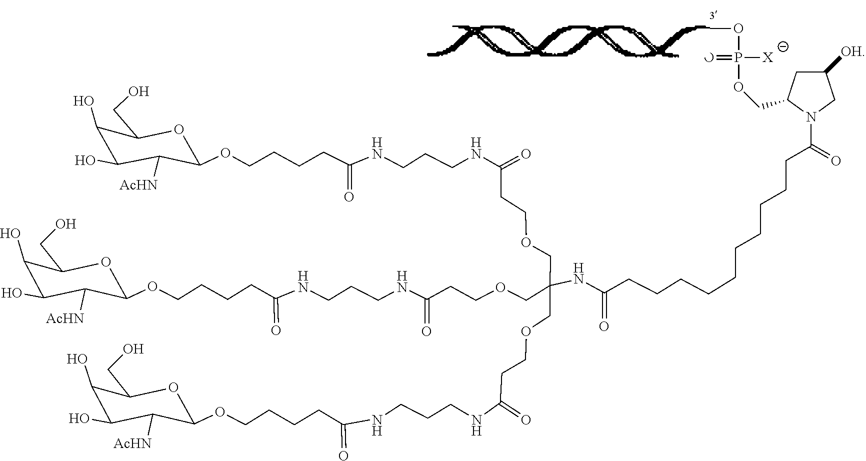

In some embodiments, X is O. In some embodiments, X is S.

In some embodiments, the iRNA is conjugated to L96 as defined in Table 1 and shown below

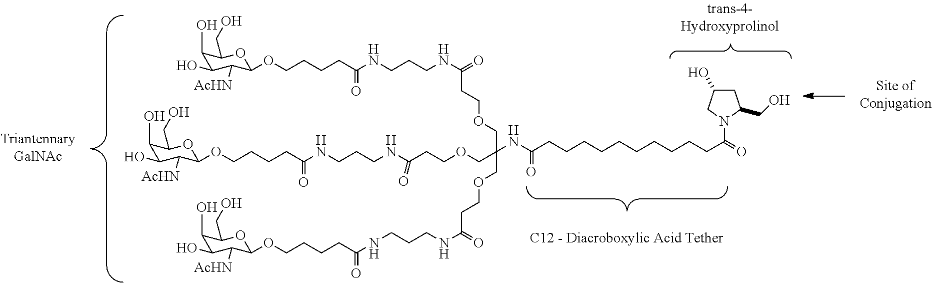

In some embodiments, the iRNA is conjugated to a ligand that targets the iRNA (e.g., dsRNA) to a desired organ (e.g., the liver) or to a particular cell type (e.g., hepatocytes). In some embodiments, the iRNA is conjugated to a ligand (e.g., a GalNAc ligand, e.g., L96) that targets the iRNA (e.g., dsRNA) to the liver.

In one embodiment, the iRNA (e.g., dsRNA) is provided in a pharmaceutical composition for inhibiting the expression of a LECT2 gene in an organism, generally a human subject. The composition typically includes one or more of the iRNAs described herein and a pharmaceutically acceptable carrier or delivery vehicle. In one embodiment, the composition is used for treating a disorder related to LECT2 expression, e.g., amyloidosis, e.g., LECT2 amyloidosis.

In one embodiment, an iRNA provided herein is a dsRNA for inhibiting expression of LECT2, wherein said dsRNA comprises a sense strand and an antisense strand 15-30 base pairs in length and the antisense strand is complementary to at least 15 contiguous nucleotides of SEQ ID NO: 1.

In one embodiment, an iRNA provided herein is a dsRNA comprising a sense strand complementary to an antisense strand, wherein said antisense strand comprises a region of complementarity to a LECT2 RNA transcript, wherein each strand has about 14 to about 30 nucleotides, wherein said double stranded RNAi agent is represented by formula (III):

(III)
sense:
5′nₚ-Nₐ-(X X X)ᵢ-N_{b}-Y Y Y-N_{b}-(Z Z Z)ⱼ-Nₐ-n_{q}3′
antisense:
3′nₚ′-Nₐ′-(X′X′X′)ₖ-N_{b}′-Y′Y′Y′-N_{b}′-(Z′Z′Z′)ₗ-Nₐ′-n_{q}′5′

wherein:
i, j, k, and 1 are each independently 0 or 1;
p, p′, q, and q′ are each independently 0-6;
each Nₐ and Nₐ′ independently represents an oligonucleotide sequence comprising 0-25 nucleotides which are either modified or unmodified or combinations thereof, each sequence comprising at least two differently modified nucleotides;
each N_{b} and N_{b}′ independently represents an oligonucleotide sequence comprising 0-10 nucleotides which are either modified or unmodified or combinations thereof;
each nₚ, nₚ′, n_{q}, and n_{q}′ independently represents an overhang nucleotide;
XXX, YYY, ZZZ, X′X′X′, Y′Y′Y′, and Z′Z′Z′ each independently represent one motif of three identical modifications on three consecutive nucleotides;
modifications on N_{b} differ from the modification on Y and modifications on N_{b}′ differ from the modification on Y′.

In some embodiments, the sense strand is conjugated to at least one ligand.

In some embodiments, i is 1; j is 1; or both i and j are 1. In some embodiments, k is 1; 1 is 1; or both k and 1 are 1. In some embodiments, XXX is complementary to X′X′X′, YYY is complementary to Y′Y′Y′, and ZZZ is complementary to Z′Z′Z′. In some embodiments, the Y′Y′Y′ motif occurs at the 11, 12 and 13 positions of the antisense strand from the 5′-end. In some embodiments, the Y′ is 2′-O-methyl.

In some embodiments, the duplex region is 15-30 nucleotide pairs in length. In some embodiments, the duplex region is 17-23 nucleotide pairs in length. In some embodiments, the duplex region is 19-21 nucleotide pairs in length. In some embodiments, the duplex region is 21-23 nucleotide pairs in length.

In some embodiments, the modifications on the nucleotides are selected from the group consisting of a locked nucleic acid (LNA), an acyclic nucleotide, a hexitol or hexose nucleic acid (HNA), a cyclohexene nucleic acid (CeNA), 2′-methoxyethyl, 2′-O-alkyl, 2′-O-allyl, 2′-C-allyl, 2′-fluoro, 2′-deoxy, 2′-hydroxyl, and any combination thereof. In some embodiments, the modifications on the nucleotides are 2′-O-methyl, 2′-fluoro or both.

In some embodiments, the ligand comprises a carbohydrate. In some embodiments, the ligand is attached via a linker. In some embodiments, the linker is a bivalent or trivalent branched linker.

In some embodiments, the ligand is

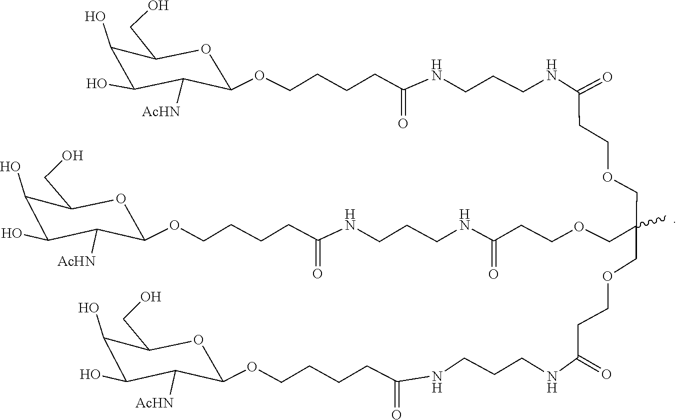 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In some embodiments, the ligand and linker are as shown in Formula XXIV:

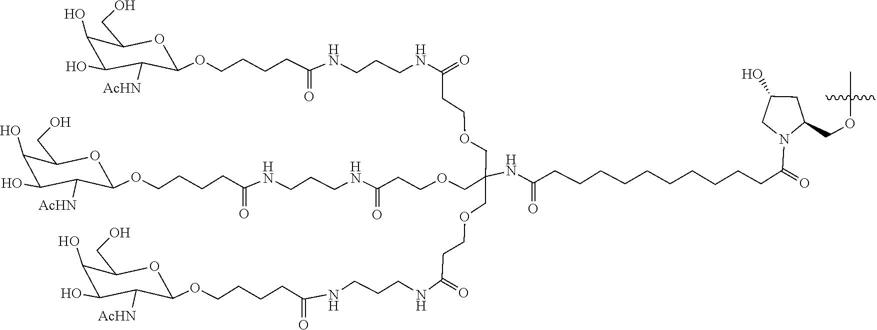 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

In some embodiments, the ligand is attached to the 3′ end of the sense strand.

In some embodiments, the dsRNA has (e.g., comprises) a nucleotide sequence (e.g., a sense and/or antisense sequence) selected from the group of sequences provided in Tables 2-3, 5-6 and 9-10 of WO2015/050990.

In some embodiments, an iRNA provided herein is a dsRNA for inhibiting expression of LECT2, wherein said dsRNA comprises a sense strand and an antisense strand, the antisense strand comprising a region of complementarity to a LECT2 RNA transcript, which antisense strand comprises at least 15 contiguous nucleotides differing by no more than 3 nucleotides from one of the antisense sequences listed in any one of Tables 2-3, 5-6 and 9-10 of WO2015/050990.

In some embodiments, the dsRNA comprises at least one modified nucleotide. In some embodiments, at least one of the modified nucleotides is chosen from the group consisting of: a 2′-O-methyl modified nucleotide, a nucleotide comprising a 5′-phosphorothioate group, and a terminal nucleotide linked to a cholesteryl derivative or dodecanoic acid bisdecylamide group. In some embodiments, the modified nucleotide is chosen from the group consisting of: a 2′-deoxy-2′-fluoro modified nucleotide, a 2′-deoxy-modified nucleotide, a locked nucleotide, an acyclic nucleotide, an abasic nucleotide, 2′-amino-modified nucleotide, 2′-alkyl-modified nucleotide, morpholino nucleotide, a phosphoramidate, and a non-natural base comprising nucleotide.

In some embodiments, the region of complementarity is at least 17 nucleotides in length. In some embodiments, the region of complementarity is between 19 and 21 nucleotides in length. In some embodiments, the region of complementarity is 19 nucleotides in length. In some embodiments, each strand is no more than 30 nucleotides in length.

In some embodiments, at least one strand comprises a 3′ overhang of at least 1 nucleotide. In some embodiments, at least one strand comprises a 3′ overhang of at least 2 nucleotides.

In some embodiments, an iRNA (e.g., a dsRNA) described herein further comprises a ligand. In some embodiments, the ligand is a GalNAc ligand. In some embodiments, the ligand targets the iRNA (e.g., the dsRNA) to the liver (e.g., to hepatocytes). In some embodiments, the ligand is conjugated to the 3′ end of the sense strand of the dsRNA.

In some embodiments, the region of complementarity consists of an antisense sequence selected from the antisense sequences provided in Tables 2-3, 5-6 and 9-10 of WO2015/050990.

In some embodiments, the region of complementarity consists of an antisense sequence selected from a duplex disclosed herein, wherein the duplex suppresses LECT2 mRNA or protein expression by at least 20%, 30%, 40%, 50%, 60%, 70%, 80%, 85% or 90%.

In some embodiments, the dsRNA comprises a sense strand comprising or consisting of a sense strand sequence selected from Table 2, 3, 5, 6, 9 or 10 of WO2015/050990, and an antisense strand comprising or consisting of an antisense sequence selected from Table 2, 3, 5, 6, 9 or 10 of WO2015/050990. In some embodiments, the dsRNA comprises or consists of a pair of corresponding sense and antisense sequences selected from those of the duplexes disclosed in Tables 2-3 and 5-11 of WO2015/050990. In certain embodiments, the dsRNA comprises or consists of a pair of corresponding sense and antisense sequences selected from those of the duplexes disclosed in Table 8 of WO2015/050990.

In some embodiments, the iRNA (e.g., dsRNA) is provided in a pharmaceutical composition for inhibiting expression of a LECT2 gene.

In some embodiments, the iRNA (e.g., dsRNA) is administered in an unbuffered solution. In some embodiments, the unbuffered solution is saline or water. In some embodiments, the iRNA (e.g., dsRNA) is administered with a buffer solution. In some embodiments, the buffer solution comprises acetate, citrate, prolamine, carbonate, or phosphate or any combination thereof. In some embodiments, the buffer solution is phosphate buffered saline (PBS).

In some embodiments, the iRNA (e.g., dsRNA) is targeted to the liver (e.g., to hepatocytes). In some embodiments, the iRNA (e.g., dsRNA) is administered intravenously. In some embodiments, the iRNA (e.g., dsRNA) is administered subcutaneously.

In some embodiments, the iRNA (e.g., dsRNA) comprises a ligand (e.g., a GalNAc ligand) that targets the iRNA (e.g., dsRNA) to a liver cell, e.g., a hepatocyte.

In some embodiments, a pharmaceutical composition comprises an iRNA (e.g., a dsRNA) described herein that comprises a ligand (e.g., a GalNAc ligand), and the pharmaceutical composition is administered subcutaneously. In some embodiments, the ligand targets the iRNA (e.g., dsRNA) to a liver cell, e.g., a hepatocyte.

In certain embodiments, the pharmaceutical composition includes a lipid formulation. In some embodiments, the iRNA is in a LNP formulation, e.g., a MC3 formulation. In some embodiments, the LNP formulation targets the iRNA to a particular cell, e.g., a liver cell (e.g., a hepatocyte). In some embodiments, the lipid formulation is a LNP11 formulation. In some embodiments, the composition is administered intravenously.

In another embodiment, the iRNA (e.g., dsRNA) is formulated for administration according to a dosage regimen described herein, e.g., not more than once every four weeks, not more than once every three weeks, not more than once every two weeks, or not more than once every week. In another embodiment, the administration of the pharmaceutical composition can be maintained for a month or longer, e.g., one, two, three, or six months, or one year or longer.

In another embodiment, a composition containing an iRNA described herein, e.g., a dsRNA targeting LECT2, is administered in conjunction with a second therapy for a disorder related to LECT2 expression (e.g., a LECT2 amyloidosis). An iRNA or composition comprising an iRNA provided herein can be administered before, after, or concurrent with a second therapy.

In some embodiments, the iRNA is administered before the second therapy. In some embodiments, the iRNA is administered after the second therapy. In some embodiments, the iRNA is administered concurrent with the second therapy.

In some embodiments, the second therapy is a non-iRNA therapeutic agent that is effective to treat the disorder or symptoms of the disorder.

In some embodiments, the disorder to be treated by the compositions or methods disclosed herein is a LECT2 amyloidosis that affects kidney function, e.g., through amyloid deposition in the kidney. In some such embodiments, the iRNA is administered in conjunction with a therapy that supports kidney function (e.g., dialysis). In some embodiments, the iRNA is administered in conjunction with a diuretic, an ACE (angiotensin converting enzyme) inhibitor, an angiotensin receptor blocker, and/or dialysis, e.g., to support or manage kidney function.

In some embodiments, the disorder to be treated by the compositions or methods disclosed herein is a LECT2 amyloidosis involving amyloid deposits in the liver. In some such embodiments, the iRNA is administered in conjunction with a therapy that supports liver function.

In some embodiments, the disorder to be treated by the compositions or methods disclosed herein is a LECT2 amyloidosis, and the iRNA is administered in conjunction with removal of all or part of the organ(s) affected by the amyloidosis (e.g., resection of all or part of kidney or liver tissue affected by the amyloidosis). The removal is optionally conducted in conjunction with a replacement of all or part of the organ removed (e.g., in conjunction with a kidney or liver organ transplant).

In some embodiments, the iRNA (e.g., dsRNA) is used in a method of inhibiting LECT2 expression in a cell, the method comprising: (a) introducing into the cell the iRNA (e.g., dsRNA) described herein and (b) maintaining the cell of step (a) for a time sufficient to obtain degradation of the mRNA transcript of a LECT2 gene, thereby inhibiting expression of the LECT2 gene in the cell.

In some embodiments, the iRNA (e.g., dsRNA) is used in a method for reducing or inhibiting the expression of a LECT2 gene in a cell (e.g., a liver cell, e.g., a hepatocyte). The method includes contacting the cell with a dsRNA as described herein, thereby inhibiting expression of a LECT2 gene. “Contacting,” as used herein, includes directly contacting a cell, as well as indirectly contacting a cell. For example, a cell within a subject (e.g., a liver cell) may be contacted when a composition comprising an RNAi is administered (e.g., intravenously or subcutaneously) to the subject.

In some embodiments, the method includes:

(a) introducing into the cell a dsRNA, wherein the dsRNA includes at least two sequences that are complementary to each other. The dsRNA has a sense strand having a first sequence and an antisense strand having a second sequence; the antisense strand has a region of complementarity that is substantially complementary to at least a part of an mRNA encoding LECT2, and where the region of complementarity is 30 nucleotides or less, e.g., 15-30 nucleotides in length, and generally 19-24 nucleotides in length, and where the dsRNA upon contact with a cell expressing LECT2, inhibits expression of a LECT2 gene by at least 10%, e.g., at least 20%, at least 30%, at least 40% or more; and

(b) maintaining the cell of step (a) for a time sufficient to obtain degradation of the mRNA transcript of the LECT2 gene, thereby reducing or inhibiting expression of a LECT2 gene in the cell.

In some embodiments, the cell is treated ex vivo, in vitro, or in vivo. In some embodiments, the cell is a hepatocyte. In some embodiments, the cell is present in a subject in need of treatment, prevention and/or management of a disorder related to LECT2 expression. In some embodiments, the disorder is a LECT2 amyloidosis, as described herein. In some embodiments, the expression of LECT2 is inhibited by at least 30%.

In some embodiments, the iRNA (e.g., dsRNA) has an IC₅₀ in the range of 0.0005-1 nM, e.g., between 0.001 and 0.2 nM, between 0.002 and 0.1 nM, between 0.005 and 0.075 nM, or between 0.01 and 0.05 nM. In some embodiments, the iRNA (e.g., dsRNA) has an IC₅₀ equal to or less than 0.02 nM, e.g., between 0.0005 and 0.02 nM, between 0.001 and 0.02 nM, between 0.005 and 0.02 nM, or between 0.01 and 0.02 nM. In some embodiments, the iRNA (e.g., dsRNA) has an IC₅₀ in the range of 0.01-1 nM.

In some embodiments, the cell (e.g., the hepatocyte) is a mammalian cell (e.g., a human, non-human primate, or rodent cell). In one embodiment, the subject is a mammal (e.g., a human) having a LECT2 amyloidosis. In one embodiment, the dsRNA introduced reduces or inhibits expression of a LECT2 gene in the cell.

In one embodiment, the dsRNA inhibits expression of a LECT2 gene, or inhibits amyloid deposition (e.g., by preventing amyloid deposition or reducing amyloid deposition, e.g., by reducing size, number, or extent of amyloid deposits). The inhibition optionally involves an inhibition of at least 5%, 10%, 15%, 20%, 25%, 30%, 35%, 40%, 45%, 50% or more compared to a reference, (e.g., a control that is untreated or treated with a non-targeting dsRNA (e.g., a dsRNA that does not target LECT2)).

In other embodiments, the iRNA (e.g., dsRNA) is used in a method for treating pathological processes related to LECT2 expression (e.g., amyloid deposition). In one embodiment, the method includes administering to a subject, e.g., a patient in need of such treatment, an effective (e.g., a therapeutically or prophylactically effective) amount of an iRNA (e.g., dsRNA) provided herein.

In one embodiment, the iRNA (e.g., dsRNA) is used in a method of treating and/or preventing a disorder related to LECT2 expression (e.g., a LECT2 amyloidosis) comprising administering to a subject in need of such treatment a therapeutically effective amount of an iRNA (e.g., a dsRNA) described herein, or a composition comprising an iRNA (e.g., a dsRNA) described herein.

In another embodiment, the iRNA (e.g., dsRNA) is used in a method of treating a disorder related to LECT2 expression (e.g., LECT2 amyloidosis) comprising administering to a subject in need of such treatment an iRNA (e.g., dsRNA), wherein said iRNA (e.g., dsRNA) comprises a sense strand and an antisense strand 15-30 base pairs in length and the antisense strand is complementary to at least 15 contiguous nucleotides of a LECT2 mRNA transcript, e.g., a human LECT2 mRNA transcript, e.g., SEQ ID NO: 1 or a nucleotide sequence having an A to G substitution at nucleotide position 373 of SEQ ID NO: 1. In one embodiment, the iRNA (e.g., dsRNA) targets mRNA that encodes valine at position 40 in the mature LECT2 protein (or amino acid 58 in the unprocessed protein).

In one embodiment, the iRNA (e.g., dsRNA) is used in a method of treating a subject having a LECT2 amyloidosis, the method comprising administering to the subject an iRNA (e.g., a dsRNA), wherein said iRNA (e.g., dsRNA) comprises a sense strand and an antisense strand 15-30 base pairs in length and the antisense strand is complementary to at least 15 contiguous nucleotides of a LECT2 mRNA transcript, e.g., a human LECT2 mRNA transcript, e.g., SEQ ID NO: 1 or a nucleotide sequence having an A to G substitution at nucleotide position 373 of SEQ ID NO: 1. In one embodiment, the iRNA (e.g., dsRNA) targets mRNA that encodes valine at position 40 in the mature LECT2 protein (or amino acid 58 in the unprocessed protein).

In some embodiments, administration of the iRNA targeting LECT2 alleviates or relieves the severity of at least one symptom of a disorder related to LECT2 expression in the patient. In one embodiment, subject has a LECT2 amyloidosis. In another embodiment, the subject is at risk for developing a LECT2 amyloidosis.

In some embodiments, the iRNA (e.g., dsRNA) is formulated as an LNP formulation. In some embodiments, the iRNA (e.g., dsRNA) is in the form of a GalNAc conjugate.

In some embodiments, the iRNA (e.g., dsRNA) is administered at a dose of 0.05-50 mg/kg. In some embodiments, the iRNA (e.g., dsRNA) is administered at a concentration of 0.01 mg/kg-5 mg/kg bodyweight of the subject.

In some embodiments, the iRNA (e.g., dsRNA) is formulated as an LNP formulation and is administered at a dose of 0.05-5 mg/kg. In some embodiments, the iRNA (e.g., dsRNA) is formulated as an LNP formulation and is administered at a dose of 0.1 to 0.5 mg/kg. In some embodiments, the iRNA (e.g., dsRNA) is in the form of a GalNAc conjugate and is administered at a dose of 0.5-50 mg/kg. In some embodiments, the iRNA (e.g., dsRNA) is in the form of a GalNAc conjugate and is administered at a dose of 1 to 10 mg/kg.

In some embodiments, the method inhibits expression of a LECT2 gene, or inhibits amyloid deposition (e.g., by preventing amyloid deposition or reducing amyloid deposition, e.g., by reducing size, number, or extent of amyloid deposits). The inhibition optionally involves an inhibition of at least 5%, 10%, 15%, 20%, 25%, 30%, 35%, 40%, 45%, 50%, 55%, 60%, 65%, 70%, 75%, 80%, 85%, or 90% compared to a reference (e.g., a control that is untreated or treated with a non-targeting dsRNA (e.g., a dsRNA that does not target LECT2)).

In some embodiments, the iRNA (e.g., dsRNA) has an IC₅₀ in the range of 0.0005-1 nM, e.g., between 0.001 and 0.2 nM, between 0.002 and 0.1 nM, between 0.005 and 0.075 nM, or between 0.01 and 0.05 nM. In some embodiments, the iRNA (e.g., dsRNA) has an IC₅₀ equal to or less than 0.02 nM, e.g., between 0.0005 and 0.02 nM, between 0.001 and 0.02 nM, between 0.005 and 0.02 nM, or between 0.01 and 0.02 nM. In some embodiments, the iRNA (e.g., dsRNA) has an IC₅₀ in the range of 0.01-1 nM.

In some embodiments, the method ameliorates a symptom associated with a LECT2 related disorder (e.g., a LECT2 amyloidosis). In some embodiments, the method inhibits expression of a LECT2 gene in the subject. In some embodiments, the method inhibits amyloid deposition (e.g., by preventing amyloid deposition or reducing amyloid deposition, e.g., by reducing size, number, or extent of amyloid deposits).

In some embodiments, the iRNA (e.g., dsRNA) or composition comprising the iRNA is administered according to a dosing regimen.

In some embodiments, the subject is of Mexican descent (e.g., a Mexican American). In some embodiments, the subject carries the G allele of the LECT2 gene that encodes valine at position 40 in the mature protein (amino acid 58 in the unprocessed protein). In some embodiments, the subject is homozygous for the G allele (G/G genotype). In some embodiments, a LECT2 protein expressed in the subject has valine at position 40 in the mature protein (or at amino acid 58 in the unprocessed protein).

In some embodiments, the iRNA (e.g., dsRNA) or composition comprising the iRNA is administered repeatedly, e.g., according to a dosing regimen. In some embodiments, the iRNA (e.g., dsRNA) or composition comprising the iRNA is administered subcutaneously. In some embodiments, the iRNA is in the form of a GalNAc conjugate. In some embodiments, the iRNA (e.g., the dsRNA) is administered at a dose of 0.5-50 mg/kg. In some embodiments, the iRNA (e.g., dsRNA) is in the form of a GalNAc conjugate and is administered at a dose of 1 to 10 mg/kg.

Other exemplary iRNAs (e.g., dsRNAs), including, but not limited to, modifications and conjugates, are described in International Application Publication No. WO 2015/050990, the content of which is incorporated by reference in its entirety.

### IV. Antisense Polynucleotide Agents

The nucleic acid agents described herein include, e.g., antisense polynucleotide agents. The antisense polynucleotide agents described herein can target nucleic acids encoding a LECT2 gene and interfere with the normal function of the targeted nucleic acid. The LECT2 nucleic acid may be within a cell, e.g., a cell within a subject, such as a human. The antisense polynucleotide agents described herein can be used to treat a subject having a disorder that would benefit from inhibiting or reducing the expression of a LECT2 mRNA, e.g., a LECT2-associated disease, such as amyloidosis, e.g., a LECT2 amyloidosis (ALECT2).

In one embodiment, the antisense polynucleotide agent inhibits expression of a LECT2 gene. The agents comprise about 4 to about 50 contiguous nucleotides, wherein the nucleotide sequence of the agent is about 80% complementary over its entire length to the equivalent region of the nucleotide sequence of any one of SEQ ID NOs: 1-4 of International Application Publication No. WO2016/164746.

In one embodiment, the equivalent region is one of the target regions of SEQ ID NO: 1 provided in Table 3, e.g., SEQ ID NOs: 109-204, of WO2016/164746.

In one embodiment, the nucleotides of an antisense polynucleotide agent described herein are un-modified, and do not comprise, e.g., chemical modifications and/or conjugations known in the art and described herein (e.g., as shown in Table 4). In another embodiment, one or more (e.g., at least 5, 10, 11, 12, 13, 14, 15, 16, 17, 18, 19, 20 or more) of the nucleotides of an antisense polynucleotide agent of the invention is chemically modified (e.g., as shown in Table 4).

In one embodiment, the antisense polynucleotide agent comprises at least 8 contiguous nucleotides differing by no more than 3 nucleotides from any one of the nucleotide sequences listed in Table 4.

TABLE 4
Antisense polynucleotides targeting LECT2

| | SEQ | | | SEQ | mRNA | SEQ | Unmodified |
|---|---|---|---|---|---|---|---|
| | ID | Modified Antisense | | ID | Target | ID | Antisense |
| Oligo | NO: | Sequence (5′ to 3′) | Pos | NO: | Sequence | NO: | Sequence |
| A-133875.1 | 13 | ususcsusasdAsdTsdAsdTsdTs(5MdC) | 12 | 109 | CUAUCCAUGG | 205 | UUCUAAUAUUCCAUGGAUAG |
| | | s(5MdC)sdAsdTsdGsgsasusasg | | | AAUAUUAGAA | | |
| A-133876.1 | 14 | gsasgscsasdAsdGsdTs(5MdC)sdAsdAsdG | 24 | 110 | UAUUAGAACU | 206 | GAGCAAGUCAAGUUCUAAUA |
| | | sdTsdTs(5MdC)susasasusa | | | UGACUUGCUC | | |
| A-133877.1 | 15 | asasgsasgsdGsdAsdTsdGsdGsdAsdGs | 33 | 111 | UUGACUUGCU | 207 | AAGAGGAUGGAGCAAGUCAA |
| | | (5MdC)sdAsdAsgsuscsasa | | | CCAUCCUCUU | | |
| A-133878.1 | 16 | ascsasasasdAsdAsdGsdTsdTsdT | 44 | 112 | CAUCCUCUUA | 208 | ACAAAAAGUUUAAGAGGAUG |
| | | sdAsdAsdGsdAsgsgsasusg | | | AACUUUUUGU | | |
| A-133879.1 | 17 | usususasgsdTsdGsdTsdGsdAsdGsdAs | 57 | 113 | UUUUUGUGUC | 209 | UUUAGUGUGAGACACAAAAA |
| | | (5MdC)sdAs(5MdC)sasasasasa | | | UCACACUAAA | | |
| A-133880.1 | 18 | uscsuscsasdTsdTsdTs(5MdC) | 66 | 114 | CUCACACUAA | 210 | UCUCAUUUCUUUAGUGUGAG |
| | | sdTsdTsdTsdAsdGsdTsgsusgsasg | | | AGAAAUGAGA | | |
| A-133881.1 | 19 | gsasasusus(5MdC)sdTsdGs(5MdC)sdAs | 78 | 115 | AAAUGAGAGA | 211 | GAAUUCUGCAUCUCUCAUUU |
| | | dTs(5MdC)sdTs(5MdC)sdTscsasususu | | | UGCAGAAUUC | | |
| A-133882.1 | 20 | usasusususdAsdGs(5MdC)s(5MdC) | 90 | 116 | CAGAAUUCUA | 212 | UAUUUAGCCUUAGAAUUCUG |
| | | sdTsdTsdAsdGsdAsdAsususcsusg | | | AGGCUAAAUA | | |
| A-133883.1 | 21 | ascsususcs(5MdC)sdTsdAsdGs(5MdC) | 100 | 117 | AGGCUAAAUA | 213 | ACUUCCUAGCUAUUUAGCCU |
| | | sdTsdAsdTsdTsdTsasgscscsu | | | GCUAGGAAGU | | |
| A-133884.1 | 22 | gsusususgsdAsdAsdTsdGsdAsdAsdT | 112 | 118 | UAGGAAGUAU | 214 | GUUUGAAUGAAUACUUCCUA |
| | | sdAs(5MdC)sdTsuscscsusa | | | UCAUUCAAAC | | |
| A-133885.1 | 23 | asasusasusdTs(5MdC)sdAsdAsdGsd | 121 | 119 | UUCAUUCAAA | 215 | AAUAUUCAAGUUUGAAUGAA |
| | | TsdTsdTsdGsdAsasusgsasa | | | CUUGAAUAUU | | |
| A-133886.1 | 24 | uscsuscsusdTsdTsdGsdAsdAsdGsd | 132 | 120 | UUGAAUAUUC | 216 | UCUCUUUGAAGAAUAUUCAA |
| | | AsdAsdTsdAsususcsasa | | | UUCAAAGAGA | | |
| A-133887.1 | 25 | gsususgscs(5MdC)s(5MdC)s(5MdC)s | 145 | 121 | AAAGAGAGUG | 217 | GUUGCCCCCACACUCUCUUU |
| | | (5MdC)sdAs(5MdC)sdAs(5MdC) | | | UGGGGGCAAC | | |
| | | sdTs(5MdC)suscsususu | | | | | |
| A-133888.1 | 26 | csusgsasusdTsdAsdGsdAsdGsdTs | 154 | 122 | GUGGGGGCAA | 218 | CUGAUUAGAGUUGCCCCCAC |
| | | dTsdGs(5MdC)s(5MdC)scscscsasc | | | CUCUAAUCAG | | |
| A-133889.1 | 27 | usasgsususdTs(5MdC)sdTsdTs(5MdC)s | 166 | 123 | CUAAUCAGAG | 219 | UAGUUUCUUCCUCUGAUUAG |
| | | (5MdC)sdTs(5MdC)sdTsdGsasususasg | | | GAAGAAACUA | | |
| A-133890.1 | 28 | usususascsdTsdTs(5MdC)s(5MdC) | 177 | 124 | AAGAAACUAA | 220 | UUUACUUCCUUUAGUUUCUU |
| | | sdTsdTsdTsdAsdGsdTsususcsusu | | | AGGAAGUAAA | | |
| A-133891.1 | 29 | asascsasus(5MdC)sdTsdGsdGsdTsd | 187 | 125 | AGGAAGUAAA | 221 | AACAUCUGGUUUUACUUCCU |
| | | TsdTsdTsdAs(5MdC)sususcscsu | | | ACCAGAUGUU | | |
| A-133892.1 | 30 | gsgscsususdTsdGsdGsdTsdGsdGsd | 200 | 126 | AGAUGUUUUC | 222 | GGCUUUGGUGGAAAACAUCU |
| | | AsdAsdAsdAscsasuscsu | | | CACCAAAGCC | | |
| A-133893.1 | 31 | gscscsasasdAsdAsdGsdGsdAsdGsd | 211 | 127 | ACCAAAGCCC | 223 | GCCAAAAGGAGGGCUUUGGU |
| | | GsdGs(5MdC)sdTsususgsgsu | | | UCCUUUUGGC | | |
| A-133894.1 | 32 | asasuscsasdGsdAs(5MdC)s(5MdC) | 221 | 128 | UCCUUUUGGC | 224 | AAUCAGACCAGCCAAAAGGA |
| | | sdAsdGs(5MdC)s(5MdC) | | | UGGUCUGAUU | | |
| | | sdAsdAsasasgsgsa | | | | | |
| A-133895.1 | 33 | gsusgscsgsdGsdTsdAsdGsdAsd | 231 | 129 | UGGUCUGAUU | 225 | GUGCGGUAGAAAUCAGACCA |
| | | AsdAsdTs(5MdC)sdAsgsascscsa | | | UCUACCGCAC | | |
| A-133896.1 | 34 | asusgsgscs(5MdC)s(5MdC)sdTsdGs | 243 | 130 | UACCGCACUG | 226 | AUGGCCCUGCCAGUGCGGUA |
| | | (5MdC)s(5MdC)sdAsdGsdTsdGs | | | GCAGGGCCAU | | |
| | | csgsgsusa | | | | | |
| A-133897.1 | 35 | usasusasusdTsdAsdGs(5MdC)s(5MdC)s | 254 | 131 | CAGGGCCAUG | 227 | UAUAUUAGCCCAUGGCCCUG |
| | | (5MdC)sdAsdTsdGsdGscscscsusg | | | GGCUAAUAUA | | |
| A-133898.1 | 36 | csususgscs(5MdC)sdAsdGs(5MdC)sdAs | 266 | 132 | CUAAUAUAUG | 228 | CUUGCCAGCACAUAUAUUAG |
| | | (5MdC)sdAsdTsdAsdTsasususasg | | | UGCUGGCAAG | | |
| A-133899.1 | 37 | csasususgsdGsdAsdAsdGsdAs(5MdC) | 276 | 133 | UGCUGGCAAG | 229 | CAUUGGAAGACUUGCCAGCA |
| | | sdTsdTsdGs(5MdC)scsasgscsa | | | UCUUCCAAUG | | |
| A-133900.1 | 38 | ascsgsuscs(5MdC)sdGsdGsdAsdTs | 288 | 134 | UUCCAAUGAG | 230 | ACGUCCGGAUCUCAUUGGAA |
| | | (5MdC)sdTs(5MdC)sdAsdTsusgsgsasa | | | AUCCGGACGU | | |
| A-133901.1 | 39 | asusgsgscsdGsdGsdTs(5MdC)sdAs | 299 | 135 | UCCGGACGUG | 231 | AUGGCGGUCACACGUCCGGA |
| | | (5MdC)sdAs(5MdC)sdGsdTscscsgsgsa | | | UGACCGCCAU | | |
| A-133902.1 | 40 | gsuscscsas(5MdC)sdAsdGs(5MdC) | 309 | 136 | UGACCGCCAU | 232 | GUCCACAGCCAUGGCGGUCA |
| | | s(5MdC)sdAsdTsdGsdGs(5MdC) | | | GGCUGUGGAC | | |
| | | sgsgsuscsa | | | | | |
| A-133903.1 | 41 | gsasgscsasdGsdAsdGsdTsdAs(5MdC) | 321 | 137 | CUGUGGACAG | 233 | GAGCAGAGUACUGUCCACAG |
| | | sdTsdGsdTs(5MdC)scsascsasg | | | UACUCUGCUC | | |
| A-133904.1 | 42 | usgsascsusdTs(5MdC)sdTsdTsdTs | 331 | 138 | UACUCUGCUC | 234 | UGACUUCUUUGAGCAGAGUA |
| | | dGsdAsdGs(5MdC)sdAsgsasgsusa | | | AAAGAAGUCA | | |
| A-133905.1 | 43 | gsusgsasgsdGs(5MdC)s(5MdC)sdTs | 341 | 139 | AAAGAAGUCA | 235 | GUGAGGCCUCUGACUUCUUU |
| | | (5MdC)sdTsdGsdAs(5MdC) | | | GAGGCCUCAC | | |
| | | sdTsuscsususu | | | | | |
| A-133906.1 | 44 | uscscsascsdAs(5MdC)s(5MdC)s(5MdC) | 352 | 140 | AGGCCUCACC | 236 | UCCACACCCUGGUGAGGCCU |
| | | sdTsdGsdGsdTsdGsdAsgsgscscsu | | | AGGGUGUGGA | | |
| A-133907.1 | 45 | gsasgscsas(5MdC)sdAsdAsdGsdAsdT | 364 | 141 | GGUGUGGACA | 237 | GAGCACAAGAUGUCCACACC |
| | | sdGsdTs(5MdC)s(5MdC)sascsascsc | | | UCUUGUGCUC | | |
| A-133908.1 | 46 | asgsasuscs(5MdC)sdAsdGs(5MdC)sdAs | 374 | 142 | UCUUGUGCUC | 238 | AGAUCCAGCAGAGCACAAGA |
| | | dGsdAsdGs(5MdC)sdAscsasasgsa | | | UGCUGGAUCU | | |
| A-133909.1 | 47 | usgscsgsusdAs(5MdC)sdAs(5MdC) | 386 | 143 | CUGGAUCUAC | 239 | UGCGUACACAGUAGAUCCAG |
| | | sdAsdGsdTsdAsdGsdAsuscscsasg | | | UGUGUACGCA | | |
| A-133910.1 | 48 | csasgsusgsdAsdAsdTsdGsdGsdTsdGs | 396 | 144 | UGUGUACGCA | 240 | CAGUGAAUGGUGCGUACACA |
| | | (5MdC)sdGsdTsascsascsa | | | CCAUUCACUG | | |
| A-133911.1 | 49 | csascsasasdTs(5MdC)sdAsdTsdTs | 407 | 145 | CAUUCACUGG | 241 | CACAAUCAUUCCAGUGAAUG |
| | | (5MdC)s(5MdC)sdAsdGs | | | AAUGAUUGUG | | |
| | | dTsgsasasusg | | | | | |
| A-133912.1 | 50 | ususcsuscs(5MdC)sdTsdGsdGs(5MdC) | 418 | 146 | AUGAUUGUGG | 242 | UUCUCCUGGCCCACAAUCAU |
| | | s(5MdC)s(5MdC)sdAs(5MdC) | | | GCCAGGAGAA | | |
| | | sdAsasuscsasu | | | | | |
| A-133913.1 | 51 | gsususususdGsdAsdTsdAsdAsd | 431 | 147 | AGGAGAAACC | 243 | GUUUUGAUAAGGUUUCUCCU |
| | | GsdGsdTsdTsdTscsuscscsu | | | UUAUCAAAAC | | |
| A-133914.1 | 52 | usasgscsasdTsdTs(5MdC)sdTsdT | 441 | 148 | UUAUCAAAAC | 244 | UAGCAUUCUUGUUUUGAUAA |
| | | sdGsdTsdTsdTsdTsgsasusasa | | | AAGAAUGCUA | | |
| A-133915.1 | 53 | cscsasususdAsdTsdTsdGsdAsd | 451 | 149 | AAGAAUGCUA | 245 | CCAUUAUUGAUAGCAUUCUU |
| | | TsdAsdGs(5MdC)sdAsususcsusu | | | UCAAUAAUGG | | |
| A-133916.1 | 54 | gsasusasusdTs(5MdC)sdGsdAsdAs | 463 | 150 | AAUAAUGGUG | 246 | GAUAUUCGAACACCAUUAUU |
| | | (5MdC)sdAs(5MdC)s(5MdC) | | | UUCGAAUAUC | | |
| | | sdAsususasusu | | | | | |
| A-133917.1 | 55 | asasascscsdTs(5MdC)sdTsdTs(5MdC) | 475 | 151 | CGAAUAUCUG | 247 | AAACCUCUUCCAGAUAUUCG |
| | | s(5MdC)sdAsdGsdAsdTsasususcsg | | | GAAGAGGUUU | | |
| A-133918.1 | 56 | ususususgsdAs(5MdC)sdAs(5MdC)sdAs | 486 | 152 | AAGAGGUUUU | 248 | UUUUGACACAAAAACCUCUU |
| | | dAsdAsdAsdAs(5MdC)scsuscsusu | | | UGUGUCAAAA | | |
| A-133919.1 | 57 | usgsusasgsdAsdAs(5MdC)sdAsdTsdTs | 495 | 153 | UUGUGUCAAA | 249 | UGUAGAACAUUUUGACACAA |
| | | dTsdTsdGsdAscsascsasa | | | AUGUUCUACA | | |
| A-133920.1 | 58 | ususasasusdTsdGsdGs(5MdC) | 508 | 154 | UUCUACAUUA | 250 | UUAAUUGGCUUAAUGUAGAA |
| | | sdTsdTsdAsdAsdTsdGsusasgsasa | | | AGCCAAUUAA | | |
| A-133921.1 | 59 | cscsusususdAsdTsdAs(5MdC)sdTsd | 517 | 155 | AAGCCAAUUA | 251 | CCUUUAUACUUAAUUGGCUU |
| | | TsdAsdAsdTsdTsgsgscsusu | | | AGUAUAAAGG | | |
| A-133922.1 | 60 | ususcsususdAsdAsdTsdAsdGsdGsdAs | 529 | 156 | UAUAAAGGUC | 252 | UUCUUAAUAGGACCUUUAUA |
| | | (5MdC)s(5MdC)sdTsususasusa | | | CUAUUAAGAA | | |
| A-133923.1 | 61 | asgsusususdTsdTs(5MdC)sdTs(5MdC) | 541 | 157 | AUUAAGAAGG | 253 | AGUUUUUCUCCCUUCUUAAU |
| | | s(5MdC)s(5MdC)sdTsdTs(5MdC) | | | GAGAAAAACU | | |
| | | sususasasu | | | | | |
| A-133924.1 | 62 | asusasgsasdGsdTsdTs(5MdC)s(5MdC) | 552 | 158 | AGAAAAACUU | 254 | AUAGAGUUCCAAGUUUUUCU |
| | | sdAsdAsdGsdTsdTsusususcsu | | | GGAACUCUAU | | |
| A-133925.1 | 63 | gscsasasgsdGsdGs(5MdC)sdAsdA | 561 | 159 | UGGAACUCUA | 255 | GCAAGGGCAAUAGAGUUCCA |
| | | sdTsdAsdGsdAsdGsususcscsa | | | UUGCCCUUGC | | |
| A-133926.1 | 64 | asusasasas(5MdC)sdTsdTsdTs(5MdC) | 572 | 160 | UGCCCUUGCA | 256 | AUAAACUUUCUGCAAGGGCA |
| | | sdTsdGs(5MdC)sdAsdAsgsgsgscsa | | | GAAAGUUUAU | | |
| A-133927.1 | 65 | asususgsusdAsdTsdGs(5MdC)s | 585 | 161 | AGUUUAUCCU | 257 | AUUGUAUGCCAGGAUAAACU |
| | | (5MdC)sdAsdGsdGsdAsdTsasasascsu | | | GGCAUACAAU | | |
| A-133928.1 | 66 | gscsascsasdTsdGs(5MdC) | 594 | 162 | UGGCAUACAA | 258 | GCACAUGCGAUUGUAUGCCA |
| | | sdGsdAsdTsdTsdGsdTsdAsusgscscsa | | | UCGCAUGUGC | | |
| A-133929.1 | 67 | asgsusususdTs(5MdC)sdAsdAsdTsdG | 606 | 163 | GCAUGUGCAC | 259 | AGUUUUCAAUGUGCACAUGC |
| | | sdTsdGs(5MdC)sdAscsasusgsc | | | AUUGAAAACU | | |
| A-133930.1 | 68 | ascsuscsgsdAsdGsdTs(5MdC)sdAs | 617 | 164 | UUGAAAACUG | 260 | ACUCGAGUCACAGUUUUCAA |
| | | (5MdC)sdAsdGsdTsdTsususcsasa | | | UGACUCGAGU | | |
| A-133931.1 | 69 | csasgsusasdGsdGsdGsdTs(5MdC)sdAs | 627 | 165 | UGACUCGAGU | 261 | CAGUAGGGUCACUCGAGUCA |
| | | (5MdC)sdTs(5MdC)sdGsasgsuscsa | | | GACCCUACUG | | |
| A-133932.1 | 70 | usususascsdAsdGsdGsdTsdAsdTsdGs | 639 | 166 | CCCUACUGCA | 262 | UUUACAGGUAUGCAGUAGGG |
| | | (5MdC)sdAsdGsusasgsgsg | | | UACCUGUAAA | | |
| A-133933.1 | 71 | usgsgscscsdTsdTs(5MdC)sdGsdAsdT | 649 | 167 | UACCUGUAAA | 263 | UGGCCUUCGAUUUACAGGUA |
| | | sdTsdTsdAs(5MdC)sasgsgsusa | | | UCGAAGGCCA | | |
| A-133934.1 | 72 | asasgsasus(5MdC)sdTsdGsdAs(5MdC) | 662 | 168 | AAGGCCAAUG | 264 | AAGAUCUGACCAUUGGCCUU |
| | | s(5MdC)sdAsdTsdTsdGsgscscsusu | | | GUCAGAUCUU | | |
| A-133935.1 | 73 | usususususdAsdTsdTsdTsdTsdGsd | 673 | 169 | UCAGAUCUUC | 265 | UUUUUAUUUUGAAGAUCUGA |
| | | AsdAsdGsdAsuscsusgsa | | | AAAAUAAAAA | | |
| A-133936.1 | 74 | ususasasgsdAsdTsdGsdAs(5MdC) | 683 | 170 | AAAAUAAAAA | 266 | UUAAGAUGACUUUUUAUUUU |
| | | sdTsdTsdTsdTsdTsasusususu | | | GUCAUCUUAA | | |
| A-133937.1 | 75 | csasuscscsdAsdGsdGsdTsdTsd | 694 | 171 | UCAUCUUAAA | 267 | CAUCCAGGUUUUUAAGAUGA |
| | | TsdTsdTsdAsdAsgsasusgsa | | | AACCUGGAUG | | |
| A-133938.1 | 76 | gsasasgsgsdGsdTsdAsdTsdGs(5MdC) | 704 | 172 | AACCUGGAUG | 268 | GAAGGGUAUGCAUCCAGGUU |
| | | sdAsdTs(5MdC)s(5MdC)sasgsgsusu | | | CAUACCCUUC | | |
| A-133939.1 | 77 | asasususus(5MdC)sdTsdTsdGsdAsd | 717 | 173 | ACCCUUCUCU | 269 | AAUUUCUUGAAGAGAAGGGU |
| | | AsdGsdAsdGsdAsasgsgsgsu | | | UCAAGAAAUU | | |
| A-133940.1 | 78 | gsusgsasas(5MdC)sdAs(5MdC)sdAsd | 726 | 174 | UUCAAGAAAU | 270 | GUGAACACAAAUUUCUUGAA |
| | | AsdAsdTsdTsdTs(5MdC)sususgsasa | | | UUGUGUUCAC | | |
| A-133941.1 | 79 | csasusususdTsdTs(5MdC)s(5MdC) | 738 | 175 | GUGUUCACAA | 271 | CAUUUUUCCUUUGUGAACAC |
| | | sdTsdTsdTsdGsdTsdGsasascsasc | | | AGGAAAAAUG | | |
| A-133942.1 | 80 | csasuscscs(5MdC)sdTsdTs(5MdC) | 750 | 176 | GAAAAAUGCA | 272 | CAUCCCUUCAUGCAUUUUUC |
| | | sdAsdTsdGs(5MdC)sdAsdTsususususc | | | UGAAGGGAUG | | |
| A-133943.1 | 81 | asusgsgsgsdGsdTsdAsdTs(5MdC)s(5MdC) | 760 | 177 | UGAAGGGAUG | 273 | AUGGGGUAUCCAUCCCUUCA |
| | | sdAsdTs(5MdC)s(5MdC)scsususcsa | | | GAUACCCCAU | | |
| A-133944.1 | 82 | asusgsuscsdAsdTsdGsdGsdAsdAsd | 772 | 178 | UACCCCAUUU | 274 | AUGUCAUGGAAAAUGGGGUA |
| | | AsdAsdTsdGsgsgsgsusa | | | UCCAUGACAU | | |
| A-133945.1 | 83 | gsusasasusdAsdAsdTs(5MdC)sdAsdT | 781 | 179 | UUCCAUGACA | 275 | GUAAUAAUCAUGUCAUGGAA |
| | | sdGsdTs(5MdC)sdAsusgsgsasa | | | UGAUUAUUAC | | |
| A-133946.1 | 84 | asgsgscsasdTsdGs(5MdC) | 794 | 180 | UUAUUACACA | 276 | AGGCAUGCAAUGUGUAAUAA |
| | | sdAsdAsdTsdGsdTsdGsdTsasasusasa | | | UUGCAUGCCU | | |
| A-133947.1 | 85 | gsususususdGsdAsdTsdAs(5MdC) | 804 | 181 | UUGCAUGCCU | 277 | GUUUUGAUACAGGCAUGCAA |
| | | sdAsdGsdGs(5MdC)sdAsusgscsasa | | | GUAUCAAAAC | | |
| A-133948.1 | 86 | gsusascsgsdTsdGsdAsdGsdAsd | 815 | 182 | UAUCAAAACA | 278 | GUACGUGAGAUGUUUUGAUA |
| | | TsdGsdTsdTsdTsusgsasusa | | | UCUCACGUAC | | |
| A-133949.1 | 87 | usasusgsusdTsdTsdAsdTsdGsd | 827 | 183 | UCACGUACCU | 279 | UAUGUUUAUGAGGUACGUGA |
| | | AsdGsdGsdTsdAscsgsusgsa | | | CAUAAACAUA | | |
| A-133950.1 | 88 | csasusasgsdGsdTsdGsdTsdAs | 837 | 184 | CAUAAACAUA | 280 | CAUAGGUGUAUAUGUUUAUG |
| | | dTsdAsdTsdGsdTsususasusg | | | UACACCUAUG | | |
| A-133951.1 | 89 | usususgsusdGsdGsdGsdTsdAs | 847 | 185 | UACACCUAUG | 281 | UUUGUGGGUACAUAGGUGUA |
| | | (5MdC)sdAsdTsdAsdGsgsusgsusa | | | UACCCACAAA | | |
| A-133952.1 | 90 | asususasasdAsdAsdAsdAsdTsdTs | 858 | 186 | ACCCACAAAA | 282 | AUUAAAAAAUUUUUGUGGGU |
| | | dTsdTsdTsdGsusgsgsgsu | | | AUUUUUUAAU | | |
| A-133953.1 | 91 | ususcscsusdTsdTsdTsdTsdTsdT | 870 | 187 | UUUUUAAUUA | 283 | UUCCUUUUUUUAAUUAAAAA |
| | | sdAsdAsdTsdTsasasasasa | | | AAAAAAGGAA | | |
| A-133954.1 | 92 | asasascsus(5MdC)sdAsdAsdAsdTsdTsdTs | 880 | 188 | AAAAAAGGAA | 284 | AAACUCAAAUUUCCUUUUUU |
| | | (5MdC)s(5MdC)sdTsususususu | | | AUUUGAGUUU | | |
| A-133955.1 | 93 | usgsususus(5MdC)sdTsdAsdTsdTsdTsd | 891 | 189 | UUUGAGUUUA | 285 | UGUUUCUAUUUAAACUCAAA |
| | | AsdAsdAs(5MdC)suscsasasa | | | AAUAGAAACA | | |
| A-133956.1 | 94 | csususgscsdAsdTsdTsdTsdAsdTs | 904 | 190 | AGAAACAUGA | 286 | CUUGCAUUUAUCAUGUUUCU |
| | | (5MdC)sdAsdTsdGsusususcsu | | | UAAAUGCAAG | | |
| A-133957.1 | 95 | asusgsususdTsdTs(5MdC)sdTsdTsdTs | 915 | 191 | AAAUGCAAGA | 287 | AUGUUUUCUUUCUUGCAUUU |
| | | (5MdC)sdTsdTsdGscsasususu | | | AAGAAAACAU | | |
| A-133958.1 | 96 | asasasasus(5MdC)sdAsdAsdAsdAsd | 924 | 192 | AAAGAAAACA | 288 | AAAAUCAAAAUGUUUUCUUU |
| | | TsdGsdTsdTsdTsuscsususu | | | UUUUGAUUUU | | |
| A-133959.1 | 97 | gsascsasasdTsdGsdAsdGsdTsdTs | 935 | 193 | UUUGAUUUUA | 289 | GACAAUGAGUUAAAAUCAAA |
| | | dAsdAsdAsdAsuscsasasa | | | ACUCAUUGUC | | |
| A-133960.1 | 98 | gsasascsasdTs(5MdC)sdAsdGsdAsd | 947 | 194 | UCAUUGUCAC | 290 | GAACAUCAGAGUGACAAUGA |
| | | GsdTsdGsdAs(5MdC)sasasusgsa | | | UCUGAUGUUC | | |
| A-133961.1 | 99 | csasgsusus(5MdC)sdAs(5MdC)sdAsdTsd | 957 | 195 | UCUGAUGUUC | 291 | CAGUUCACAUGAACAUCAGA |
| | | GsdAsdAs(5MdC)sdAsuscsasgsa | | | AUGUGAACUG | | |
| A-133962.1 | 100 | asgscscscsdGsdAsdAsdGs(5MdC) | 970 | 196 | UGAACUGGUU | 292 | AGCCCGAAGCAACCAGUUCA |
| | | sdAsdAs(5MdC)s(5MdC)sdAsgsususcsa | | | GCUUCGGGCU | | |
| A-133963.1 | 101 | csasgsasus(5MdC)sdAsdAsdAsdGsdAs | 980 | 197 | GCUUCGGGCU | 293 | CAGAUCAAAGAGCCCGAAGC |
| | | dGs(5MdC)s(5MdC)s(5MdC)sgsasasgsc | | | CUUUGAUCUG | | |
| A-133964.1 | 102 | uscscsasusdAsdGsdGsdTsdGsdAs | 991 | 198 | UUUGAUCUGU | 294 | UCCAUAGGUGACAGAUCAAA |
| | | (5MdC)sdAsdGsdAsuscsasasa | | | CACCUAUGGA | | |
| A-133965.1 | 103 | ascscsascsdTs(5MdC)sdAsdGsdAsd | 1002 | 199 | ACCUAUGGAA | 295 | ACCACUCAGAUUCCAUAGGU |
| | | TsdTs(5MdC)s(5MdC)sdAsusasgsgsu | | | UCUGAGUGGU | | |
| A-133966.1 | 104 | asasasasasdAsdTsdAsdAsdAsdAs(5MdC) | 1012 | 200 | UCUGAGUGGU | 296 | AAAAAAUAAAACCACUCAGA |
| | | s(5MdC)sdAs(5MdC)suscsasgsa | | | UUUAUUUUUU | | |
| A-133967.1 | 105 | csusgsasgsdAsdAsdAsdTs(5MdC) | 1023 | 201 | UUAUUUUUUA | 297 | CUGAGAAAUCUAAAAAAUAA |
| | | sdTsdAsdAsdAsdAsasasusasa | | | GAUUUCUCAG | | |
| A-133968.1 | 106 | usasgsasus(5MdC)sdTsdTsdTsdG | 1036 | 202 | UUCUCAGUCC | 298 | UAGAUCUUUGGGACUGAGAA |
| | | sdGsdGsdAs(5MdC)sdTsgsasgsasa | | | CAAAGAUCUA | | |
| A-133969.1 | 107 | ususasususdTsdAsdTs(5MdC) | 1046 | 203 | CAAAGAUCUA | 299 | UUAUUUAUCUUAGAUCUUUG |
| | | sdTsdTsdAsdGsdAsdTscsusususg | | | AGAUAAAUAA | | |
| A-133970.1 | 108 | gsususcsus(5MdC)sdTsdTsdGsd | 1056 | 204 | AGAUAAAUAA | 300 | GUUCUCUUGUUUAUUUAUCU |
| | | TsdTsdTsdAsdTsdTsusasuscsu | | | ACAAGAGAAC | | |

In some embodiments, substantially all of the nucleotides of the antisense polynucleotide agent are modified nucleotides. In other embodiment, all of the nucleotides of the antisense polynucleotide agent are modified nucleotides.

The antisense polynucleotide agent may be 10 to 40 nucleotides in length; 10 to 30 nucleotides in length; 18 to 30 nucleotides in length; 10 to 24 nucleotides in length; 18 to 24 nucleotides in length; 14-20 nucleotides in length; or 14 or 20 nucleotides in length.

In one embodiment, the modified nucleotide comprises a modified sugar moiety selected from the group consisting of: a 2′-O-methoxyethyl modified sugar moiety, a 2′-methoxy modified sugar moiety, a 2′-O-alkyl modified sugar moiety, and a bicyclic sugar moiety.

In one embodiment, the bicyclic sugar moiety has a (—CH2-)n group forming a bridge between the 2′ oxygen and the 4′ carbon atoms of the sugar ring, wherein n is 1 or 2.

In another embodiment, the modified nucleotide is a 5-methylcytosine. In one embodiment, the modified nucleotide comprises a modified internucleoside linkage, such as a phosphorothioate internucleoside linkage.

In one embodiment, the antisense polynucleotide agent comprises a plurality of 2′-deoxynucleotides flanked on each side by at least one nucleotide having a modified sugar moiety.

In one embodiment, the antisense polynucleotide agent is a gapmer comprising a gap segment comprised of linked 2′-deoxynucleotides positioned between a 5′ and a 3′ wing segment.

In one embodiment, the modified sugar moiety is selected from the group consisting of a 2′-O-methoxyethyl modified sugar moiety, a 2′-methoxy modified sugar moiety, a 2′-O-alkyl modified sugar moiety, and a bicyclic sugar moiety.

In one embodiment, the 5′-wing segment is 1 to 6 nucleotides in length, e.g., 2, 3, 4, or 5 nucleotides in length. In one embodiment, the 3′-wing segment is 1 to 6 nucleotides in length, e.g., 2, 3, 4, or 5 nucleotides in length. In one embodiment, the gap segment is 5 to 14 nucleotides in length, e.g., 10 nucleotides in length.

In one aspect, the present invention provides antisense polynucleotide agent for inhibiting LECT2 gene expression, comprising a gap segment consisting of linked deoxynucleotides; a 5′-wing segment consisting of linked nucleotides; a 3′-wing segment consisting of linked nucleotides; wherein the gap segment is positioned between the 5′-wing segment and the 3′-wing segment and wherein each nucleotide of each wing segment comprises a modified sugar.

In one embodiment, the gap segment is ten 2′-deoxynucleotides in length and each of the wing segments is five nucleotides in length. In another embodiment, the gap segment is ten 2′-deoxynucleotides in length and each of the wing segments is four nucleotides in length. In yet another embodiment, the gap segment is ten 2′-deoxynucleotides in length and each of the wing segments is three nucleotides in length. In another embodiment, the gap segment is ten 2′-deoxynucleotides in length and each of the wing segments is two nucleotides in length.

In one embodiment, the modified sugar moiety is selected from the group consisting of a 2′-O-methoxyethyl modified sugar moiety, a 2′-methoxy modified sugar moiety, a 2′-O-alkyl modified sugar moiety, and a bicyclic sugar moiety.

In some embodiments, the antisense polynucleotide agent further comprises a ligand. In one embodiment, the antisense polynucleotide agent is conjugated to the ligand at the 3′-terminus.

In one embodiment, the ligand is an N-acetylgalactosamine (GalNAc) derivative.

In one embodiment, the ligand is

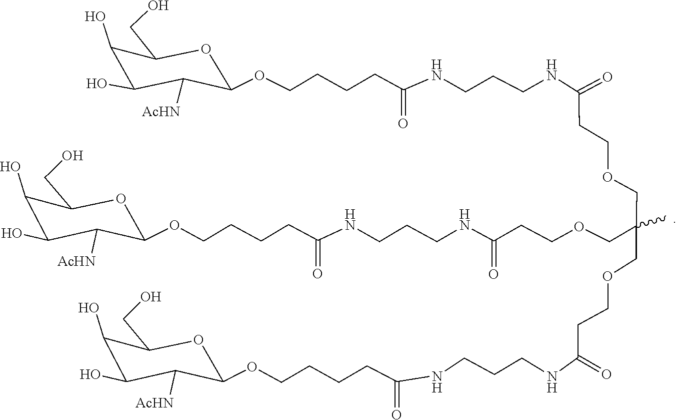 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In one embodiment, the antisense polynucleotide agent has a region that is substantially complementary to a portion of a LECT2 mRNA, e.g., a human LECT2 mRNA (e.g., a human LECT2 mRNA as provided in NM_002302.2 (SEQ ID NO: 1).

In some embodiments, the antisense polynucleotide agent has a region that is substantially complementary to a portion of a LECT2 mRNA transcript that has an A to G substitution at nucleotide position 373 of SEQ ID NO: 1. In some embodiments, the mRNA transcript encodes valine at position 40 in the mature LECT2 protein (or amino acid 58 in the unprocessed protein). In some embodiments, the mRNA transcript encodes isoleucine at position 40 in the mature LECT2 protein (or amino acid 58 in the unprocessed protein).

In one embodiment, an antisense polynucleotide agent as described herein targets a wildtype LECT2 RNA transcript variant, and in another embodiment, the antisense polynucleotide agent targets a mutant transcript (e.g., a LECT2 RNA carrying an allelic variant). For example, an antisense polynucleotide agent featured in the invention can target a polymorphic variant, such as a single nucleotide polymorphism (SNP), of LECT2.

In one embodiment, the antisense polynucleotide agent is present in a pharmaceutical composition for inhibiting expression of a LECT2 gene.

In one embodiment, the antisense polynucleotide agent is present in an unbuffered solution, such as saline or water. In another embodiment, the antisense polynucleotide agent is present in a buffer solution, such as a buffer comprising acetate, citrate, prolamine, carbonate, or phosphate or any combination thereof. In one embodiment, the buffer solution is phosphate buffered saline (PBS).

In one embodiment, the pharmaceutical composition comprises an antisense polynucleotide agent described herein and a lipid formulation, such as a lipid formulation comprising an LNP or a MC3.

In one embodiment, the antisense polynucleotide agent or pharmaceutical composition is used in a method of inhibiting LECT2 gene expression in a cell. The method includes contacting the cell with the agent or pharmaceutical composition; and maintaining the cell for a time sufficient to obtain antisense inhibition of a LECT2 gene, thereby inhibiting expression of the LECT2 gene in the cell.

In one embodiment, the cell is within a subject. In one embodiment, the subject is a human.

In one embodiment, the LECT2 gene expression is inhibited by at least about 30%, about 40%, about 50%, about 60%, about 70%, about 80%, about 90%, about 95%, about 98% or about 100%.

In another embodiment, the antisense polynucleotide agent or pharmaceutical composition is used in a method of treating a subject having a disease or disorder that would benefit from reduction in LECT2 gene expression. The method includes administering to the subject a therapeutically effective amount of the agent or pharmaceutical composition, thereby treating the subject.

In yet another embodiment, the antisense polynucleotide agent or pharmaceutical composition is used in a method of preventing at least one symptom in a subject having a disease or disorder that would benefit from reduction in LECT2 gene expression. The method includes administering to the subject a prophylactically effective amount of the agent or pharmaceutical composition, thereby preventing at least one symptom in the subject having a disorder that would benefit from reduction in LECT2 gene expression.

In one embodiment, the administration of the antisense polynucleotide agent to the subject causes a decrease in amyloid deposition (e.g., by preventing amyloid deposition or reducing amyloid deposition, e.g., by reducing size, number, or extent of amyloid deposits) or symptoms associated with amyloid deposition and/or a decrease in LECT2 protein levels.

In one embodiment, the administration of the antisense polynucleotide agent to the subject inhibits amyloid deposition (e.g., by preventing amyloid deposition or reducing amyloid deposition, e.g., by reducing size, number, or extent of amyloid deposits). The inhibition optionally involves an inhibition of at least 5%, 10%, 15%, 20%, 25%, 30%, 35%, 40%, 45%, 50% or more compared to a reference, (e.g., a control that is untreated or treated with a non-targeting dsRNA (e.g., a dsRNA that does not target LECT2)).

In one embodiment, the disorder is a LECT2-associated disease, e.g., a LECT2 amyloidosis. In some embodiments, the LECT2 amyloidosis is a renal amyloidosis. In some embodiments, the LECT2 amyloidosis involves amyloid deposition in the kidney. In some embodiments, LECT2 amyloidosis is associated with renal disease (e.g., renal insufficiency or nephrotic syndrome). In some embodiments, the amyloidosis is associated with proteinuria. In some embodiments, proteinuria is absent. In some embodiments, the LECT2 amyloidosis is a hepatic amyloidosis. In some embodiments, the LECT2 amyloidosis involves amyloid deposition in the liver. In some embodiments, the LECT2 amyloidosis is associated with inflammation in the liver (e.g., hepatitis, e.g., chronic hepatitis).

In one embodiment the subject is human. In some embodiments, the subject is of Mexican descent (e.g., a Mexican American).

In some embodiments, the subject carries the G allele of the LECT2 gene that encodes valine at position 40 in the mature protein (or amino acid 58 in the unprocessed protein). In some embodiments, the subject is homozygous for the G allele (G/G genotype). In some embodiments, a LECT2 protein expressed in the subject has valine at position 40 in the mature protein (or at amino acid 58 in the unprocessed protein).

In one embodiment, the antisense polynucleotide agent is administered at a dose of about 0.01 mg/kg to about 10 mg/kg or about 0.5 mg/kg to about 50 mg/kg. In one embodiment, the antisense polynucleotide agent is administered at a dose of about 10 to 100 mg/kg or 0.5 to 10 mg/kg.

In one embodiment, the antisense polynucleotide agent is administered to the subject once a week. In another embodiment, the antisense polynucleotide agent is administered to the subject twice a week. In yet another embodiment, the antisense polynucleotide agent is administered to the subject twice a month.

In one embodiment, the antisense polynucleotide agent is administered to the subject subcutaneously.

In another embodiment, the antisense polynucleotide agent is administered in conjunction with a second therapy for a disorder related to LECT2 expression (e.g., a LECT2 amyloidosis).

The antisense polynucleotide agent can be administered before, after, or concurrent with a second therapy. In some embodiments, the antisense polynucleotide agent is administered before the second therapy. In some embodiments, the antisense polynucleotide agent is administered after the second therapy. In some embodiments, the antisense polynucleotide agent is administered concurrent with the second therapy.

In some embodiments, the second therapy is a non-antisense polynucleotide therapeutic agent that is effective to treat the disorder or symptoms of the disorder.

In some embodiments, the disorder is a LECT2 amyloidosis that affects kidney function, e.g., through amyloid deposition in the kidney. In some such embodiments, the antisense polynucleotide agent is administered in conjunction with a therapy that supports kidney function (e.g., dialysis). In some embodiments, the antisense polynucleotide agent is administered in conjunction with a diuretic, an ACE (angiotensin converting enzyme) inhibitor, an angiotensin receptor blocker, and/or dialysis, e.g., to support or manage kidney function.

In some embodiments, the disorder is a LECT2 amyloidosis involving amyloid deposits in the liver. In some such embodiments, the antisense polynucleotide agent is administered in conjunction with a therapy that supports liver function.

In some embodiments, the disorder is a LECT2 amyloidosis, and the agent is administered in conjunction with removal of all or part of the organ(s) affected by the amyloidosis (e.g., resection of all or part of kidney or liver tissue affected by the amyloidosis). The removal is optionally conducted in conjunction with a replacement of all or part of the organ removed (e.g., in conjunction with a kidney or liver organ transplant).

In one embodiment, the method further includes administering an anti-LECT2 antibody, or antigen-binding fragment thereof, to the subject.

Other exemplary antisense polynucleotide agents, including, but not limited to, modifications and conjugates, are described in International Application Publication No. WO2016/164746, the content of which is incorporated by reference in its entirety.

### V. Modifications of iRNA and Antisense Polynucleotide Agents

The iRNAs (e.g., dsRNAs) and antisense polynucleotide agents described herein can be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position, or having an acyclic sugar) or replacement of the sugar, as well as (d) backbone modifications, including modification or replacement of the phosphodiester linkages. Specific examples of RNA compounds useful in this disclosure include, but are not limited to, RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In particular embodiments, the modified RNA will have a phosphorus atom in its internucleoside backbone.

Modified RNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

In other RNA mimetics suitable or contemplated for use in iRNAs and antisense polynucleotide agents, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA).

Some embodiments described herein include RNAs with phosphorothioate backbones and oligonucleosides with heteroatom backbones.

Modified RNAs may also contain one or more substituted sugar moieties. The iRNAs (e.g., dsRNAs) and antisense polynucleotide agents described herein can include one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl.

In other embodiments, an iRNA or antisense polynucleotide agent comprises one or more (e.g., about 1, 2, 3, 4, 5, 6, 7, 8, 9, 10, or more) acyclic nucleotides (or nucleosides). In certain embodiments, the sense strand or the antisense strand, or both sense strand and antisense strand, of an iRNA (e.g., dsRNA) include less than five acyclic nucleotides per strand (e.g., four, three, two or one acyclic nucleotides per strand). The one or more acyclic nucleotides can be found, for example, in the double-stranded region, of the sense or antisense strand, or both strands; at the 5′-end, the 3′-end, both of the 5′ and 3′-ends of the sense or antisense strand, or both strands, of the iRNA agent. In one embodiment, one or more acyclic nucleotides are present at positions 1 to 8 of the sense or antisense strand, or both. In one embodiment, one or more acyclic nucleotides are found in the antisense strand at positions 4 to 10 (e.g., positions 6-8) from the 5′-end of the antisense strand. In another embodiment, the one or more acyclic nucleotides are found at one or both 3′-terminal overhangs of the iRNA agent.

The term “acyclic nucleotide” or “acyclic nucleoside” as used herein refers to any nucleotide or nucleoside having an acyclic sugar, e.g., an acyclic ribose. An exemplary acyclic nucleotide or nucleoside can include a nucleobase, e.g., a naturally-occurring or a modified nucleobase (e.g., a nucleobase as described herein).

In certain embodiments, the acyclic nucleotide can be modified or derivatized, e.g., by coupling the acyclic nucleotide to another moiety, e.g., a ligand (e.g., a GalNAc, a cholesterol ligand), an alkyl, a polyamine, a sugar, a polypeptide, among others.

In other embodiments, the iRNA agent or antisense polynucleotide agent includes one or more acyclic nucleotides and one or more LNAs (e.g., an LNA as described herein).

An iRNA or antisense polynucleotide agent may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine.

The RNA of an iRNA or antisense polynucleotide agent can also be modified to include one or more (e.g., about 1, 2, 3, 4, 5, 6, 7, 8, 9, 10, or more) locked nucleic acids (LNA) (also referred to herein as “locked nucleotides”). In one embodiment, a locked nucleic acid is a nucleotide having a modified ribose moiety in which the ribose moiety comprises an extra bridge connecting, e.g., the 2′ and 4′ carbons.

In other embodiments, the iRNA or antisense polynucleotide agents include one or more (e.g., about 1, 2, 3, 4, 5, 6, 7, 8, 9, 10, or more) G-clamp nucleotides. A G-clamp nucleotide is a modified cytosine analog wherein the modifications confer the ability to hydrogen bond both Watson-Crick and Hoogsteen faces of a complementary guanine within a duplex.

Other exemplary modifications to iRNAs and antisense polynucleotide agents are described in International Application Publication No. WO2016/164746, the content of which is incorporated by reference in its entirety.

### VI. Conjugation of iRNA and Antisense Polynucleotide Agents

The iRNA and antisense polynucleotide agents disclosed herein can be in the form of conjugates. The conjugate may be attached at any suitable location in the iRNA molecule, e.g., at the 3′ end or the 5′ end of the sense or the antisense strand. The conjugates are optionally attached via a linker.

In some embodiments, an iRNA or antisense polynucleotide agent described herein is chemically linked to one or more ligands, moieties or conjugates, which may confer functionality, e.g., by affecting (e.g., enhancing) the activity, cellular distribution or cellular uptake of the iRNA. Such moieties include but are not limited to lipid moieties such as a cholesterol moiety, cholic acid, a thioether, e.g., beryl-S-tritylthiol, a thiocholesterol, an aliphatic chain, e.g., dodecandiol or undecyl residues, a phospholipid, e.g., di-hexadecyl-rac-glycerol or triethyl-ammonium 1,2-di-O-hexadecyl-rac-glycero-3-phosphonate, a polyamine or a polyethylene glycol chain, or adamantane acetic acid, a palmityl moiety, or an octadecylamine or hexylamino-carbonyloxycholesterol moiety.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin or hyaluronic acid); or a lipid. The ligand may also be a recombinant or synthetic molecule, such as a synthetic polymer, e.g., a synthetic polyamino acid. Ligands can also include targeting groups, e.g., a cell or tissue targeting agent, e.g., a lectin, glycoprotein, lipid or protein, e.g., an antibody, that binds to a specified cell type such as a liver cell.

In some embodiments, the ligand is a GalNAc ligand that comprises one or more N-acetylgalactosamine (GalNAc) derivatives. In some embodiments, the GalNAc ligand is used to target the iRNA to the liver (e.g., to hepatocytes).

Ligands can be proteins, e.g., glycoproteins, or peptides, e.g., molecules having a specific affinity for a co-ligand, or antibodies, e.g., an antibody, that binds to a specified cell type such as a cancer cell, endothelial cell, or bone cell, hormones and hormone receptors, non-peptidic species, such as lipids, lectins, carbohydrates, vitamins, cofactors, multivalent lactose, multivalent galactose, N-acetyl-galactosamine, N-acetyl-gulucosamine multivalent mannose, or multivalent fucose.

The ligand can be a substance, e.g., a drug, which can increase the uptake of the iRNA agent or antisense polynucleotide agents into the cell.

In one embodiment, the ligand is a lipid or lipid-based molecule. Such a lipid or lipid-based molecule can typically bind a serum protein, such as human serum albumin (HSA). In one embodiment, the lipid based ligand binds HSA. In another embodiment, the lipid based ligand binds HSA weakly or not at all, such that distribution of the conjugate to the kidney is enhanced. Other moieties that target to liver cells can also be used in place of or in addition to the lipid based ligand.

In another embodiment, the ligand is a moiety, e.g., a vitamin, which is taken up by a target cell, e.g., a proliferating cell.

In another embodiment, the ligand is a cell-permeation agent, such as a helical cell-permeation agent. In one embodiment, the agent is amphipathic.

The ligand can be a peptide or peptidomimetic. A “cell permeation peptide” is capable of permeating a cell, e.g., a microbial cell, such as a bacterial or fungal cell, or a mammalian cell, such as a human cell. In some embodiments, an iRNA oligonucleotide further comprises a carbohydrate. The carbohydrate conjugated iRNA are advantageous for the in vivo delivery of nucleic acids, as well as compositions suitable for in vivo therapeutic use, as described herein. As used herein, “carbohydrate” refers to a compound which is either a carbohydrate per se made up of one or more monosaccharide units having at least 6 carbon atoms (which can be linear, branched or cyclic) with an oxygen, nitrogen or sulfur atom bonded to each carbon atom; or a compound having as a part thereof a carbohydrate moiety made up of one or more monosaccharide units each having at least six carbon atoms (which can be linear, branched or cyclic), with an oxygen, nitrogen or sulfur atom bonded to each carbon atom. Representative carbohydrates include the sugars (mono-, di-, tri- and oligosaccharides containing from about 4, 5, 6, 7, 8, or 9 monosaccharide units), and polysaccharides such as starches, glycogen, cellulose and polysaccharide gums. Specific monosaccharides include C5 and above (e.g., C5, C6, C7, or C8) sugars; di- and trisaccharides include sugars having two or three monosaccharide units (e.g., C5, C6, C7, or C8).

In one embodiment, a carbohydrate conjugate comprises a monosaccharide. In one embodiment, the monosaccharide is an N-acetylgalactosamine (GalNAc). In some embodiments, the carbohydrate conjugate comprises one or more GalNAc derivatives. The GalNAc derivatives may be attached via a linker, e.g., a bivalent or trivalent branched linker. In some embodiments the GalNAc conjugate is conjugated to the 3′ end of the sense strand. In some embodiments, the GalNAc conjugate is conjugated to the iRNA (e.g., to the 3′ end of the sense strand) or antisense polynucleotide agent via a linker, e.g., a linker as described herein.

In some embodiments, the GalNAc conjugate is

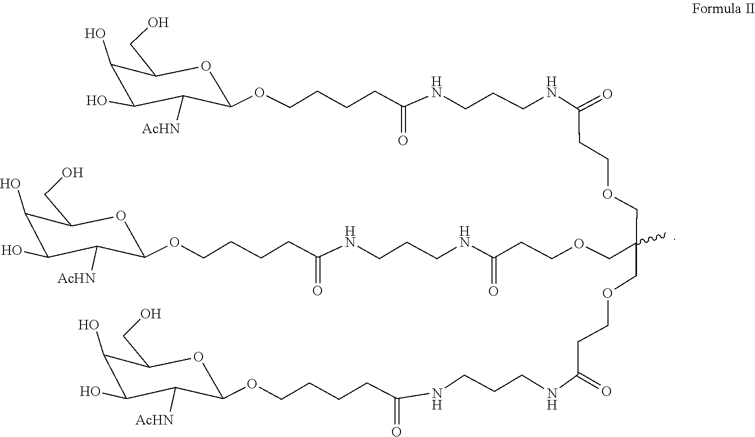 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In some embodiments, the RNAi agent or antisense polynucleotide agent is attached to the carbohydrate conjugate via a linker as shown in the following schematic, wherein X is O or S

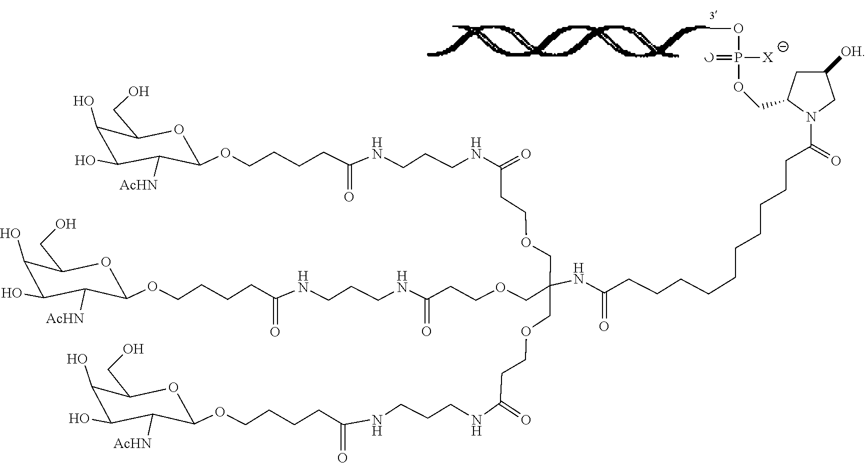

In some embodiments, the RNAi agent or antisense polynucleotide agent is conjugated to L96 as defined in Table 1 and shown below

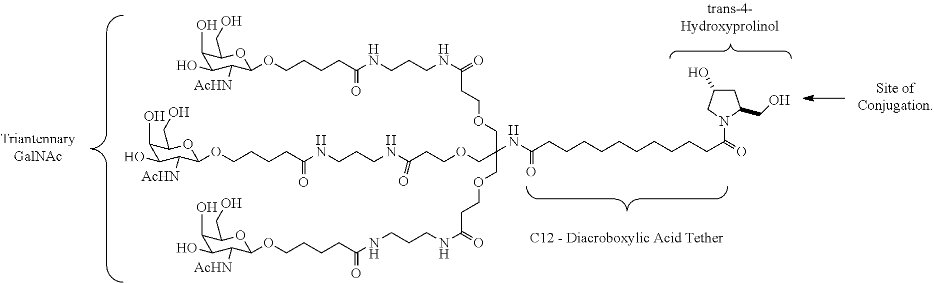

In some embodiments, a carbohydrate conjugate is selected from the group consisting of Formula II to Formula XXIII of WO 2015/050990 or WO 2016/164746, the contents of which are incorporated by reference in their entirety.

In one embodiment, an iRNA or antisense polynucleotide agent described herein is conjugated to a carbohydrate through a linker. Non-limiting examples of iRNA or antisense polynucleotide agent carbohydrate conjugates with linkers include, but are not limited to, Formula XXIV to Formula XXX of WO 2015/050990 or WO 2016/164746, the contents of which are incorporated by reference in their entirety.

In some embodiments, the conjugate or ligand described herein can be attached to an iRNA oligonucleotide with various linkers that can be cleavable or non-cleavable. The term “linker” or “linking group” means an organic moiety that connects two parts of a compound, e.g., covalently attaches two parts of a compound. Exemplary linkers are described, e.g., in WO 2015/050990 or WO 2016/164746, the contents of which are incorporated by reference in their entirety.

The delivery of an iRNA or antisense polynucleotide agent to a subject in need thereof can be achieved in a number of different ways. In vivo delivery can be performed directly by administering a composition comprising an iRNA (e.g. a dsRNA) or antisense polynucleotide agent, to a subject. Alternatively, delivery can be performed indirectly by administering one or more vectors that encode and direct the expression of the iRNA or antisense polynucleotide agent. Exemplary delivery methods are described, e.g., in WO 2015/050990 or WO 2016/164746, the contents of which are incorporated by reference in their entirety.

### VII. Pharmaceutical Compositions

In one aspect, the disclosure provides pharmaceutical compositions containing an iRNA (e.g., a dsRNA) or an antisense polynucleotide agent, as described herein, and a pharmaceutically acceptable carrier.

The phrase “pharmaceutically acceptable” is employed herein to refer to those compounds, materials, compositions, and/or dosage forms which are, within the scope of sound medical judgment, suitable for use in contact with the tissues of human subjects and animal subjects without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum components, such as serum albumin, HDL and LDL; and (22) other non-toxic compatible substances employed in pharmaceutical formulations.

The pharmaceutical composition containing the iRNA (e.g., dsRNA) or antisense polynucleotide agent is useful for treating a LECT2-associated disorder, e.g., a disorder related to the expression or activity of a LECT2 gene (e.g., a LECT2 amyloidosis). Such pharmaceutical compositions are formulated based on the mode of delivery. For example, compositions can be formulated for systemic administration via parenteral delivery, e.g., by intravenous (IV) or subcutaneous (SC) delivery. In some embodiments, a composition provided herein (e.g., an LNP formulation) is formulated for intravenous delivery. In some embodiments, a composition provided herein (e.g., a composition comprising a GalNAc conjugate) is formulated for subcutaneous delivery.

The pharmaceutical compositions featured herein are administered in a dosage sufficient to inhibit expression of a LECT2 gene. In general, a suitable dose of iRNA will be in the range of 0.01 to 200.0 milligrams per kilogram body weight of the recipient per day, generally in the range of 1 to 50 mg per kilogram body weight per day. For example, the dsRNA can be administered at 0.05 mg/kg, 0.5 mg/kg, 1 mg/kg, 1.5 mg/kg, 2 mg/kg, 3 mg/kg, 10 mg/kg, 20 mg/kg, 30 mg/kg, 40 mg/kg, or 50 mg/kg per single dose.

For example, the iRNA (e.g., dsRNA) or antisense polynucleotide agent may be administered at a dose of about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 11, 12, 13, 14, 15, 16, 17, 18, 19, 20, 21, 2, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 67, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 mg/kg. Values and ranges intermediate to the recited values are also intended to be part of this invention.

In another embodiment, the iRNA (e.g., dsRNA) or antisense polynucleotide agent is administered at a dose of about 0.1 to about 100 mg/kg, about 0.25 to about 100 mg/kg, about 0.5 to about 100 mg/kg, about 0.75 to about 100 mg/kg, about 1 to about 100 mg/mg, about 1.5 to about 100 mg/kg, about 2 to about 100 mg/kg, about 2.5 to about 100 mg/kg, about 3 to about 100 mg/kg, about 3.5 to about 100 mg/kg, about 4 to about 100 mg/kg, about 4.5 to about 100 mg/kg, about 5 to about 100 mg/kg, about 7.5 to about 100 mg/kg, about 10 to about 100 mg/kg, about 15 to about 100 mg/kg, about 20 to about 100 mg/kg, about 25 to about 100 mg/kg, about 30 to about 100 mg/kg, about 35 to about 100 mg/kg, about 40 to about 100 mg/kg, about 45 to about 100 mg/kg, about 0.1 to about 50 mg/kg, about 0.25 to about 50 mg/kg, about 0.5 to about 50 mg/kg, about 0.75 to about 50 mg/kg, about 1 to about 50 mg/mg, about 1.5 to about 50 mg/kb, about 2 to about 50 mg/kg, about 2.5 to about 50 mg/kg, about 3 to about 50 mg/kg, about 3.5 to about 50 mg/kg, about 4 to about 50 mg/kg, about 4.5 to about 50 mg/kg, about 5 to about 50 mg/kg, about 7.5 to about 50 mg/kg, about 10 to about 50 mg/kg, about 15 to about 50 mg/kg, about 20 to about 50 mg/kg, about 20 to about 50 mg/kg, about 25 to about 50 mg/kg, about 25 to about 50 mg/kg, about 30 to about 50 mg/kg, about 35 to about 50 mg/kg, about 40 to about 50 mg/kg, about 45 to about 50 mg/kg, about 0.1 to about 45 mg/kg, about 0.25 to about 45 mg/kg, about 0.5 to about 45 mg/kg, about 0.75 to about 45 mg/kg, about 1 to about 45 mg/mg, about 1.5 to about 45 mg/kb, about 2 to about 45 mg/kg, about 2.5 to about 45 mg/kg, about 3 to about 45 mg/kg, about 3.5 to about 45 mg/kg, about 4 to about 45 mg/kg, about 4.5 to about 45 mg/kg, about 5 to about 45 mg/kg, about 7.5 to about 45 mg/kg, about 10 to about 45 mg/kg, about 15 to about 45 mg/kg, about 20 to about 45 mg/kg, about 20 to about 45 mg/kg, about 25 to about 45 mg/kg, about 25 to about 45 mg/kg, about 30 to about 45 mg/kg, about 35 to about 45 mg/kg, about 40 to about 45 mg/kg, about 0.1 to about 40 mg/kg, about 0.25 to about 40 mg/kg, about 0.5 to about 40 mg/kg, about 0.75 to about 40 mg/kg, about 1 to about 40 mg/mg, about 1.5 to about 40 mg/kb, about 2 to about 40 mg/kg, about 2.5 to about 40 mg/kg, about 3 to about 40 mg/kg, about 3.5 to about 40 mg/kg, about 4 to about 40 mg/kg, about 4.5 to about 40 mg/kg, about 5 to about 40 mg/kg, about 7.5 to about 40 mg/kg, about 10 to about 40 mg/kg, about 15 to about 40 mg/kg, about 20 to about 40 mg/kg, about 20 to about 40 mg/kg, about 25 to about 40 mg/kg, about 25 to about 40 mg/kg, about 30 to about 40 mg/kg, about 35 to about 40 mg/kg, about 0.1 to about 30 mg/kg, about 0.25 to about 30 mg/kg, about 0.5 to about 30 mg/kg, about 0.75 to about 30 mg/kg, about 1 to about 30 mg/mg, about 1.5 to about 30 mg/kb, about 2 to about 30 mg/kg, about 2.5 to about 30 mg/kg, about 3 to about 30 mg/kg, about 3.5 to about 30 mg/kg, about 4 to about 30 mg/kg, about 4.5 to about 30 mg/kg, about 5 to about 30 mg/kg, about 7.5 to about 30 mg/kg, about 10 to about 30 mg/kg, about 15 to about 30 mg/kg, about 20 to about 30 mg/kg, about 20 to about 30 mg/kg, about 25 to about 30 mg/kg, about 0.1 to about 20 mg/kg, about 0.25 to about 20 mg/kg, about 0.5 to about 20 mg/kg, about 0.75 to about 20 mg/kg, about 1 to about 20 mg/mg, about 1.5 to about 20 mg/kb, about 2 to about 20 mg/kg, about 2.5 to about 20 mg/kg, about 3 to about 20 mg/kg, about 3.5 to about 20 mg/kg, about 4 to about 20 mg/kg, about 4.5 to about 20 mg/kg, about 5 to about 20 mg/kg, about 7.5 to about 20 mg/kg, about 10 to about 20 mg/kg, or about 15 to about 20 mg/kg. Values and ranges intermediate to the recited values are also intended to be part of this invention.

For example, the iRNA (e.g., dsRNA) or antisense polynucleotide agent may be administered at a dose of about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 11, 12, 13, 14, 15, 16, 17, 18, 19, 20, 21, 2, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 67, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 mg/kg. Values and ranges intermediate to the recited values are also intended to be part of this invention.

In another embodiment, the iRNA (e.g., dsRNA) or antisense polynucleotide agent is administered at a dose of about 0.1 to about 100 mg/kg, about 0.25 to about 100 mg/kg, about 0.5 to about 100 mg/kg, about 0.75 to about 100 mg/kg, about 1 to about 100 mg/mg, about 1.5 to about 100 mg/kg, about 2 to about 100 mg/kg, about 2.5 to about 100 mg/kg, about 3 to about 100 mg/kg, about 3.5 to about 100 mg/kg, about 4 to about 100 mg/kg, about 4.5 to about 100 mg/kg, about 5 to about 100 mg/kg, about 7.5 to about 100 mg/kg, about 10 to about 100 mg/kg, about 15 to about 100 mg/kg, about 20 to about 100 mg/kg, about 25 to about 100 mg/kg, about 30 to about 100 mg/kg, about 35 to about 100 mg/kg, about 40 to about 100 mg/kg, about 45 to about 100 mg/kg, about 0.5 to about 50 mg/kg, about 0.75 to about 50 mg/kg, about 1 to about 50 mg/mg, about 1.5 to about 50 mg/kg, about 2 to about 50 mg/kg, about 2.5 to about 50 mg/kg, about 3 to about 50 mg/kg, about 3.5 to about 50 mg/kg, about 4 to about 50 mg/kg, about 4.5 to about 50 mg/kg, about 5 to about 50 mg/kg, about 7.5 to about 50 mg/kg, about 10 to about 50 mg/kg, about 15 to about 50 mg/kg, about 20 to about 50 mg/kg, about 20 to about 50 mg/kg, about 25 to about 50 mg/kg, about 25 to about 50 mg/kg, about 30 to about 50 mg/kg, about 35 to about 50 mg/kg, about 40 to about 50 mg/kg, about 45 to about 50 mg/kg, about 0.5 to about 45 mg/kg, about 0.75 to about 45 mg/kg, about 1 to about 45 mg/mg, about 1.5 to about 45 mg/kb, about 2 to about 45 mg/kg, about 2.5 to about 45 mg/kg, about 3 to about 45 mg/kg, about 3.5 to about 45 mg/kg, about 4 to about 45 mg/kg, about 4.5 to about 45 mg/kg, about 5 to about 45 mg/kg, about 7.5 to about 45 mg/kg, about 10 to about 45 mg/kg, about 15 to about 45 mg/kg, about 20 to about 45 mg/kg, about 20 to about 45 mg/kg, about 25 to about 45 mg/kg, about 25 to about 45 mg/kg, about 30 to about 45 mg/kg, about 35 to about 45 mg/kg, about 40 to about 45 mg/kg, about 0.5 to about 40 mg/kg, about 0.75 to about 40 mg/kg, about 1 to about 40 mg/mg, about 1.5 to about 40 mg/kb, about 2 to about 40 mg/kg, about 2.5 to about 40 mg/kg, about 3 to about 40 mg/kg, about 3.5 to about 40 mg/kg, about 4 to about 40 mg/kg, about 4.5 to about 40 mg/kg, about 5 to about 40 mg/kg, about 7.5 to about 40 mg/kg, about 10 to about 40 mg/kg, about 15 to about 40 mg/kg, about 20 to about 40 mg/kg, about 20 to about 40 mg/kg, about 25 to about 40 mg/kg, about 25 to about 40 mg/kg, about 30 to about 40 mg/kg, about 35 to about 40 mg/kg, about 0.5 to about 30 mg/kg, about 0.75 to about 30 mg/kg, about 1 to about 30 mg/mg, about 1.5 to about 30 mg/kb, about 2 to about 30 mg/kg, about 2.5 to about 30 mg/kg, about 3 to about 30 mg/kg, about 3.5 to about 30 mg/kg, about 4 to about 30 mg/kg, about 4.5 to about 30 mg/kg, about 5 to about 30 mg/kg, about 7.5 to about 30 mg/kg, about 10 to about 30 mg/kg, about 15 to about 30 mg/kg, about 20 to about 30 mg/kg, about 20 to about 30 mg/kg, about 25 to about 30 mg/kg, about 0.5 to about 20 mg/kg, about 0.75 to about 20 mg/kg, about 1 to about 20 mg/mg, about 1.5 to about 20 mg/kb, about 2 to about 20 mg/kg, about 2.5 to about 20 mg/kg, about 3 to about 20 mg/kg, about 3.5 to about 20 mg/kg, about 4 to about 20 mg/kg, about 4.5 to about 20 mg/kg, about 5 to about 20 mg/kg, about 7.5 to about 20 mg/kg, about 10 to about 20 mg/kg, or about 15 to about 20 mg/kg. In one embodiment, the antisense polynucleotide agent is administered at a dose of about 0.5 mg/kg to about 10 mg/kg. Values and ranges intermediate to the recited values are also intended to be part of this invention.

For example, subjects can be administered, e.g., subcutaneously or intravenously, a single therapeutic amount of the iRNA (e.g., dsRNA) or antisense polynucleotide agent, such as about 0.1, 0.125, 0.15, 0.175, 0.2, 0.225, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95, 0.975, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 31, 32, 33, 34, 34, 35, 36, 37, 38, 39, 40, 41, 42, 43, 44, 45, 46, 47, 48, 49, 50, 51, 52, 53, 54, 55, 56, 67, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 mg/kg. Values and ranges intermediate to the recited values are also intended to be part of this invention.

In some embodiments, subjects are administered, e.g., subcutaneously or intravenously, multiple doses of a therapeutic amount of iRNA (e.g., dsRNA) or antisense polynucleotide agent, such as a dose about 0.1, 0.125, 0.15, 0.175, 0.2, 0.225, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95, 0.975, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 31, 32, 33, 34, 34, 35, 36, 37, 38, 39, 40, 41, 42, 43, 44, 45, 46, 47, 48, 49, 50, 51, 52, 53, 54, 55, 56, 67, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 mg/kg. A multi-dose regimen may include administration of a therapeutic amount of antisense polynucleotide agent daily, such as for two days, three days, four days, five days, six days, seven days, or longer.

In other embodiments, subjects are administered, e.g., subcutaneously or intravenously, a repeat dose of a therapeutic amount of iRNA (e.g., dsRNA) or antisense polynucleotide agent, such as a dose about 0.1, 0.125, 0.15, 0.175, 0.2, 0.225, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95, 0.975, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 31, 32, 33, 34, 34, 35, 36, 37, 38, 39, 40, 41, 42, 43, 44, 45, 46, 47, 48, 49, 50, 51, 52, 53, 54, 55, 56, 67, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 mg/kg. A repeat-dose regimen may include administration of a therapeutic amount of iRNA (e.g., dsRNA) or antisense polynucleotide agent on a regular basis, such as every other day, every third day, every fourth day, twice a week, once a week, every other week, or once a month.

The pharmaceutical composition can be administered by intravenous infusion over a period of time, such as over a 5, 6, 7, 8, 9, 10, 11, 12, 13, 14, 15, 16, 17, 18, 19, 20, and 21, 22, 23, 24, or about a 25 minute period. The administration may be repeated, for example, on a regular basis, such as weekly, biweekly (i.e., every two weeks) for one month, two months, three months, four months or longer. After an initial treatment regimen, the treatments can be administered on a less frequent basis. For example, after administration weekly or biweekly for three months, administration can be repeated once per month, for six months or a year or longer. The pharmaceutical composition may be administered once daily, or as two, three, or more sub-doses at appropriate intervals throughout the day, or even using continuous infusion or delivery through a controlled release formulation. In that case, the iRNA (e.g., dsRNA) or antisense polynucleotide agent contained in each sub-dose must be correspondingly smaller in order to achieve the total daily dosage. The dosage unit can also be compounded for delivery over several days, e.g., using a conventional sustained release formulation which provides sustained release of the iRNA (e.g., dsRNA) or antisense polynucleotide agent over a several day period. Sustained release formulations are well known in the art and are particularly useful for delivery of agents at a particular site, such as can be used with the agents of the present invention. In this embodiment, the dosage unit contains a corresponding multiple of the daily dose.

The effect of a single dose on LECT2 levels can be long lasting, such that subsequent doses are administered at not more than 3, 4, or 5 day intervals, or at not more than 1, 2, 3, or 4 week intervals.

The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual iRNAs encompassed by the invention can be made using conventional methodologies or on the basis of in vivo testing using a suitable animal model.

A suitable animal model, e.g., a mouse containing a transgene expressing human LECT2, can be used to determine the therapeutically effective dose and/or an effective dosage regimen administration of LECT2 siRNA.

The pharmaceutical compositions described herein may be administered in a number of ways depending upon whether local or systemic treatment is desired and upon the area to be treated. Administration may be topical (e.g., by a transdermal patch), pulmonary, e.g., by inhalation or insufflation of powders or aerosols, including by nebulizer; intratracheal, intranasal, epidermal and transdermal, oral or parenteral. Parenteral administration includes intravenous, intraarterial, subcutaneous, intraperitoneal or intramuscular injection or infusion; subdermal, e.g., via an implanted device; or intracranial, e.g., by intraparenchymal, intrathecal or intraventricular, administration.

The iRNA or antisense polynucleotide agent can be delivered in a manner to target a particular tissue, such as a tissue that produces erythrocytes. For example, the iRNA or antisense polynucleotide agent can be delivered to bone marrow, liver (e.g., hepatocytes of liver), lymph glands, spleen, lungs (e.g., pleura of lungs) or spine. In one embodiment, the iRNA or antisense polynucleotide agent is delivered to bone marrow.

Suitable formulations include those in which the iRNAs (e.g., dsRNAs) or antisense polynucleotide agents described herein are in admixture with a delivery agent such as lipids, liposomes, fatty acids, fatty acid esters, steroids, chelating agents and surfactants. Suitable lipids and liposomes include neutral (e.g., dioleoylphosphatidyl DOPE ethanolamine, dimyristoylphosphatidyl choline DMPC, distearolyphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA).

In some embodiments, the iRNAs or antisense polynucleotide agents described herein can be encapsulated within liposomes or can form complexes thereto, in particular to cationic liposomes. Alternatively, the iRNAs or antisense polynucleotide agents can be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₂₀ alkyl ester (e.g., isopropylmyristate IPM), monoglyceride, diglyceride or pharmaceutically acceptable salt thereof).

An iRNA (e.g., dsRNA) or antisense polynucleotide agent for use in the compositions and methods described herein can be formulated for delivery in a membranous molecular assembly, e.g., a liposome or a micelle. As used herein, the term “liposome” refers to a vesicle composed of amphiphilic lipids arranged in at least one bilayer, e.g., one bilayer or a plurality of bilayers.

Exemplary liposomes containing iRNAs (e.g., a dsRNAs) targeting LECT2 are described, e.g., in International Application Publication No. WO 2015/050990, the content of which is incorporated by reference in its entirety. Exemplary liposomes containing antisense polynucleotide agents targeting LECT2 are described, e.g., in International Application Publication No. WO 2016/164746, the content of which is incorporated by reference in its entirety.

In some embodiments, compositions described herein can be formulated as emulsions. Emulsions are typically heterogeneous systems of one liquid dispersed in another in the form of droplets usually exceeding 0.1 μm in diameter (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301).

In other embodiments, the compositions described herein are formulated as microemulsions. A microemulsion can be defined as a system of water, oil and amphiphile which is a single optically isotropic and thermodynamically stable liquid solution (see e.g., Ansel's Pharmaceutical Dosage Forms and Drug Delivery Systems, Allen, LV., Popovich N G., and Ansel H C., 2004, Lippincott Williams & Wilkins (8th ed.), New York, N.Y.; Rosoff, in Pharmaceutical Dosage Forms, Lieberman, Rieger and Banker (Eds.), 1988, Marcel Dekker, Inc., New York, N.Y., volume 1, p. 245).

In other embodiments, the iRNAs (e.g., dsRNAs) or antisense polynucleotide agents described herein are incorporated into particles, e.g., microparticle. Microparticles can be produced by spray-drying, but may also be produced by other methods including lyophilization, evaporation, fluid bed drying, vacuum drying, or a combination of these techniques.

In some embodiments, the compositions described herein employ various penetration enhancers to effect the efficient delivery of iRNAs (e.g., dsRNAs) or antisense polynucleotide agents, to the skin of animals. Penetration enhancers can be classified as belonging to one of five broad categories, i.e., surfactants, fatty acids, bile salts, chelating agents, and non-chelating non-surfactants (see e.g., Malmsten, M. Surfactants and polymers in drug delivery, Informa Health Care, New York, N.Y., 2002; Lee et al., Critical Reviews in Therapeutic Drug Carrier Systems, 1991, p. 92). Exemplary penetration enhancers are described, e.g., in WO 2015/050990 and WO 2016/164746, which are incorporated by reference it their entirety.

Certain compositions described herein also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation.

A “pharmaceutical carrier” or “excipient” is a pharmaceutically acceptable solvent, suspending agent or any other pharmacologically inert vehicle for delivering one or more nucleic acids to an animal. The excipient can be liquid or solid and is selected, with the planned manner of administration in mind, so as to provide for the desired bulk, consistency, etc., when combined with a nucleic acid and the other components of a given pharmaceutical composition.

The compositions described herein can additionally contain other adjunct components conventionally found in pharmaceutical compositions, at their art-established usage levels.

In addition to their administration, as discussed above, the iRNAs or antisense polynucleotide agents described herein can be administered in combination with other known agents effective in treatment of pathological processes mediated by LECT2 gene expression. In any event, the administering physician can adjust the amount and timing of antisense polynucleotide agent administration on the basis of results observed using standard measures of efficacy known in the art or described herein.

Other exemplary pharmaceutical compositions containing iRNAs (e.g., a dsRNAs) targeting LECT2 are described, e.g., in International Application Publication No. WO 2015/050990, the content of which is incorporated by reference in its entirety. Other exemplary pharmaceutical compositions containing antisense polynucleotide agents targeting LECT2 are described, e.g., in International Application Publication No. WO 2016/164746, the content of which is incorporated by reference in its entirety.

### VI. Methods of Treating LECT2-associated Disorders

The nucleic acid agents (e.g., dsRNAs or antisense polynucleotide agents) described herein can be used to inhibit LECT2 expression and/or to treat a disease, disorder, or pathological process that is associated with LECT2 (e.g., related to LECT2 expression) in a subject.

In one aspect, a method of treatment of a LECT2-associated disorder is provided, the method comprising administering a nucleic acid agent (e.g., a dsRNA or antisense polynucleotide agent) disclosed herein to a subject in need thereof. In some embodiments, the nucleic acid agent (e.g., dsRNA or antisense polynucleotide agent) inhibits (e.g., decreases) LECT2 expression in the subject.

In some embodiments, the subject has an aberrant (e.g., elevated) level of LECT2 expression, e.g., as determined by a method described herein. In some embodiments, the treatment is responsive to the determination that the subject has an aberrant (e.g., elevated) level of LECT2 expression. In some embodiments, the subject is identified for the treatment by the determination of an aberrant (e.g., elevated) level of LECT2 expression.

In some embodiments, the subject has, or is at risk of having, a LECT2-associated disorder, e.g., as determined by a method described herein. In some embodiments, the treatment is responsive to the determination that the subject has, or is at risk of having, a LECT2-associated disorder.

In some embodiments, the method includes determining the level of a LECT2 RNA in the subject, in accordance with a method described herein. In some embodiments, the method includes determining the level of a LECT2 RNA, or a cleavage product thereof, in the subject, in accordance with a method described herein. In some embodiments, the level of a LECT2 RNA, or a cleavage product thereof, is determined in a bodily fluid sample (e.g., blood (e.g., serum or plasma) or urine), by a method described herein.

The nucleic acid agents disclosed herein (e.g., a dsRNA or antisense polynucleotide agent disclosed herein) can be used, e.g., in the manufacture of a medicament, for treating, a LECT2-associated disorder described herein.

As used herein, a “LECT2-associated disorder,” “LECT2-associated disease,” “LECT2-associated pathological process” or the like includes any condition, disorder or disease in which an activity or expression of LECT2 is altered, e.g., relative to a normal level. In some embodiments, the LECT2-associated disorder is a disorder related to LECT2 expression. In certain embodiments, the expression of LECT2 is increased. In other embodiments, the expression of LECT2 is decreased. In some embodiments, the decrease or increase in LECT2 expression is detectable in a bodily fluid sample from the subject (e.g. in a blood (e.g., serum or plasma) or urine sample) of the subject. In some embodiments, the decrease or increase in LECT2 expression is detectable in a tissue sample from the subject (e.g., in a liver sample). In some embodiments, an LECT2 activity (e.g., LECT2 deposition) is altered. The decrease or increase of an activity or expression may be assessed relative to the level observed in the same individual prior to the development of the disorder or relative to other individual(s) who do not have the disorder. The decrease or increase may be limited to a particular organ, tissue, or region of the body (e.g., liver). In one embodiment, the LECT2-associated disorder is amyloidosis, e.g., LECT2 amyloidosis (ALECT2).

As used herein, a “subject” to be treated according to the methods described herein, includes a human or non-human animal, e.g., a mammal. The mammal may be, for example, a rodent (e.g., a rat or mouse) or a primate (e.g., a monkey). In some embodiments, the subject is a human.

A “subject in need thereof” includes a subject having, suspected of having, or at risk of developing a LECT2-associated disorder. In some embodiments, the subject has, or is suspected of having, a LECT2-associated disorder (e.g., an ALECT2-associated disorder). In some embodiments, the subject is at risk of having a LECT2-associated disorder (e.g., an ALECT2-associated disorder).

In some embodiments, the subject is an animal that serves as a model for a LECT2-associated disorder, e.g., a LECT2 amyloidosis.

LECT2 Amyloidosis

In some embodiments, the LECT2-associated disorder is an amyloidosis, e.g., a LECT2 amyloidosis. LECT2 amyloidosis has been described in several clinical studies. See, e.g., Benson, M. D. et al (2008) Kidney International, 74: 218-222; Murphy, C. L. et al. (2010) Am J Kidney Dis, 56(6):1100-1107; Larsen, C. P. et al. (2010) Kidney Int., 77(9):816-819; Holanda, D. G. et al. (20011) Nephrol. Dial. Transplant., 26 (1): 373-376; and Sethi, S. et al. (2012) Kidney International 82, 226-234 (hereinafter Sethi et al.).

Clinical and pathological features of LECT2 amyloidosis mimic those of amyloid light chain (AL) amyloidosis. These symptoms include, e.g., symptoms of kidney disease and renal failure, e.g., fluid retention, swelling, and shortness of breath. Amyloidosis may affect the heart, peripheral nervous system, gastrointestinal tract, blood, lungs and skin. Heart complications include, e.g., heart failure and irregular heartbeat. Other symptoms include, e.g., stroke, gastrointestinal disorders, enlarged liver, diminished spleen function, diminished function of the adrenal and other endocrine glands, skin color change or growths, lung problems, bleeding and bruising problems, fatigue and weight loss. In some embodiments, the methods described herein are associated with improvement in one or more symptoms described herein.

Methods for diagnosis of amyloidosis, e.g., LECT2 amyloidosis, are described, e.g., in Leung, N. et al. (2010) Blood, published online Sep. 4, 2012; DOI 10.1182/blood-2012-03-413682; Shiller, S. M. et al. (2011). Laboratory Methods for the Diagnosis of Hereditary Amyloidoses, Amyloidosis—Mechanisms and Prospects for Therapy, Dr. Svetlana Sarantseva (Ed.), ISBN: 978-953-307-253-1; Sethi et al. (see above) and in U.S. Patent Application Publication No. 20100323381.

Based on the results provided by Sethi et al., LECT2 amyloidosis accounts for a significant percentage of cases of renal amyloidosis. See Table 1 of Sethi et al., which shows that 26 out of 127 cases of renal amyloidosis studied by laser microdissection and mass spectrometry of renal biopsy and/or nephrectomy specimens were determined to be of the LECT2 amyloid type. Sethi et al. further report that apolipoprotein E protein and serum amyloid P component (SAP) were also present in all cases of LECT2 amyloidosis.

In some embodiments, the amyloidosis, e.g., the LECT2 amyloidosis, involves systemic amyloid deposition. In some embodiments, the amyloidosis, e.g., the LECT2 amyloidosis, is localized entirely or predominately to a particular tissue or organ (e.g., to the liver).

In some embodiments, the amyloidosis, e.g., the LECT2 amyloidosis, is hereditary.

In some embodiments, a LECT2 amyloidosis is diagnosed using analysis of a sample from the subject (e.g., a biopsy sample). In some embodiments, the biopsy sample is a renal biopsy. In some embodiments, the sample is a nephrectomy sample. In some embodiments, the sample is from a liver biopsy or from other resected liver tissue. In some embodiments, the sample is analyzed using methods selected from one or more of immunohistochemistry, LECT2 immunoassay, electron microscopy, laser microdissection, and mass spectrometry. In some embodiments, the LECT2 amyloidosis is diagnosed using laser microdissection and mass spectrometry.

In some embodiments, the amyloidosis, e.g., the LECT2 amyloidosis, affects the kidney, e.g., involves amyloid deposition in the kidney. In some embodiments, kidney function is compromised as a result of the amyloidosis. In some embodiments, the subject suffers from one or more of fluid retention, swelling, and shortness of breath. In some embodiments, the subject has renal insufficiency. In some embodiments, the subject has nephrotic syndrome. In some embodiments, the subject suffers from proteinuria. In some embodiments, the subject has renal failure.

In some embodiments, the amyloidosis, e.g., the LECT2 amyloidosis, affects the liver, e.g., involves amyloid deposition in the liver. In some embodiments, liver function is compromised as a result of the amyloidosis. In some embodiments, the subject has hepatitis, e.g., chronic hepatitis. In some embodiments, the hepatitis is a viral hepatitis.

LECT2 amyloidosis has been found to be particularly prevalent in Mexican Americans and has also been associated with homozygosity for the G allele of the LECT2 gene that encodes valine at position 40 in the mature protein (amino acid 58 in the unprocessed protein). See, e.g., Benson, M. D. et al. (2008) Kidney International, 74: 218-222; Murphy, C. L. et al. (2010) Am J Kidney Dis, 56(6):1100-1107.

In some embodiments, the subject is of Mexican descent. In some embodiments, the subject is a Mexican American.

In some embodiments, the subject carries the G allele of the LECT2 gene that encodes valine at position 40 in the mature protein (amino acid 58 in the unprocessed protein). In some embodiments, the subject is homozygous for the G allele (G/G genotype). In some embodiments, a LECT2 protein expressed in the subject has valine at position 40 in the mature protein (or at amino acid 58 in the unprocessed protein).

In some embodiments, the method decreases LECT2 expression. In some embodiments, the decrease in LECT2 expression is assessed relative to the level in the same individual prior to the treatment. In some embodiments, the method is shown to decrease LECT2 expression by comparing the levels of LECT2 expression in a treated subject (or group of subjects) with the levels in a control subject (or group of subjects), e.g., an untreated subject (or group of subjects) or a subject (or group of subjects) treated with a control treatment (e.g., an iRNA (e.g., a dsRNA) that does not target LECT2).

In some embodiments, the method reduces amyloid deposition, e.g., deposition of amyloid comprising a LECT2 protein or a portion thereof. In some embodiments, the protein is a wild type protein. In some embodiments, the protein is a human LECT2 protein, or a portion thereof, that includes valine at position 40 (position 40 of the mature, secreted protein, or at amino acid 58 in the unprocessed protein, as described herein). In some embodiments, the method decreases the size, number, and/or extent of amyloid deposits.

In some embodiments, the method decreases one or more symptoms associated with amyloid deposition.

In some embodiments, the therapeutic nucleic acid (e.g., a dsRNA or antisense polynucleotide agent) is administered in a form that targets the therapeutic nucleic acid to a particular organ or tissue to inhibit amyloid deposition in the organ or tissue.

In some embodiments, the therapeutic nucleic acid (e.g., a dsRNA or antisense polynucleotide agent) is targeted to the liver. In some embodiments, the therapeutic nucleic acid is conjugated to a ligand, e.g., a GalNAc ligand (e.g., a GalNAc ligand as described herein) that targets the dsRNA to the liver (e.g., to hepatocytes).

Also provided herein is a method of reducing amyloid deposition, the method comprising administering a therapeutic nucleic acid (e.g., a dsRNA or antisense polynucleotide agent) as disclosed herein to a subject in need thereof (e.g., a subject having, suspected of having, or at risk for developing a LECT2 amyloidosis). In some embodiments, the method decreases (e.g., prevents or diminishes) the size, number, and/or extent of amyloid deposits. The size, number, and/or extent of amyloid deposits may be assessed using any method known in the art (e.g., immunoassay, immunohistochemistry, mass spectrometry). The reduction of amyloid deposition may involve a decrease in amyloid deposition (e.g., size, number, and/or extent of amyloid deposits) of at least 5%, 10%, 15%, 20%, 25%, 30%, 35%, 40%, 45%, 50% or more.

In the methods provided herein, the iRNA (e.g., dsRNA) or antisense polynucleotide agent and compositions thereof are administered in a therapeutically effective amount. Therapeutic effects of administration of a LECT2 siRNA can be established, for example, by comparison with an appropriate control. For example, inhibition of amyloid deposition may be established, for example, in a group of patients with amyloidosis (e.g., LECT2 amyloidosis) by comparison of any appropriate parameter (e.g., a parameter assessing the size, number, or extent of amyloid deposition) with the same parameter in an appropriate control group. A control group (e.g., a group of similar individuals or the same group of individuals in a crossover design) may include, for example, an untreated population, a population that has been treated with a conventional treatment; a population that has been treated with placebo or a non-targeting iRNA; and the like.

Rheumatoid Arthritis

In one embodiment, the LECT2-associated disorder is rheumatoid arthritis. For example, in a Japanese population, it was found that possession of one A allele of the LECT2 gene that encodes isoleucine at position 40 in the mature protein (or amino acid 58 in the unprocessed protein) was found to increase the overall risk of developing rheumatoid arthritis. Possessing two A alleles was strongly associated with disease severity. See Kameoka, Y. et al. (2000) Arth Rheum, 43(6):1419-20.

In one embodiment, the dsRNA or antisense polynucleotide agent inhibits LECT2 expression in a subject having rheumatoid arthritis. In some such embodiments, the dsRNA or antisense polynucleotide agent inhibits LECT2 expression in synovial tissue and/or in synovial fluid-derived cells (e.g., mononuclear cells and fibroblasts). In some embodiments, the dsRNA or antisense polynucleotide targets an mRNA that encodes isoleucine at position 40 in the mature protein (amino acid 58 in the unprocessed protein).

Liver Injury

In one embodiment, the LECT2-associated disorder is liver injury. For example, LECT2 expression can increase during acute liver injury.

In one embodiment, the dsRNA or antisense polynucleotide agent inhibits LECT2 expression in a subject having liver injury. In some embodiments, the dsRNA or antisense polynucleotide agent inhibits LECT2 expression in the liver.

Combination Therapies

In some embodiments, a nucleic acid agent (e.g., a dsRNA or antisense polynucleotide agent) disclosed herein is administered in combination with a second therapy (e.g., one or more additional therapies) known to be effective in treating a LECT2-associated disorder (e.g., a LECT2 amyloidosis) or a symptom of such a disorder. The nucleic acid agent may be administered before, after, or concurrent with the second therapy. In some embodiments, the nucleic acid agent is administered before the second therapy. In some embodiments, the nucleic acid agent is administered after the second therapy. In some embodiments, the nucleic acid agent is administered concurrent with the second therapy.

The second therapy may be an additional therapeutic agent. The nucleic acid agent and the additional therapeutic agent can be administered in combination in the same composition or the additional therapeutic agent can be administered as part of a separate composition.

In some embodiments, the second therapy is a non-nucleic acid agent therapeutic agent that is effective to treat the disorder or symptoms of the disorder.

In some embodiments, the disorder to be treated by the compositions or methods disclosed herein is a LECT2 amyloidosis that affects kidney function, e.g., through amyloid deposition in the kidney. In some such embodiments, the nucleic acid agent is administered in conjunction with a therapy that supports kidney function (e.g., dialysis, a diuretic, an angiotensin converting enzyme (ACE) inhibitor, an angiotensin receptor blocker (ARB), or dialysis).

In some embodiments, the disorder to be treated by the compositions or methods disclosed herein is a LECT2 amyloidosis involving amyloid deposits in the liver. In some such embodiments, the iRNA is administered in conjunction with a therapy that supports liver function.

In some embodiments, the disorder to be treated by the compositions or methods disclosed herein is a LECT2 amyloidosis, and the nucleic acid agent is administered in conjunction with removal of all or part of the organ(s) affected by the amyloidosis (e.g., resection of all or part of kidney or liver tissue affected by the amyloidosis). The removal is optionally conducted in conjunction with a replacement of all or part of the organ removed (e.g., in conjunction with a kidney or liver organ transplant).

Administration Dosages, Routes, and Timing

A subject (e.g., a human subject, e.g., a patient) can be administered a therapeutic amount of a nucleic acid agent (e.g., a dsRNA or antisense polynucleotide agent). The therapeutic amount can be, e.g., 0.05-50 mg/kg. For example, the therapeutic amount can be 0.05, 0.1, 0.2, 0.3, 0.4, 0.5, 0.6, 0.7, 0.8, 0.9, 1.0, 1.5, 2.0, or 2.5, 3.0, 3.5, 4.0, 4.5, 5, 10, 15, 20, 25, 30, 35, 40, 45, or 50 mg/kg of the nucleic acid agent. In certain embodiments, the nucleic acid agent is a dsRNA. In other embodiments, the nucleic acid agent is an antisense polynucleotide agent.

In some embodiments, the nucleic acid agent is formulated for delivery to a target organ, e.g., to the liver.

In some embodiments, the nucleic acid agent is formulated as a lipid formulation, e.g., an LNP formulation as described herein. In some such embodiments, the therapeutic amount is 0.05-5 mg/kg, e.g., 0.05, 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 mg/kg dsRNA or antisense polynucleotide agent. In some embodiments, the lipid formulation, e.g., LNP formulation, is administered intravenously. In some embodiments, the dsRNA or antisense polynucleotide agent is formulated as an LNP formulation and is administered (e.g., intravenously administered) at a dose of 0.1 to 0.5 mg/kg.

In some embodiments, the nucleic acid agent is administered by intravenous infusion over a period of time, such as over a 5 minute, 10 minute, 15 minute, 20 minute, or 25 minute period.

In some embodiments, the nucleic acid agent is in the form of a GalNAc conjugate as described herein. In some such embodiments, the therapeutic amount is 0.5-50 mg, e.g., 0.5, 0.6, 0.7, 0.8, 0.9, 1.0, 1.5, 2.0, 2.5, 3.0, 3.5, 4.0, 4.5, 5.0, 6, 7, 8, 9, 10, 15, 20, 25, 30, 35, 40, 45, or 50 mg/kg dsRNA. In some embodiments, the GalNAc conjugate is administered subcutaneously. In some embodiments, nucleic acid agent is in the form of a GalNAc conjugate and is administered (e.g., subcutaneously administered) at a dose of 1 to 10 mg/kg.

In some embodiments, the administration is repeated, for example, on a regular basis, such as, daily, biweekly (i.e., every two weeks) for one month, two months, three months, four months or longer. After an initial treatment regimen, the treatments can be administered on a less frequent basis. For example, after administration biweekly for three months, administration can be repeated once per month, for six months or a year or longer.

In some embodiments, the nucleic acid agent is administered in two or more doses. In some embodiments, the number or amount of subsequent doses is dependent on the achievement of a desired effect, e.g., inhibition of amyloid deposition, or the achievement of a therapeutic or prophylactic effect, e.g., reduction or prevention of one or more symptoms associated with the disorder.

In some embodiments, the nucleic acid agent is administered according to a schedule. For example, the iRNA agent may be administered once per week, twice per week, three times per week, four times per week, or five times per week. In some embodiments, the schedule involves regularly spaced administrations, e.g., hourly, every four hours, every six hours, every eight hours, every twelve hours, daily, every 2 days, every 3 days, every 4 days, every 5 days, weekly, biweekly, or monthly. In some embodiments, the nucleic acid agent is administered at the frequency required to achieve a desired effect.

In some embodiments, the schedule involves closely spaced administrations followed by a longer period of time during which the agent is not administered. For example, the schedule may involve an initial set of doses that are administered in a relatively short period of time (e.g., about every 6 hours, about every 12 hours, about every 24 hours, about every 48 hours, or about every 72 hours) followed by a longer time period (e.g., about 1 week, about 2 weeks, about 3 weeks, about 4 weeks, about 5 weeks, about 6 weeks, about 7 weeks, or about 8 weeks) during which the nucleic acid agent is not administered. In one embodiment, the nucleic acid agent is initially administered hourly and is later administered at a longer interval (e.g., daily, weekly, biweekly, or monthly). In another embodiment, the nucleic acid agent is initially administered daily and is later administered at a longer interval (e.g., weekly, biweekly, or monthly). In certain embodiments, the longer interval increases over time or is determined based on the achievement of a desired effect.

Before administration of a full dose of the nucleic acid agent, patients can be administered a smaller dose, such as a 5% infusion dose, and monitored for adverse effects, such as an allergic reaction, or for elevated lipid levels or blood pressure. In another example, the patient can be monitored for unwanted effects.

### VII. Methods for Modulating Expression of a LECT2 Gene

In yet another aspect, the disclosure provides a method for modulating (e.g., inhibiting) the expression of a LECT2 gene, e.g., in a cell or in a subject.

In some embodiments, the cell or subject has an aberrant (e.g., elevated) level of LECT2 expression, e.g., as determined by a method described herein. In some embodiments, the subject has, or is at risk of having, a LECT2-associated disorder (e.g., an ALECT2-associated disorder), e.g., as determined by a method described herein.

In some embodiments, the method includes determining the level of a LECT2 RNA in the cell or subject, in accordance with a method described herein. In some embodiments, the method includes determining the level of a LECT2 RNA, or a cleavage product thereof, in the cell or subject, in accordance with a method described herein. In some embodiments, the level of a LECT2 RNA, or a cleavage product thereof, is determined in a bodily fluid sample (e.g., blood (e.g., serum or plasma) or urine), by a method described herein.

In some embodiments, the cell is ex vivo, in vitro, or in vivo. In some embodiments, the cell is in the liver (e.g., a hepatocyte). In some embodiments, the cell is in a subject (e.g., a mammal, such as, for example, a human).

In one embodiment, the method includes contacting the cell with a dsRNA or antisense polynucleotide agent as described herein, in an amount effective to decrease the expression of a LECT2 gene in the cell. “Contacting,” as used herein, includes directly contacting a cell, as well as indirectly contacting a cell. For example, a cell within a subject may be contacted when a composition comprising an iRNA or antisense polynucleotide agent is administered (e.g., intravenously or subcutaneously) to the subject.

In some embodiments, the expression of LECT2 is inhibited by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, the iRNA has an IC₅₀ in the range of 0.001-0.01 nM, 0.001-0.10 nM, 0.001-1.0 nM, 0.001-10 nM, 0.01-0.05 nM, 0.01-0.50 nM, 0.02-0.60 nM, 0.01-1.0 nM, 0.01-1.5 nM, 0.01-10 nM. The IC₅₀ value may be normalized relative to an appropriate control value, e.g., the IC₅₀ of a non-targeting iRNA. The expression of a LECT2 gene may be assessed based on the level of expression of a LECT2 mRNA, a LECT2 protein, or the level of another parameter functionally linked to the level of expression of a LECT2 gene.

In some embodiments, the method includes introducing into the cell an iRNA or antisense polynucleotide agent as described herein and maintaining the cell for a time sufficient to obtain degradation of the mRNA transcript of a LECT2 gene, thereby inhibiting the expression of the LECT2 gene in the cell.

In one embodiment, the method includes administering a composition described herein, e.g., a composition comprising an iRNA or antisense polynucleotide agent that targets LECT2, to the mammal such that expression of the target LECT2 gene is decreased, such as for an extended duration, e.g., at least two, three, four days or more, e.g., one week, two weeks, three weeks, or four weeks or longer. In some embodiments, the decrease in expression of LECT2 is detectable within 1 hour, 2 hours, 4 hours, 8 hours, 12 hours, or 24 hours of the first administration.

In another embodiment, the method includes administering a composition as described herein to a mammal such that expression of the target LECT2 gene is increased by e.g., at least 10% compared to an untreated animal. In some embodiments, the activation of LECT2 occurs over an extended duration, e.g., at least two, three, four days or more, e.g., one week, two weeks, three weeks, four weeks, or more. Without wishing to be bound by theory, an iRNA can activate LECT2 expression by stabilizing the LECT2 mRNA transcript, interacting with a promoter in the genome, and/or inhibiting an inhibitor of LECT2 expression.

The iRNAs and antisense polynucleotide agents useful for the methods and compositions described herein specifically target RNAs (primary or processed) of a LECT2 gene. Compositions and methods for inhibiting the expression of a LECT2 gene using iRNAs or antisense polynucleotide agents can be prepared and performed as described elsewhere herein.

In one embodiment, the method includes administering a composition containing an iRNA or antisense polynucleotide agent, where the iRNA or antisense polynucleotide agent includes a nucleotide sequence that is complementary to at least a part of an RNA transcript of the LECT2 gene of the subject, e.g., the mammal, e.g., the human, to be treated. The composition may be administered by any appropriate means known in the art including, but not limited to oral, intraperitoneal, or parenteral routes, including intracranial (e.g., intraventricular, intraparenchymal and intrathecal), intravenous, intramuscular, subcutaneous, transdermal, airway (aerosol), nasal, rectal, and topical (including buccal and sublingual) administration.

In certain embodiments, the composition is administered by intravenous infusion or injection. In some such embodiments, the composition comprises a lipid formulated siRNA (e.g., an LNP formulation, such as an LNP11 formulation) for intravenous infusion.

In other embodiments, the composition is administered subcutaneously. In some such embodiments, the composition comprises an iRNA conjugated to a GalNAc ligand. In some such embodiments, the ligand targets the iRNA to the liver (e.g., to hepatocytes).

Unless otherwise defined, all technical and scientific terms used herein have the same meaning as commonly understood by one of ordinary skill in the art to which this invention belongs. Although methods and materials similar or equivalent to those described herein can be used in the practice or testing of the iRNAs and methods featured in the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1. Detection of Lect2 mRNA as First Line Screen for ALECT2 Diagnosis

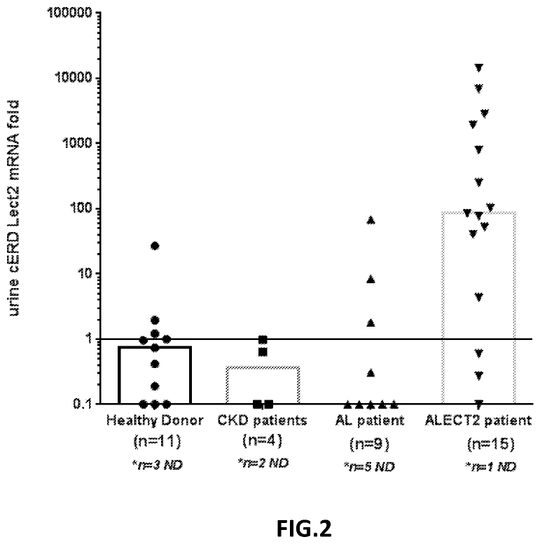

Urine samples were acquired from 16 ALECT2 patients, 11 healthy volunteers, 4 chronic kidney disease (CKD) patients and 9 amyloid light-chain (AL) amyloidosis patients. Using the method described below, the mRNA levels of Lect2 (normalized with respective to Gapdh mRNA levels) of these four groups were compared (FIG. 2). In healthy volunteers, CKD patients, and AL patients, urine Lect2 mRNA level was either undetectable or very low (absolute average Ct value >36). In comparison, Lect2 mRNA levels were up-regulated in a significant number of ALECT2 patients (absolute Ct value ranges from 24-34.)

This assay can serve as a first line screen of non-invasive diagnosis of ALECT2. In some embodiments, this screen can be followed up with established diagnosis such as kidney biopsy.

### Method

RNA Extraction.

0.2-micron filtered urine samples were lyophilized over a 2- to 3-day period using a Labconco 12-L FreeZone Freeze Dry Console (Kansas City, Mo.). Lyophilized samples were resuspended in 8 ml of Qiagen nuclease-free water (Valencia, Calif.) and subjected to differential centrifugation. Samples were first centrifuged at 3,000×g for 10 minutes. The resulting supernatants were centrifuged at 17,000×g for 20 minutes. Lithium chloride (Life Technologies) was added to supernatant urine samples at a final concentration of 1M. Following 1 hour incubation on ice, the samples were ultracentrifuged at 200,000×g for 120 minutes (Beckman Coulter, Brea, Calif.), and supernatants were decanted. One milliliter of trizol (Life Technologies) was added to the pellets, and the samples were vortexed for 60 seconds. Samples were transferred to 1.5 ml microcentrifuge tubes (Life Technologies), and 0.2 ml chloroform (Sigma Aldrich) was added to each sample and mixed thoroughly by inversion. Samples were centrifuged at 16,000×g at 4° C. for 20 minutes. The upper aqueous phase was transferred to a fresh 1.5 ml ultracentrifuge tube and precipitated with an equal volume of isopropanol, 1 μl of GenElute Linear Polyacrylamide (Sigma Aldrich), and 1/10th volume of 3 M sodium acetate, pH 5.5 or less. Samples were centrifuged at 16,000×g at 4° C. for 10 minutes. The resulting RNA pellet was washed twice with ice cold 70% ethanol, air-dried, and resuspended in 20 μl RNase-free water (Life Technologies). 10 μl was used for cDNA synthesis (High Capacity cDNA synthesis kit, Life Technologies). 2 μl of cDNA was used for singleplex qPCR Reactions with SYBR Green buffer. The comparative Ct method was used to quantify fold change relative to one of the healthy volunteer urine sample. The LECT2 mRNA levels were normalized to those of GAPDH. The following primers were used to detect human LECT2 and GAPDH:

LECT2:
(SEQ ID NO: 685)
Fwd-GTGCTGGCAAGTCTTCCAATGAG;
(SEQ ID NO: 686)
Rev-CCAGTGAATGGTGCGTACACAG;
GAPDH:
(SEQ ID NO: 687)
Fwd-GTCTCCTCTGACTTCAACAGCG;
(SEQ ID NO: 688)
Rev-ACCACCCTGTTGCTGTAGCCAA.

### INCORPORATION BY REFERENCE

All publications, patents, and Accession numbers mentioned herein are hereby incorporated by reference in their entirety as if each individual publication or patent was specifically and individually indicated to be incorporated by reference.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A method of determining an activity or expression of a LECT2 gene in a subject, comprising:
i) acquiring a bodily fluid sample from the subject; and
ii) detecting the level of an LECT2 mRNA in the sample;
wherein an increase in the level of the LECT2 mRNA, as compared to a reference LECT2 mRNA level, is indicative of an increase in the activity or expression of the LECT2 gene at a site distal from the sample,
thereby determining the activity or expression of the LECT2 gene in the subject.

2. The method of claim 1, wherein the increase in the level of the LECT2 mRNA in the sample is indicative of the increase in the activity or expression of the LECT2 gene in a cell expressing the LECT2 mRNA.

3. The method of claim 1 or 2, wherein the increase in the level of the LECT2 mRNA in a urine or blood sample is indicative of the increase in the activity or expression of the LECT2 gene in a liver cell.

4. The method of any of claims 1-3, wherein the increase in the activity or expression of the LECT2 gene is indicative of having, or an increased risk of having, a LECT2-associated disorder in the subject.

5. The method of any of claims 1-4, further comprising administering to the subject a double-stranded ribonucleic acid (dsRNA) or antisense polynucleotide agent that inhibits expression of the LECT2 mRNA.

6. A method of evaluating a subject for a LECT2-associated disorder, comprising:
acquiring knowledge of the level of an LECT2 mRNA in a bodily fluid sample from the subject,
wherein an increase in the level of the LECT2 mRNA in the sample, as compared to a reference LECT2 mRNA level, is indicative of having, or an increased risk of having, the LECT2-associated disorder in the subject,
thereby evaluating the subject for the LECT2-associated disorder.

7. The method of claim 6, wherein acquiring knowledge of the level of the LECT2 mRNA comprises detecting the level of the LECT2 mRNA in the sample.

8. The method of claim 6 or 7, wherein the increase in the level of the LECT2 mRNA in the sample is indicative of an increase in an activity or expression of a LECT2 gene at a site distal from the sample.

9. The method of any of claims 6-8, wherein the increase in the level of the LECT2 mRNA in a urine or blood sample is indicative of the increase in the activity or expression of the LECT2 gene in a liver cell.

10. The method of any of claims 6-9, further comprising administering to the subject a dsRNA or antisense polynucleotide agent that inhibits expression of the LECT2 mRNA.

11. A method of identifying a subject for a therapy for a LECT2-associated disorder, the method comprising:
i) acquiring a bodily fluid sample from the subject; and
ii) detecting the level of an LECT2 mRNA in the sample;
wherein an increase in the level of the LECT2 mRNA, as compared to a reference LECT2 mRNA level, is indicative of a subject suitable for the therapy, wherein the therapy comprises a dsRNA or antisense polynucleotide agent inhibits expression of the LECT2 mRNA,
thereby identifying the subject for the therapy for the LECT2-associated disorder.

12. The method of claim 11, wherein the increase in the level of the LECT2 mRNA in the sample is indicative of an increase in an activity or expression of a LECT2 gene at a site distal from the sample.

13. The method of claim 11 or 12, wherein the increase in the level of the LECT2 mRNA in a urine or blood sample is indicative of the increase in the activity or expression of a LECT2 gene in a liver cell.

14. The method of any of claims 11-13, wherein the increase in the activity or expression of the LECT2 gene is indicative of having, or an increased risk of having, the LECT2-associated disorder in the subject.

15. The method of any of claims 11-14, further comprising administering to the subject the dsRNA or the antisense polynucleotide agent that inhibits expression of the LECT2 mRNA.

16. A method of treating a LECT2-related disorder in a subject, comprising:
responsive to the determination of an increase in the level of an LECT2 mRNA in a bodily fluid sample from the subject, as compared to a reference LECT2 mRNA level, administering to the subject a dsRNA or antisense polynucleotide agent that inhibits expression of the LECT2 mRNA,
thereby treating the LECT2-related disorder in the subject.

17. The method of claim 16, further comprising detecting the level of the LECT2 mRNA in the sample.

18. The method of claim 16 or 17, further comprising acquiring the sample from the subject.

19. The method of any of claims 16-18, wherein the increase in the level of the LECT2 mRNA in the sample is indicative of an increase in an activity or expression of a LECT2 gene at a site distal from the sample.

20. The method of any of claims 16-18, wherein the increase in the level of the LECT2 mRNA in a urine or blood sample is indicative of the increase in the activity or expression of a LECT2 gene in a liver cell.

21. A method of reducing an activity or expression of a LECT2 gene, comprising:
i) acquiring knowledge of the level of an LECT2 mRNA encoded by the LECT2 gene in a body fluid sample, wherein an increase in the level of the LECT2 mRNA, as compared to a reference LECT2 mRNA level, is indicative of an increase in the activity or expression of the LECT2 gene in a cell at a site distal to the sample; and
ii) contact the cell with a dsRNA or antisense polynucleotide agent that inhibits expression of the LECT2 mRNA,
thereby reducing the activity or expression of the LECT2 gene.

22. The method of claim 21, further comprising detecting the level of the LECT2 mRNA in the sample.

23. The method of claim 21 or 22, wherein the increase in the level of the LECT2 mRNA in a urine or blood sample is indicative of the increase in the activity or expression of a LECT2 gene in a liver cell.

24. The method of any of claims 21-23, further comprising acquiring the sample from a subject.

25. The method of claim 24, wherein the increase in the activity or expression of the LECT2 gene is indicative of having, or an increased risk of having, a LECT2-associated disorder in the subject.

26. A method of determining an activity of a dsRNA or antisense polynucleotide agent in a subject, the method comprising:
i) acquiring a bodily fluid sample from a subject who has been administered the dsRNA or antisense polynucleotide agent, wherein the dsRNA or antisense polynucleotide agent inhibits expression of an LECT2 mRNA; and
ii) detecting the level of the LECT2 mRNA, or a cleavage product thereof, in the sample,
wherein a decrease in the level of the LECT2 mRNA, or an increase in the level of the cleavage product, as compared to a reference level of the LECT2 mRNA, or the cleavage product thereof, is indicative that the dsRNA or antisense polynucleotide agent is active in the subject.

27. The method of claim 26, wherein the decrease in the level of the LECT2 mRNA, or the increase in the level of the cleavage product, in the sample, is indicative of an inhibition of expression of the LECT2 mRNA at a site distal from the subject.

28. The method of claim 26 or 27, wherein the decrease in the level of the LECT2 mRNA, or the increase in the level of the cleavage product, in a urine or blood sample, is indicative of an inhibition of expression of the LECT2 mRNA in a liver cell.

29. The method of any of claims 26-28, wherein the subject has been administered a therapy for a LECT2-associated disorder comprising the dsRNA or antisense polynucleotide agent.

30. The method of any of claims 26-29, comprising responsive to the determination that the dsRNA or antisense polynucleotide agent is active in the subject, adjusting the dosage of the dsRNA or antisense polynucleotide agent.

31. The method of claim 30, wherein the dose is decreased, the interval between doses is increased, or both.

32. The method of any of claims 26-29, wherein responsive to the determination that the dsRNA or antisense polynucleotide agent is active in the subject, administration of the dsRNA or antisense polynucleotide agent to the subject is continued.

33. The method of claim 26, wherein an increased or unchanged level of the LECT2 mRNA, or a unchanged or decreased level of the cleavage product, is indicative that the dsRNA or antisense polynucleotide agent is inactive in the subject.

34. The method of claim 33, comprising responsive to the determination that the dsRNA or antisense polynucleotide agent is inactive in the subject, adjusting the dosage of the dsRNA or antisense polynucleotide agent.

35. The method of claim 34, wherein the dose is increased, the interval between doses is decreased, or both.

36. The method of claim 33, wherein responsive to the determination that the dsRNA or antisense polynucleotide agent is inactive in the subject, administration of the dsRNA or antisense polynucleotide agent to the subject is discontinued.

37. The method of claim 33 or 36, wherein responsive to the determination that the dsRNA or antisense polynucleotide agent is inactive in the subject, administering to the subject an alternative therapy for a LECT2-associated disorder.

38. A dsRNA or antisense polynucleotide agent for use in treating a LECT2-related disorder in a subject, wherein the dsRNA or antisense polynucleotide agent inhibits expression of a LECT2 mRNA, and wherein the dsRNA or antisense polynucleotide agent is used responsive to the determination of an increase in the level of an LECT2 mRNA in a bodily fluid sample from the subject, as compared to a reference LECT2 mRNA level.

39. The dsRNA or antisense polynucleotide agent for use of claim 38, further comprising detecting the level of the LECT2 mRNA in the sample.

40. The dsRNA or antisense polynucleotide agent for use of claim 38 or 39, further comprising acquiring the sample from the subject.

41. The dsRNA or antisense polynucleotide agent for use of any of claims 38-40, wherein the increase in the level of the LECT2 mRNA in the sample is indicative of an increase in an activity or expression of a LECT2 gene at a site distal from the sample.

42. The dsRNA or antisense polynucleotide agent for use of any of claims 38-40, wherein the increase in the level of the LECT2 mRNA in a urine or blood sample is indicative of the increase in the activity or expression of a LECT2 gene in a liver cell.

43. The method, or the dsRNA or antisense polynucleotide agent for use, of any of claims 1-41, wherein the sample is chosen from a urine sample, a blood sample, a synovial fluid sample, a cerebrospinal fluid (CSF) sample, an amniotic fluid sample, a saliva sample, a breast milk sample, a bronchoalveolar lavage fluid sample, or a malignant ascites sample.

44. The method, or the dsRNA or antisense polynucleotide agent for use, of any of claims 1-41, wherein the sample is a urine sample.

45. The method, or the dsRNA or antisense polynucleotide agent for use, of any of claims 1-41, wherein the sample is a serum sample.

46. The method, or the dsRNA or antisense polynucleotide agent for use, of claim 43, wherein the blood sample is a serum sample or a plasma sample.

47. The method, or the dsRNA or antisense polynucleotide agent for use, of any of claims 1-46, wherein the sample comprises circulating extracellular RNA.

48. The method, or the dsRNA or antisense polynucleotide agent for use, of any of claims 1-47, wherein the sample comprises exosomes.

49. The method, or the dsRNA or antisense polynucleotide agent for use, of any of claims 1-48, wherein the reference level of the LECT2 mRNA is the level of the LECT2 mRNA in a bodily fluid sample from a healthy subject.

50. The method, or the dsRNA or antisense polynucleotide agent for use, of any of claims 1-48, wherein the reference level of the LECT2 mRNA is the level of the LECT2 mRNA in a bodily sample from a subject who does not have a LECT2-related disorder or a symptom of thereof.

51. The method, or the dsRNA or antisense polynucleotide agent for use, of any of claims 1-48, wherein the reference level of the LECT2 mRNA is the level of the LECT2 mRNA in a bodily sample from a subject who has a chronic kidney disease (CKD).

52. The method, or the dsRNA or antisense polynucleotide agent for use, of any of claims 1-48, wherein the reference level of the LECT2 mRNA is the level of the LECT2 mRNA in a bodily sample from a subject who has amyloid light-chain (AL) amyloidosis.

53. The method, or the dsRNA or antisense polynucleotide agent for use, of any of claims 1-48, wherein the reference level of the LECT2 mRNA is the level of the LECT2 mRNA in a bodily sample from the subject prior to administration of a dsRNA or antisense polynucleotide agent.

54. The method, or the dsRNA or antisense polynucleotide agent for use, of any of claims 1-48, wherein the reference level of the LECT2 mRNA is the level of the LECT2 mRNA in a bodily fluid sample from the subject within 1 hour, 2 hours, 6 hours, 12 hours, 24 hours, 48 hours, 72 hours, or 96 hours after administration of a dsRNA or antisense polynucleotide agent.

55. The method, or the dsRNA or antisense polynucleotide agent for use, of any of claims 1-48, wherein the reference level of the LECT2 mRNA is the average level of the LECT2 mRNA in bodily fluid samples from a plurality of subjects.

56. The method, or the dsRNA or antisense polynucleotide agent for use, of any of claims 1-48, wherein the reference level of the cleavage product is the level of the cleavage product in a bodily sample from a subject who has not been administered a dsRNA or antisense polynucleotide agent.

57. The method, or the dsRNA or antisense polynucleotide agent for use, of any of claims 1-48, wherein the reference level of the cleavage product is the level of the cleavage product in a sample from the subject within 1 hour, 2 hours, 6 hours, 12 hours, 24 hours, 48 hours, 72 hours, or 96 hours after administration of a dsRNA or antisense polynucleotide agent.

58. The method, or the dsRNA or antisense polynucleotide agent for use, of any of claims 1-48, wherein the reference level of the cleavage product is the average level of the cleavage product in bodily samples from a plurality of subjects.

59. The method, or the dsRNA or antisense polynucleotide agent for use, of any of claims 1-58, comprises centrifuging the sample prior to detecting the level of the LECT2 mRNA, or the cleavage product thereof.

60. The method, or the dsRNA or antisense polynucleotide agent for use, of any of claims 1-59, wherein the sample is centrifuged at 100,000 g to 300,000 g.

61. The method, or the dsRNA or antisense polynucleotide agent for use, of any of claims 1-60, wherein the sample is centrifuged at about 200,000 g.

62. The method, or the dsRNA or antisense polynucleotide agent for use, of any of claims 1-61, comprising producing a cDNA complementary to the LECT2 mRNA or the cleavage product thereof.

63. The method, or the dsRNA or antisense polynucleotide agent for use, of claim 62, further comprising amplifying the cDNA.

64. The method, or the dsRNA or antisense polynucleotide agent for use, of any of claims 1-63, comprising detecting the level of the LECT2 mRNA, or the cleavage product thereof, using 5′ RACE, hybridization, polymerase chain reaction (PCR), quantitative PCR (qPCR), branched DNA (bDNA), or reverse transcription-PCR (RT-PCR).

65. The method, or the dsRNA or antisense polynucleotide agent for use, of any of claims 1-64, comprising contacting the sample with a reagent for isolating RNA.

66. The method, or the dsRNA or antisense polynucleotide agent for use, of claim 65, wherein the reagent for isolating RNA comprises TRIzol™ reagent, chloroform, phenol/chloroform, or a combination thereof.

67. The method, or the dsRNA or antisense polynucleotide agent for use, of any of claims 1-66, comprising contacting the sample with a reagent for enhancing RNA precipitation.

68. The method, or the dsRNA or antisense polynucleotide agent for use, of claim 67, wherein the reagent for enhancing RNA precipitation comprises glycogen or polyethylene glycol (PEG).

69. The method, or the dsRNA or antisense polynucleotide agent for use, of any of claims 1-68, comprising contacting the sample with an RNase inhibitor.

70. The method, or the dsRNA or antisense polynucleotide agent for use, of claim 69, wherein the RNase inhibitor comprises EDTA.

71. The method, or the dsRNA or antisense polynucleotide agent for use, of any of claims 1-70, comprising contacting the sample with a reagent for increasing RNA yield.

72. The method, or the dsRNA or antisense polynucleotide agent for use, of claim 71, wherein the reagent for increasing RNA yield comprises lithium chloride (LiCl).

73. The method, or the dsRNA or antisense polynucleotide agent for use, of claim 71 or 72, wherein the reagent for increasing RNA yield is used at a final concentration of 1 M.

74. The method, or the dsRNA or antisense polynucleotide agent for use, of any of claims 66-68, wherein the reagent for increasing RNA yield is contacted with the sample prior to precipitation of the RNA.

75. The method, or the dsRNA or antisense polynucleotide agent for use, of claim 65 or 66, comprising contacting the isolated RNA with a primer.

76. The method, or the dsRNA or antisense polynucleotide agent for use, of claim 75, wherein the primer comprises an oligo-dT sequence.

77. The method, or the dsRNA or antisense polynucleotide agent for use, of claim 75, wherein the primer is suitable for PCR.

78. The method, or the dsRNA or antisense polynucleotide agent for use, of claim 75, wherein the primer is suitable for 5′ RACE.

79. The method, or the dsRNA or antisense polynucleotide agent for use, of any of claims 1-78, comprising normalizing the level of the LECT2 mRNA or the cleavage product thereof.

80. The method, or the dsRNA or antisense polynucleotide agent for use, of claim 79, wherein the level of the LECT2 mRNA or the cleavage product thereof is normalized to the level of 18s RNA, GAPDH mRNA, or β-actin mRNA in the same sample.

81. The method, or the dsRNA or antisense polynucleotide agent for use, of claim 79, wherein the level of the LECT2 mRNA or the cleavage product thereof is normalized to the level of an mRNA encoding a liver protein in the same sample.

82. The method, or the dsRNA or antisense polynucleotide agent for use, of claim 81, wherein the liver protein is factor VII, albumin, or alpha antitrypsin (AAT).

83. The method, or the dsRNA or antisense polynucleotide agent for use, of any of claims 1-82, wherein the level of the LECT2 mRNA or the cleavage product thereof is detected without purification (e.g., affinity purification) of exosomes from the sample.

84. The method, or the dsRNA or antisense polynucleotide agent for use, of any of claim 5 or 10-83, wherein the dsRNA or antisense polynucleotide agent targets a LECT2 mRNA expressed in liver, kidney, brain, spinal cord, choroid plexus, peripheral neurons or nerve, muscle, endothelial cells, heart, immune cells, skin, eye, pancreas, lung, stomach, small or large intestines, colon, adrenal gland, tumors, cancer lesions, or spleen.

85. The method, or the dsRNA or antisense polynucleotide agent for use, of any of claim 5 or 10-84, wherein the dsRNA or antisense polynucleotide agent targets a LECT2 mRNA expressed in liver.

86. The method, or the dsRNA or antisense polynucleotide agent for use, of any of claim 5 or 10-85, wherein the dsRNA or antisense polynucleotide agent reduces LECT2 mRNA expression in a cell in the subject.

87. The method, or the dsRNA or antisense polynucleotide agent for use, of claim 86, wherein the cell is a liver cell or a hepatocyte.

88. The method, or the dsRNA or antisense polynucleotide agent for use, of any of claim 5 or 10-87, wherein the dsRNA or antisense polynucleotide agent reduces LECT2 mRNA expression in the subject by at least 20%.

89. The method, or the dsRNA or antisense polynucleotide agent for use, of any of claim 5 or 10-88, wherein the dsRNA or antisense polynucleotide agent reduces LECT2 mRNA expression in the subject by at least 30%.

90. The method, or the dsRNA or antisense polynucleotide agent for use, of any of claim 5 or 10-89, wherein the dsRNA or antisense polynucleotide agent reduces LECT2 mRNA expression in the subject by at least 40%.

91. The method, or the dsRNA or antisense polynucleotide agent for use, of any of claim 5 or 10-90, wherein the dsRNA or antisense polynucleotide agent reduces LECT2 mRNA expression in the subject by at least 50%.

92. The method, or the dsRNA or antisense polynucleotide agent for use, of any of claim 5 or 10-91, wherein the dsRNA or antisense polynucleotide agent reduces the level of LECT2 mRNA in urine or blood, by at least 10%, 20%, 30%, 40%, or 50%, within 1, 2, 3, 7, 14, 21, or 28 days after administration of the dsRNA or antisense polynucleotide agent.

93. The method, or the dsRNA or antisense polynucleotide agent for use, of any of claim 5 or 10-92, wherein the dsRNA or antisense polynucleotide agent reduces the level of LECT2 mRNA in a liver, by at least 10%, 20%, 30%, 40%, or 50%, within 1, 2, 3, 7, 14, 21, or 28 days after administration of the dsRNA or antisense polynucleotide agent.

94. The method, or the dsRNA or antisense polynucleotide agent for use, of any of claim 5 or 10-93, wherein the dsRNA comprises a sense strand that is 15-30 base pairs in length and an antisense strand that is 15-30 base pairs in length, wherein the antisense strand is complementary to at least 15 contiguous nucleotides of SEQ ID NO: 1 or a nucleotide sequence having an A to G substitution at nucleotide position 373 of SEQ ID NO: 1.

95. The method, or the dsRNA or antisense polynucleotide agent for use, of any of claim 5 or 10-94, wherein the dsRNA comprises a sense strand and an antisense strand, the antisense strand comprising a region of complementarity to a LECT2 RNA transcript, wherein the antisense strand comprises at least 15 contiguous nucleotides differing by no more than 3 nucleotides from one of the antisense sequences listed in Tables 2 and 3.

96. The method, or the dsRNA or antisense polynucleotide agent for use, of any of claim 5 or 10-95, wherein the dsRNA comprises at least one modified nucleotide.

97. The method, or the dsRNA or antisense polynucleotide agent for use, of any of claim 5 or 10-96, wherein the dsRNA comprises a duplex region that is 15-30 nucleotide pairs in length.

98. The method, or the dsRNA or antisense polynucleotide agent for use, of claim 97, wherein the duplex region is 17-23 nucleotide pairs in length.

99. The method, or the dsRNA or antisense polynucleotide agent for use, of claim 97 or 98, wherein the duplex region is 19-21 nucleotide pairs in length.

100. The method, or the dsRNA or antisense polynucleotide agent for use, of any of claims 97-99, wherein the duplex region is 21-23 nucleotide pairs in length.

101. The method, or the dsRNA or antisense polynucleotide agent for use, of any of claim 5 or 10-100, wherein the dsRNA comprises a region of complementarity that is at least 17 nucleotides in length.

102. The method, or the dsRNA or antisense polynucleotide agent for use, of claim 101, wherein the region of complementarity is between 19 and 21 nucleotides in length.

103. The method, or the dsRNA or antisense polynucleotide agent for use, of claim 101 or 102, wherein the region of complementarity is 19 nucleotides in length.

104. The method, or the dsRNA or antisense polynucleotide agent for use, of any of claim 5 or 10-103, wherein at least one strand of the dsRNA comprises a 3′ overhang of at least 1 nucleotide.

105. The method, or the dsRNA or antisense polynucleotide agent for use, of any of claim 5 or 10-104, wherein at least one strand of the dsRNA comprises a 3′ overhang of at least 2 nucleotides.

106. The method, or the dsRNA or antisense polynucleotide agent for use, of any of claims 95-105, wherein the at least one modified nucleotide is chosen from a 2′-O-methyl modified nucleotide, a nucleotide comprising a 5′-phosphorothioate group, or a terminal nucleotide linked to a cholesteryl derivative or dodecanoic acid bisdecylamide group.

107. The method, or the dsRNA or antisense polynucleotide agent for use, of any of claims 95-106, wherein the at least one modified nucleotide is chosen from a 2′-deoxy-2′-fluoro modified nucleotide, a 2′-deoxy-modified nucleotide, a locked nucleic acid (LNA), an acyclic nucleotide, an abasic nucleotide, 2′-amino-modified nucleotide, 2′-alkyl-modified nucleotide, morpholino nucleotide, a phosphoramidate, or a non-natural base comprising nucleotide.

108. The method, or the dsRNA or antisense polynucleotide agent for use, of any of claims 95-107, wherein the at least one modified nucleotide comprises a modification selected from the group consisting of locked nucleic acid (LNA), an acyclic nucleotide, hexitol or hexose nucleic acid (HNA), cyclohexene nucleic acid (CeNA), 2′-methoxyethyl, 2′-O-alkyl, 2′-O-allyl, 2′-C-allyl, 2′-fluoro, 2′-O-methyl, 2′-deoxy, 2′-hydroxyl, and combinations thereof.

109. The method, or the dsRNA or antisense polynucleotide agent for use, of any of claims 95-108, wherein the at least one modified nucleotide comprises 2′-O-methyl, 2′-fluoro, or both.

110. The method, or the dsRNA or antisense polynucleotide agent for use, of any of claims 95-109, wherein the dsRNA comprises a sense strand that is conjugated to at least one ligand.

111. The method, or the dsRNA or antisense polynucleotide agent for use, of claim 110, wherein the ligand is attached to the 3′ end of the sense strand.

112. The method, or the dsRNA or antisense polynucleotide agent for use, of any of claim 110 or 111, wherein the ligand comprises a carbohydrate.

113. The method, or the dsRNA or antisense polynucleotide agent for use, of any of claims 110-112, wherein the ligand is a GalNAc ligand.

114. The method, or the dsRNA or antisense polynucleotide agent for use, of any of claims 110-113, wherein the ligand is

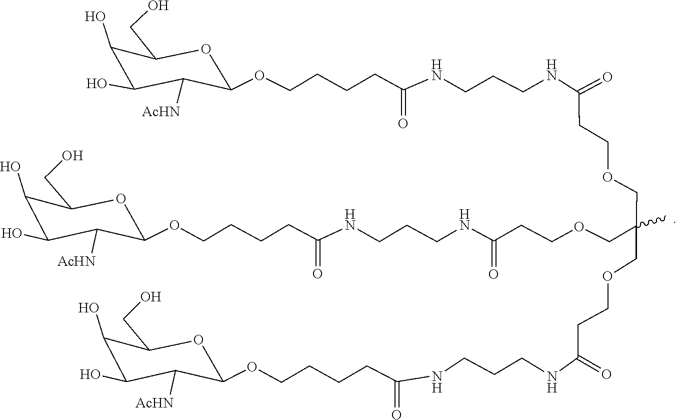 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

115. The method, or the dsRNA or antisense polynucleotide agent for use, of any of claims 110-114, wherein the ligand is attached via a linker.

116. The method, or the dsRNA or antisense polynucleotide agent for use, of claim 115, wherein the linker is a bivalent or trivalent branched linker.

117. The method, or the dsRNA or antisense polynucleotide agent for use, of claim 115 or 116, wherein the ligand and linker are as shown in Formula XXIV:

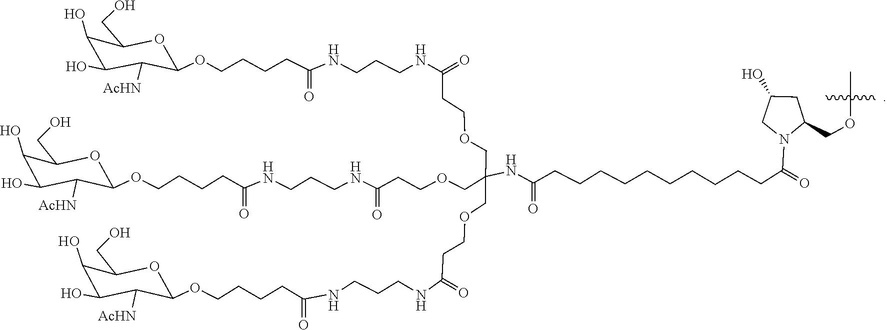 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

118. The method, or the dsRNA or antisense polynucleotide agent for use, of any of claims 110-117, wherein the ligand targets the dsRNA to hepatocytes.

119. The method, or the dsRNA or antisense polynucleotide agent for use, of any of claim 5 or 10-118, wherein the dsRNA comprises a region of complementarity that comprises an antisense sequence selected from the antisense sequences disclosed in Tables 2 and 3.

120. The method, or the dsRNA or antisense polynucleotide agent for use, of any of claim 5 or 10-119, wherein the dsRNA comprises a sense strand comprising a sense sequence selected from the sense sequences disclosed in Tables 2 and 3, and an antisense strand comprising an antisense sequence selected from the antisense sequences disclosed in Tables 2 and 3.

121. The method, or the dsRNA or antisense polynucleotide agent for use, of any of claim 5 or 10-120, wherein the dsRNA comprises a region of complementarity that consists of an antisense sequence selected from the antisense sequences disclosed in Tables 2 and 3.

122. The method, or the dsRNA or antisense polynucleotide agent for use, of any of claim 5 or 10-121, wherein the dsRNA comprises a sense strand consisting of a sense sequence selected from the sense sequences disclosed in Tables 2 and 3, and an antisense strand consisting of an antisense sequence selected from the antisense sequences disclosed in Tables 2 and 3.

123. The method, or the dsRNA or antisense polynucleotide agent for use, of any of claim 5 or 10-93, wherein the antisense polynucleotide agent comprises about 4 to about 50 contiguous nucleotides, wherein at least one of the contiguous nucleotides is a modified nucleotide, and wherein the nucleotide sequence of the agent is about 80% complementary over its entire length to the equivalent region of the nucleotide sequence of SEQ ID NO: 1.

124. The method, or the dsRNA or antisense polynucleotide agent for use, of claim 123, wherein the equivalent region is one of the target regions of SEQ ID NO: 1 provided in Table 3.

125. The method, or the dsRNA or antisense polynucleotide agent for use, of any of claim 5, 10-93, or 124, wherein the antisense polynucleotide agent comprises at least 8 contiguous nucleotides differing by no more than 3 nucleotides from any one of the nucleotide sequences listed in Table 3.

126. The method, or the dsRNA or antisense polynucleotide agent for use, of any of claim 5, 10-93, or 123-125, wherein the antisense polynucleotide agent is 10 to 40 nucleotides in length.

127. The method, or the dsRNA or antisense polynucleotide agent for use, of any of claim 5, 10-93, or 123-126, wherein the antisense polynucleotide agent is 10 to 30 nucleotides in length.

128. The method, or the dsRNA or antisense polynucleotide agent for use, of any of claim 5, 10-93, or 123-127, wherein the antisense polynucleotide agent is 18 to 30 nucleotides in length.

129. The method, or the dsRNA or antisense polynucleotide agent for use, of any of claim 5, 10-93, or 123-128, wherein the antisense polynucleotide agent is 10 to 24 nucleotides in length.

130. The method, or the dsRNA or antisense polynucleotide agent for use, of any of claim 5, 10-93, or 123-129, wherein the antisense polynucleotide agent is 18 to 24 nucleotides in length.

131. The method, or the dsRNA or antisense polynucleotide agent for use, of any of claim 5, 10-93, or 123-130, wherein the antisense polynucleotide agent is 14 or 20 nucleotides in length.

132. The method, or the dsRNA or antisense polynucleotide agent for use, of any of claim 5, 10-93, or 123-131, wherein substantially all of the nucleotides of the antisense polynucleotide agent are modified nucleotides.

133. The method, or the dsRNA or antisense polynucleotide agent for use, of any of claim 5, 10-93, or 123-132, wherein all of the nucleotides of the antisense polynucleotide agent are modified nucleotides.

134. The method, or the dsRNA or antisense polynucleotide agent for use, of claim 132 or 133, wherein the modified nucleotide comprises a modified sugar moiety selected from the group consisting of: a 2′-O-methoxyethyl modified sugar moiety, a 2′-methoxy modified sugar moiety, a 2′-O-alkyl modified sugar moiety, and a bicyclic sugar moiety.

135. The method, or the dsRNA or antisense polynucleotide agent for use, of claim 134, wherein the bicyclic sugar moiety has a (—CH2-)n group forming a bridge between the 2′ oxygen and the 4′ carbon atoms of the sugar ring, wherein n is 1 or 2.

136. The method, or the dsRNA or antisense polynucleotide agent for use, of any of claims 132-135, wherein the modified nucleotide is a 5-methylcytosine.

137. The method, or the dsRNA or antisense polynucleotide agent for use, of any of claims 132-136, wherein the modified nucleotide comprises a modified internucleoside linkage.

138. The method, or the dsRNA or antisense polynucleotide agent for use, of claim 137, wherein the modified internucleoside linkage is a phosphorothioate internucleoside linkage.

139. The method, or the dsRNA or antisense polynucleotide agent for use, of any of claim 5, 10-93, or 123-138, wherein the antisense polynucleotide agent comprises a plurality of 2′-deoxynucleotides flanked on each side by at least one nucleotide having a modified sugar moiety.

140. The method, or the dsRNA or antisense polynucleotide agent for use, of claim 139, wherein the modified sugar moiety is chosen from a 2′-O-methoxyethyl modified sugar moiety, a 2′-methoxy modified sugar moiety, a 2′-O-alkyl modified sugar moiety, or a bicyclic sugar moiety.

141. The method, or the dsRNA or antisense polynucleotide agent for use, of any of claim 5, 10-93, or 123-140, wherein the antisense polynucleotide agent is a gapmer comprising a gap segment comprised of linked 2′-deoxynucleotides positioned between a 5′ and a 3′ wing segment.

142. The method, or the dsRNA or antisense polynucleotide agent for use, of claim 141, wherein the 5′-wing segment is 1 to 6 nucleotides in length.

143. The method, or the dsRNA or antisense polynucleotide agent for use, of any of claim 141 or 142, wherein the 3′-wing segment is 1 to 6 nucleotides in length.

144. The method, or the dsRNA or antisense polynucleotide agent for use, of any of claims 141-143, wherein the gap segment is 5 to 14 nucleotides in length.

145. The method, or the dsRNA or antisense polynucleotide agent for use, of any of claims 141-144, wherein the 5′-wing segment is 2 nucleotides in length.

146. The method, or the dsRNA or antisense polynucleotide agent for use, of any of claims 141-145, wherein the 3′-wing segment is 2 nucleotides in length.

147. The method, or the dsRNA or antisense polynucleotide agent for use, of any of claims 141-146, wherein the 5′-wing segment is 3 nucleotides in length.

148. The method, or the dsRNA or antisense polynucleotide agent for use, of any of claims 141-147, wherein the 3′-wing segment is 3 nucleotides in length.

149. The method, or the dsRNA or antisense polynucleotide agent for use, of any of claims 141-148, wherein the 5′-wing segment is 4 nucleotides in length.

150. The method, or the dsRNA or antisense polynucleotide agent for use, of any of claims 141-149, wherein the 3′-wing segment is 4 nucleotides in length.

151. The method, or the dsRNA or antisense polynucleotide agent for use, of any of claims 141-150, wherein the 5′-wing segment is 5 nucleotides in length.

152. The method, or the dsRNA or antisense polynucleotide agent for use, of any of claims 141-151, wherein the 3′-wing segment is 5 nucleotides in length.

153. The method, or the dsRNA or antisense polynucleotide agent for use, of any of claims 141-152, wherein gap segment is 10 nucleotides in length.

154. The method, or the dsRNA or antisense polynucleotide agent for use, of any of claim 5, 10-93, or 123-153, wherein the antisense polynucleotide agent comprises:
a gap segment consisting of linked deoxynucleotides;
a 5′-wing segment consisting of linked nucleotides;
a 3′-wing segment consisting of linked nucleotides;
wherein the gap segment is positioned between the 5′-wing segment and the 3′-wing segment and wherein each nucleotide of each wing segment comprises a modified sugar.

155. The method, or the dsRNA or antisense polynucleotide agent for use, of claim 154, wherein the gap segment is ten 2′-deoxynucleotides in length and each of the wing segments is five nucleotides in length.

156. The method, or the dsRNA or antisense polynucleotide agent for use, of claim 154, wherein the gap segment is ten 2′-deoxynucleotides in length and each of the wing segments is four nucleotides in length.

157. The method, or the dsRNA or antisense polynucleotide agent for use, of claim 154, wherein the gap segment is ten 2′-deoxynucleotides in length and each of the wing segments is three nucleotides in length.

158. The method, or the dsRNA or antisense polynucleotide agent for use, of claim 154, wherein the gap segment is ten 2′-deoxynucleotides in length and each of the wing segments is two nucleotides in length.

159. The method, or the dsRNA or antisense polynucleotide agent for use, of any of claims 154-158, wherein the modified sugar moiety is selected from the group consisting of a 2′-O-methoxyethyl modified sugar moiety, a 2′-methoxy modified sugar moiety, a 2′-O-alkyl modified sugar moiety, and a bicyclic sugar moiety.

160. The method, or the dsRNA or antisense polynucleotide agent for use, of any of claim 5, 10-93, or 123-159, wherein the antisense polynucleotide agent further comprises a ligand.

161. The method, or the dsRNA or antisense polynucleotide agent for use, of claim 160, wherein the antisense polynucleotide agent is conjugated to the ligand at the 3′-terminus.

162. The method, or the dsRNA or antisense polynucleotide agent for use, of any of claim 160 or 161, wherein the ligand is an N-acetylgalactosamine (GalNAc) derivative.

163. The method, or the dsRNA or antisense polynucleotide agent for use, of any of claims 160-162, wherein the ligand is

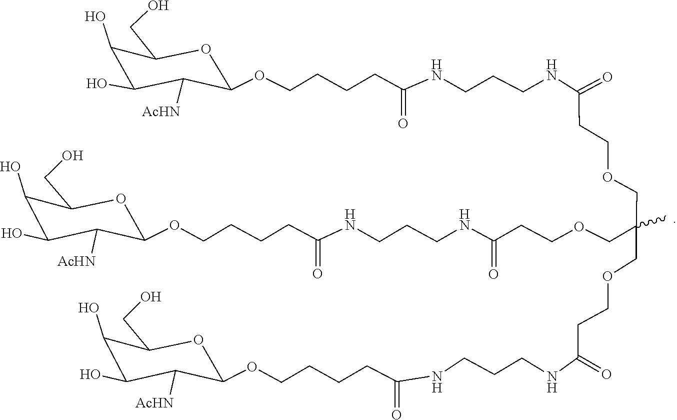 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

164. The method, or the dsRNA or antisense polynucleotide agent for use, of any of claim 5 or 10-163, wherein the dsRNA or antisense polynucleotide agent is administered to the subject using direct injection or infusion, intravenous, intraperitoneal, subcutaneous, intramuscular, inhalation, topical, intracranial, intracerebroventricular, epidural, intrathecal, intraarterial, intravitrial, intradermal, oral, or intracardiac delivery.

165. The method, or the dsRNA or antisense polynucleotide agent for use, of any of claim 5 or 10-164, wherein the dsRNA or antisense polynucleotide agent is administered to the subject intravenously.

166. The method, or the dsRNA or antisense polynucleotide agent for use, of any of claim 5 or 10-164, wherein the dsRNA or antisense polynucleotide agent is administered to the subject subcutaneously.

167. The method, or the dsRNA or antisense polynucleotide agent for use, of any of claim 5 or 10-166, wherein the dsRNA or antisense polynucleotide agent is administered to the subject in an unbuffered solution.

168. The method, or the dsRNA or antisense polynucleotide agent for use, of claim 167, wherein the unbuffered solution is saline or water.

169. The method, or the dsRNA or antisense polynucleotide agent for use, of any of claim 5 or 10-168, wherein the dsRNA or antisense polynucleotide agent is administered to the subject with a buffer solution.

170. The method, or the dsRNA or antisense polynucleotide agent for use, of claim 169, wherein the buffer solution comprises acetate, citrate, prolamine, carbonate, phosphate or any combination thereof.

171. The method, or the dsRNA or antisense polynucleotide agent for use, of claim 169 or 170, wherein the buffer solution is phosphate buffered saline (PBS).

172. The method, or the dsRNA or antisense polynucleotide agent for use, of any of claim 5 or 10-171, wherein the dsRNA or antisense polynucleotide agent is administered to the subject in a pharmaceutical composition comprising a lipid formulation.

173. The method, or the dsRNA or antisense polynucleotide agent for use, of claim 172, wherein the lipid formulation is an LNP formulation.

174. The method, or the dsRNA or antisense polynucleotide agent for use, of claim 172 or 173, wherein the lipid formulation is a LNP11 formulation.

175. The method, or the dsRNA or antisense polynucleotide agent for use, of any of claim 5, 10-165, or 167-174, wherein the dsRNA or antisense polynucleotide agent is administered to the subject intravenously in a pharmaceutical composition comprising a lipid formulation.

176. The method, or the dsRNA or antisense polynucleotide agent for use, of any of claim 5 or 10-175, wherein dsRNA or antisense polynucleotide agent is targeted to a liver cell or a hepatocyte.

177. The method, or the dsRNA or antisense polynucleotide agent for use, of any of claim 5 or 10-176, wherein the dsRNA or antisense polynucleotide agent is conjugated to a ligand chosen from a carbohydrate ligand or a GalNAc ligand.

178. The method, or the dsRNA or antisense polynucleotide agent for use, of any of claim 5 or 10-177, wherein the dsRNA or antisense polynucleotide agent is administered according to a dosing regimen.

179. The method, or the dsRNA or antisense polynucleotide agent for use, of any of claim 5 or 10-178, wherein the dosing regimen is weekly, biweekly, or monthly.

180. The method, or the dsRNA or antisense polynucleotide agent for use, of any of claim 5 or 10-177, wherein the dsRNA or antisense polynucleotide agent is administered to the subject once a week.

181. The method, or the dsRNA or antisense polynucleotide agent for use, of any of claim 5 or 10-177, wherein the dsRNA or antisense polynucleotide agent is administered to the subject twice a week.

182. The method, or the dsRNA or antisense polynucleotide agent for use, of any of claims 5 or 10-177, wherein the dsRNA or antisense polynucleotide agent is administered to the subject twice a month.

183. The method, or the dsRNA or antisense polynucleotide agent for use, of any of claim 5 or 10-182, wherein the dsRNA or antisense polynucleotide agent is administered at a dose of about 0.01 mg/kg to about 100 mg/kg bodyweight of the subject.

184. The method, or the dsRNA or antisense polynucleotide agent for use, of any of claim 5 or 10-183, wherein the dsRNA or antisense polynucleotide agent is administered at a dose of about 0.05 mg/kg to about 50 mg/kg bodyweight of the subject.

185. The method, or the dsRNA or antisense polynucleotide agent for use, of any of claim 5 or 10-184, wherein the dsRNA or antisense polynucleotide agent is administered at a dose of about 0.01 mg/kg to about 5 mg/kg bodyweight of the subject.

186. The method, or the dsRNA or antisense polynucleotide agent for use, of any of claim 5 or 10-185, wherein the dsRNA or antisense polynucleotide agent is administered at a dose of about 0.1 mg/kg to about 0.5 mg/kg bodyweight of the subject.

187. The method, or the dsRNA or antisense polynucleotide agent for use, of any of claim 5 or 10-186, wherein the dsRNA or antisense polynucleotide agent is administered at a dose of about 0.5 mg/kg to about 10 mg/kg bodyweight of the subject.

188. The method, or the dsRNA or antisense polynucleotide agent for use, of any of claim 5 or 10-187, wherein the dsRNA or antisense polynucleotide agent is administered at a dose of about 1 mg/kg to about 10 mg/kg bodyweight of the subject.

189. The method, or the dsRNA or antisense polynucleotide agent for use, of any of claim 4-20, 25, 29-32, or 37-188, wherein the disorder is amyloidosis.

190. The method, or the dsRNA or antisense polynucleotide agent for use, of claim 189, wherein the amyloidosis is a LECT2 amyloidosis (ALECT2).

191. The method, or the dsRNA or antisense polynucleotide agent for use, of any of claim 4-20, 25, 29-32, or 37-188, wherein the disorder is rheumatoid arthritis.

192. The method, or the dsRNA or antisense polynucleotide agent for use, of any of claim 4-20, 25, 29-32, or 37-188, wherein the disorder is an acute liver injury.

193. The method, or the dsRNA or antisense polynucleotide agent for use, of any of claim 5 or 10-192, further comprising administering a second therapy, to the subject.

194. The method, or the dsRNA or antisense polynucleotide agent for use, of claim 193, wherein the second therapy is a therapy that supports kidney function or a therapy that supports liver function.

195. The method, or the dsRNA or antisense polynucleotide agent for use, of claim 194, wherein the therapy that supports kidney function is selected from dialysis, a diuretic, an angiotensin converting enzyme (ACE) inhibitor, or an angiotensin receptor blocker (ARB).

196. The method, or the dsRNA or antisense polynucleotide agent for use, of claim 193, wherein the second therapy is removal of all or part of the organs affected by the amyloidosis.


SEQUENCE LISTING (688 sequences)

SEQ ID NO: 1 - DNA, 1077 bp, Homo sapiens
aaatcaaatagctatccatggaatattagaacttgacttgctccatcctcttaaactttttgtgtctcacactaaagaaatgagagatgcagaattctaaggctaaatagctaggaagtattcattcaaacttgaatattcttcaaagagagtgtgggggcaactctaatcagaggaagaaactaaaggaagtaaaaccagatgttttccaccaaagccctccttttggctggtctgatttctaccgcactggcagggccatgggctaatatatgtgctggcaagtcttccaatgagatccggacgtgtgaccgccatggctgtggacagtactctgctcaaagaagtcagaggcctcaccagggtgtggacatcttgtgctctgctggatctactgtgtacgcaccattcactggaatgattgtgggccaggagaaaccttatcaaaacaagaatgctatcaataatggtgttcgaatatctggaagaggtttttgtgtcaaaatgttctacattaagccaattaagtataaaggtcctattaagaagggagaaaaacttggaactctattgcccttgcagaaagtttatcctggcatacaatcgcatgtgcacattgaaaactgtgactcgagtgaccctactgcatacctgtaaatcgaaggccaatggtcagatcttcaaaataaaaagtcatcttaaaaacctggatgcatacccttctcttcaagaaatttgtgttcacaaaggaaaaatgcatgaagggatggataccccattttccatgacatgattattacacattgcatgcctgtatcaaaacatctcacgtacctcataaacatatacacctatgtacccacaaaaattttttaattaaaaaaaggaaatttgagtttaaatagaaacatgataaatgcaagaaagaaaacattttgattttaactcattgtcactctgatgttcatgtgaactggttgcttcgggctctttgatctgtcacctatggaatctgagtggttttattttttagatttctcagtcccaaagatctaagataaataaacaagagaactt

SEQ ID NO: 2-12 - skipped (11 entries)

SEQ ID NO: 13 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
uucuaatattccatggauag

SEQ ID NO: 14 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
gagcaagtcaagttcuaaua

SEQ ID NO: 15 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
aagaggatggagcaagucaa

SEQ ID NO: 16 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
acaaaaagtttaagaggaug

SEQ ID NO: 17 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
uuuagtgtgagacacaaaaa

SEQ ID NO: 18 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
ucucatttctttagtgugag

SEQ ID NO: 19 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
gaauuctgcatctctcauuu

SEQ ID NO: 20 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
uauuuagccttagaauucug

SEQ ID NO: 21 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
acuucctagctatttagccu

SEQ ID NO: 22 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
guuugaatgaatactuccua

SEQ ID NO: 23 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
aauautcaagtttgaaugaa

SEQ ID NO: 24 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
ucucuttgaagaatauucaa

SEQ ID NO: 25 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
guugcccccacactcucuuu

SEQ ID NO: 26 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
cugautagagttgcccccac

SEQ ID NO: 27 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
uaguutcttcctctgauuag

SEQ ID NO: 28 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
uuuacttcctttagtuucuu

SEQ ID NO: 29 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
aacauctggttttacuuccu

SEQ ID NO: 30 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
ggcuutggtggaaaacaucu

SEQ ID NO: 31 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
gccaaaaggagggctuuggu

SEQ ID NO: 32 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
aaucagaccagccaaaagga

SEQ ID NO: 33 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
gugcggtagaaatcagacca

SEQ ID NO: 34 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
auggccctgccagtgcggua

SEQ ID NO: 35 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
uauautagcccatggcccug

SEQ ID NO: 36 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
cuugccagcacatatauuag

SEQ ID NO: 37 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
cauuggaagacttgccagca

SEQ ID NO: 38 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
acguccggatctcatuggaa

SEQ ID NO: 39 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
auggcggtcacacgtccgga

SEQ ID NO: 40 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
guccacagccatggcgguca

SEQ ID NO: 41 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
gagcagagtactgtccacag

SEQ ID NO: 42 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
ugacutctttgagcagagua

SEQ ID NO: 43 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
gugaggcctctgactucuuu

SEQ ID NO: 44 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
uccacaccctggtgaggccu

SEQ ID NO: 45 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
gagcacaagatgtccacacc

SEQ ID NO: 46 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
agauccagcagagcacaaga

SEQ ID NO: 47 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
ugcguacacagtagauccag

SEQ ID NO: 48 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
cagugaatggtgcgtacaca

SEQ ID NO: 49 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
cacaatcattccagtgaaug

SEQ ID NO: 50 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
uucucctggcccacaaucau

SEQ ID NO: 51 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
guuuugataaggtttcuccu

SEQ ID NO: 52 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
uagcattcttgttttgauaa

SEQ ID NO: 53 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
ccauuattgatagcauucuu

SEQ ID NO: 54 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
gauautcgaacaccauuauu

SEQ ID NO: 55 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
aaacctcttccagatauucg

SEQ ID NO: 56 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
uuuugacacaaaaaccucuu

SEQ ID NO: 57 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
uguagaacattttgacacaa

SEQ ID NO: 58 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
uuaautggcttaatguagaa

SEQ ID NO: 59 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
ccuuuatacttaattggcuu

SEQ ID NO: 60 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
uucuuaataggacctuuaua

SEQ ID NO: 61 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
aguuuttctcccttcuuaau

SEQ ID NO: 62 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
auagagttccaagttuuucu

SEQ ID NO: 63 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
gcaagggcaatagaguucca

SEQ ID NO: 64 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
auaaactttctgcaagggca

SEQ ID NO: 65 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
auuguatgccaggataaacu

SEQ ID NO: 66 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
gcacatgcgattgtaugcca

SEQ ID NO: 67 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
aguuutcaatgtgcacaugc

SEQ ID NO: 68 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
acucgagtcacagttuucaa

SEQ ID NO: 69 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
caguagggtcactcgaguca

SEQ ID NO: 70 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
uuuacaggtatgcaguaggg

SEQ ID NO: 71 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
uggccttcgatttacaggua

SEQ ID NO: 72 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
aagauctgaccattggccuu

SEQ ID NO: 73 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
uuuuuattttgaagaucuga

SEQ ID NO: 74 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
uuaagatgactttttauuuu

SEQ ID NO: 75 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
cauccaggtttttaagauga

SEQ ID NO: 76 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
gaagggtatgcatccagguu

SEQ ID NO: 77 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
aauuucttgaagagaagggu

SEQ ID NO: 78 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
gugaacacaaatttcuugaa

SEQ ID NO: 79 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
cauuuttcctttgtgaacac

SEQ ID NO: 80 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
caucccttcatgcatuuuuc

SEQ ID NO: 81 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
auggggtatccatcccuuca

SEQ ID NO: 82 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
augucatggaaaatggggua

SEQ ID NO: 83 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
guaauaatcatgtcauggaa

SEQ ID NO: 84 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
aggcatgcaatgtgtaauaa

SEQ ID NO: 85 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
guuuugatacaggcaugcaa

SEQ ID NO: 86 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
guacgtgagatgtttugaua

SEQ ID NO: 87 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
uauguttatgaggtacguga

SEQ ID NO: 88 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
cauaggtgtatatgtuuaug

SEQ ID NO: 89 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
uuugugggtacataggugua

SEQ ID NO: 90 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
auuaaaaaatttttgugggu

SEQ ID NO: 91 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
uuccuttttttaattaaaaa

SEQ ID NO: 92 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
aaacucaaatttcctuuuuu

SEQ ID NO: 93 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
uguuuctatttaaacucaaa

SEQ ID NO: 94 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
cuugcatttatcatguuucu

SEQ ID NO: 95 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
auguuttctttcttgcauuu

SEQ ID NO: 96 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
aaaaucaaaatgtttucuuu

SEQ ID NO: 97 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
gacaatgagttaaaaucaaa

SEQ ID NO: 98 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
gaacatcagagtgacaauga

SEQ ID NO: 99 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
caguucacatgaacaucaga

SEQ ID NO: 100 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
agcccgaagcaaccaguuca

SEQ ID NO: 101 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
cagaucaaagagcccgaagc

SEQ ID NO: 102 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
uccauaggtgacagaucaaa

SEQ ID NO: 103 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
accactcagattccauaggu

SEQ ID NO: 104 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
aaaaaataaaaccacucaga

SEQ ID NO: 105 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
cugagaaatctaaaaaauaa

SEQ ID NO: 106 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
uagauctttgggactgagaa

SEQ ID NO: 107 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
uuauutatcttagatcuuug

SEQ ID NO: 108 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
guucucttgtttattuaucu

SEQ ID NO: 109 - RNA, 20 nt, Artificial Sequence
  source: /note=“Description of Artificial Sequence: Synthetic oligonucleotide”
cuauccauggaauauuagaa

SEQ ID NO: 110 - RNA, 20 nt, Artificial Sequence
  source: /note=“Description of Artificial Sequence: Synthetic oligonucleotide”
uauuagaacuugacuugcuc

SEQ ID NO: 111 - RNA, 20 nt, Artificial Sequence
  source: /note=“Description of Artificial Sequence: Synthetic oligonucleotide”
uugacuugcuccauccucuu

SEQ ID NO: 112 - RNA, 20 nt, Artificial Sequence
  source: /note=“Description of Artificial Sequence: Synthetic oligonucleotide”
cauccucuuaaacuuuuugu

SEQ ID NO: 113 - RNA, 20 nt, Artificial Sequence
  source: /note=“Description of Artificial Sequence: Synthetic oligonucleotide”
uuuuugugucucacacuaaa

SEQ ID NO: 114 - RNA, 20 nt, Artificial Sequence
  source: /note=“Description of Artificial Sequence: Synthetic oligonucleotide”
cucacacuaaagaaaugaga

SEQ ID NO: 115 - RNA, 20 nt, Artificial Sequence
  source: /note=“Description of Artificial Sequence: Synthetic oligonucleotide”
aaaugagagaugcagaauuc

SEQ ID NO: 116 - RNA, 20 nt, Artificial Sequence
  source: /note=“Description of Artificial Sequence: Synthetic oligonucleotide”
cagaauucuaaggcuaaaua

SEQ ID NO: 117 - RNA, 20 nt, Artificial Sequence
  source: /note=“Description of Artificial Sequence: Synthetic oligonucleotide”
aggcuaaauagcuaggaagu

SEQ ID NO: 118 - RNA, 20 nt, Artificial Sequence
  source: /note=“Description of Artificial Sequence: Synthetic oligonucleotide”
uaggaaguauucauucaaac

SEQ ID NO: 119 - RNA, 20 nt, Artificial Sequence
  source: /note=“Description of Artificial Sequence: Synthetic oligonucleotide”
uucauucaaacuugaauauu

SEQ ID NO: 120 - RNA, 20 nt, Artificial Sequence
  source: /note=“Description of Artificial Sequence: Synthetic oligonucleotide”
uugaauauucuucaaagaga

SEQ ID NO: 121 - RNA, 20 nt, Artificial Sequence
  source: /note=“Description of Artificial Sequence: Synthetic oligonucleotide”
aaagagagugugggggcaac

SEQ ID NO: 122 - RNA, 20 nt, Artificial Sequence
  source: /note=“Description of Artificial Sequence: Synthetic oligonucleotide”
gugggggcaacucuaaucag

SEQ ID NO: 123 - RNA, 20 nt, Artificial Sequence
  source: /note=“Description of Artificial Sequence: Synthetic oligonucleotide”
cuaaucagaggaagaaacua

SEQ ID NO: 124 - RNA, 20 nt, Artificial Sequence
  source: /note=“Description of Artificial Sequence: Synthetic oligonucleotide”
aagaaacuaaaggaaguaaa

SEQ ID NO: 125 - RNA, 20 nt, Artificial Sequence
  source: /note=“Description of Artificial Sequence: Synthetic oligonucleotide”
aggaaguaaaaccagauguu

SEQ ID NO: 126 - RNA, 20 nt, Artificial Sequence
  source: /note=“Description of Artificial Sequence: Synthetic oligonucleotide”
agauguuuuccaccaaagcc

SEQ ID NO: 127 - RNA, 20 nt, Artificial Sequence
  source: /note=“Description of Artificial Sequence: Synthetic oligonucleotide”
accaaagcccuccuuuuggc

SEQ ID NO: 128 - RNA, 20 nt, Artificial Sequence
  source: /note=“Description of Artificial Sequence: Synthetic oligonucleotide”
uccuuuuggcuggucugauu

SEQ ID NO: 129 - RNA, 20 nt, Artificial Sequence
  source: /note=“Description of Artificial Sequence: Synthetic oligonucleotide”
uggucugauuucuaccgcac

SEQ ID NO: 130 - RNA, 20 nt, Artificial Sequence
  source: /note=“Description of Artificial Sequence: Synthetic oligonucleotide”
uaccgcacuggcagggccau

SEQ ID NO: 131 - RNA, 20 nt, Artificial Sequence
  source: /note=“Description of Artificial Sequence: Synthetic oligonucleotide”
cagggccaugggcuaauaua

SEQ ID NO: 132 - RNA, 20 nt, Artificial Sequence
  source: /note=“Description of Artificial Sequence: Synthetic oligonucleotide”
cuaauauaugugcuggcaag

SEQ ID NO: 133 - RNA, 20 nt, Artificial Sequence
  source: /note=“Description of Artificial Sequence: Synthetic oligonucleotide”
ugcuggcaagucuuccaaug

SEQ ID NO: 134 - RNA, 20 nt, Artificial Sequence
  source: /note=“Description of Artificial Sequence: Synthetic oligonucleotide”
uuccaaugagauccggacgu

SEQ ID NO: 135 - RNA, 20 nt, Artificial Sequence
  source: /note=“Description of Artificial Sequence: Synthetic oligonucleotide”
uccggacgugugaccgccau

SEQ ID NO: 136 - RNA, 20 nt, Artificial Sequence
  source: /note=“Description of Artificial Sequence: Synthetic oligonucleotide”
ugaccgccauggcuguggac

SEQ ID NO: 137 - RNA, 20 nt, Artificial Sequence
  source: /note=“Description of Artificial Sequence: Synthetic oligonucleotide”
cuguggacaguacucugcuc

SEQ ID NO: 138 - RNA, 20 nt, Artificial Sequence
  source: /note=“Description of Artificial Sequence: Synthetic oligonucleotide”
uacucugcucaaagaaguca

SEQ ID NO: 139 - RNA, 20 nt, Artificial Sequence
  source: /note=“Description of Artificial Sequence: Synthetic oligonucleotide”
aaagaagucagaggccucac

SEQ ID NO: 140 - RNA, 20 nt, Artificial Sequence
  source: /note=“Description of Artificial Sequence: Synthetic oligonucleotide”
aggccucaccagggugugga

SEQ ID NO: 141 - RNA, 20 nt, Artificial Sequence
  source: /note=“Description of Artificial Sequence: Synthetic oligonucleotide”
gguguggacaucuugugcuc

SEQ ID NO: 142 - RNA, 20 nt, Artificial Sequence
  source: /note=“Description of Artificial Sequence: Synthetic oligonucleotide”
ucuugugcucugcuggaucu

SEQ ID NO: 143 - RNA, 20 nt, Artificial Sequence
  source: /note=“Description of Artificial Sequence: Synthetic oligonucleotide”
cuggaucuacuguguacgca

SEQ ID NO: 144 - RNA, 20 nt, Artificial Sequence
  source: /note=“Description of Artificial Sequence: Synthetic oligonucleotide”
uguguacgcaccauucacug

SEQ ID NO: 145 - RNA, 20 nt, Artificial Sequence
  source: /note=“Description of Artificial Sequence: Synthetic oligonucleotide”
cauucacuggaaugauugug

SEQ ID NO: 146 - RNA, 20 nt, Artificial Sequence
  source: /note=“Description of Artificial Sequence: Synthetic oligonucleotide”
augauugugggccaggagaa

SEQ ID NO: 147 - RNA, 20 nt, Artificial Sequence
  source: /note=“Description of Artificial Sequence: Synthetic oligonucleotide”
aggagaaaccuuaucaaaac

SEQ ID NO: 148 - RNA, 20 nt, Artificial Sequence
  source: /note=“Description of Artificial Sequence: Synthetic oligonucleotide”
uuaucaaaacaagaaugcua

SEQ ID NO: 149 - RNA, 20 nt, Artificial Sequence
  source: /note=“Description of Artificial Sequence: Synthetic oligonucleotide”
aagaaugcuaucaauaaugg

SEQ ID NO: 150 - RNA, 20 nt, Artificial Sequence
  source: /note=“Description of Artificial Sequence: Synthetic oligonucleotide”
aauaaugguguucgaauauc

SEQ ID NO: 151 - RNA, 20 nt, Artificial Sequence
  source: /note=“Description of Artificial Sequence: Synthetic oligonucleotide”
cgaauaucuggaagagguuu

SEQ ID NO: 152 - RNA, 20 nt, Artificial Sequence
  source: /note=“Description of Artificial Sequence: Synthetic oligonucleotide”
aagagguuuuugugucaaaa

SEQ ID NO: 153 - RNA, 20 nt, Artificial Sequence
  source: /note=“Description of Artificial Sequence: Synthetic oligonucleotide”
uugugucaaaauguucuaca

SEQ ID NO: 154 - RNA, 20 nt, Artificial Sequence
  source: /note=“Description of Artificial Sequence: Synthetic oligonucleotide”
uucuacauuaagccaauuaa

SEQ ID NO: 155 - RNA, 20 nt, Artificial Sequence
  source: /note=“Description of Artificial Sequence: Synthetic oligonucleotide”
aagccaauuaaguauaaagg

SEQ ID NO: 156 - RNA, 20 nt, Artificial Sequence
  source: /note=“Description of Artificial Sequence: Synthetic oligonucleotide”
uauaaagguccuauuaagaa

SEQ ID NO: 157 - RNA, 20 nt, Artificial Sequence
  source: /note=“Description of Artificial Sequence: Synthetic oligonucleotide”
auuaagaagggagaaaaacu

SEQ ID NO: 158 - RNA, 20 nt, Artificial Sequence
  source: /note=“Description of Artificial Sequence: Synthetic oligonucleotide”
agaaaaacuuggaacucuau

SEQ ID NO: 159 - RNA, 20 nt, Artificial Sequence
  source: /note=“Description of Artificial Sequence: Synthetic oligonucleotide”
uggaacucuauugcccuugc

SEQ ID NO: 160 - RNA, 20 nt, Artificial Sequence
  source: /note=“Description of Artificial Sequence: Synthetic oligonucleotide”
ugcccuugcagaaaguuuau

SEQ ID NO: 161 - RNA, 20 nt, Artificial Sequence
  source: /note=“Description of Artificial Sequence: Synthetic oligonucleotide”
aguuuauccuggcauacaau

SEQ ID NO: 162 - RNA, 20 nt, Artificial Sequence
  source: /note=“Description of Artificial Sequence: Synthetic oligonucleotide”
uggcauacaaucgcaugugc

SEQ ID NO: 163 - RNA, 20 nt, Artificial Sequence
  source: /note=“Description of Artificial Sequence: Synthetic oligonucleotide”
gcaugugcacauugaaaacu

SEQ ID NO: 164 - RNA, 20 nt, Artificial Sequence
  source: /note=“Description of Artificial Sequence: Synthetic oligonucleotide”
uugaaaacugugacucgagu

SEQ ID NO: 165 - RNA, 20 nt, Artificial Sequence
  source: /note=“Description of Artificial Sequence: Synthetic oligonucleotide”
ugacucgagugacccuacug

SEQ ID NO: 166 - RNA, 20 nt, Artificial Sequence
  source: /note=“Description of Artificial Sequence: Synthetic oligonucleotide”
cccuacugcauaccuguaaa

SEQ ID NO: 167 - RNA, 20 nt, Artificial Sequence
  source: /note=“Description of Artificial Sequence: Synthetic oligonucleotide”
uaccuguaaaucgaaggcca

SEQ ID NO: 168 - RNA, 20 nt, Artificial Sequence
  source: /note=“Description of Artificial Sequence: Synthetic oligonucleotide”
aaggccaauggucagaucuu

SEQ ID NO: 169 - RNA, 20 nt, Artificial Sequence
  source: /note=“Description of Artificial Sequence: Synthetic oligonucleotide”
ucagaucuucaaaauaaaaa

SEQ ID NO: 170 - RNA, 20 nt, Artificial Sequence
  source: /note=“Description of Artificial Sequence: Synthetic oligonucleotide”
aaaauaaaaagucaucuuaa

SEQ ID NO: 171 - RNA, 20 nt, Artificial Sequence
  source: /note=“Description of Artificial Sequence: Synthetic oligonucleotide”
ucaucuuaaaaaccuggaug

SEQ ID NO: 172 - RNA, 20 nt, Artificial Sequence
  source: /note=“Description of Artificial Sequence: Synthetic oligonucleotide”
aaccuggaugcauacccuuc

SEQ ID NO: 173 - RNA, 20 nt, Artificial Sequence
  source: /note=“Description of Artificial Sequence: Synthetic oligonucleotide”
acccuucucuucaagaaauu

SEQ ID NO: 174 - RNA, 20 nt, Artificial Sequence
  source: /note=“Description of Artificial Sequence: Synthetic oligonucleotide”
uucaagaaauuuguguucac

SEQ ID NO: 175 - RNA, 20 nt, Artificial Sequence
  source: /note=“Description of Artificial Sequence: Synthetic oligonucleotide”
guguucacaaaggaaaaaug

SEQ ID NO: 176 - RNA, 20 nt, Artificial Sequence
  source: /note=“Description of Artificial Sequence: Synthetic oligonucleotide”
gaaaaaugcaugaagggaug

SEQ ID NO: 177 - RNA, 20 nt, Artificial Sequence
  source: /note=“Description of Artificial Sequence: Synthetic oligonucleotide”
ugaagggauggauaccccau

SEQ ID NO: 178 - RNA, 20 nt, Artificial Sequence
  source: /note=“Description of Artificial Sequence: Synthetic oligonucleotide”
uaccccauuuuccaugacau

SEQ ID NO: 179 - RNA, 20 nt, Artificial Sequence
  source: /note=“Description of Artificial Sequence: Synthetic oligonucleotide”
uuccaugacaugauuauuac

SEQ ID NO: 180 - RNA, 20 nt, Artificial Sequence
  source: /note=“Description of Artificial Sequence: Synthetic oligonucleotide”
uuauuacacauugcaugccu

SEQ ID NO: 181 - RNA, 20 nt, Artificial Sequence
  source: /note=“Description of Artificial Sequence: Synthetic oligonucleotide”
uugcaugccuguaucaaaac

SEQ ID NO: 182 - RNA, 20 nt, Artificial Sequence
  source: /note=“Description of Artificial Sequence: Synthetic oligonucleotide”
uaucaaaacaucucacguac

SEQ ID NO: 183 - RNA, 20 nt, Artificial Sequence
  source: /note=“Description of Artificial Sequence: Synthetic oligonucleotide”
ucacguaccucauaaacaua

SEQ ID NO: 184 - RNA, 20 nt, Artificial Sequence
  source: /note=“Description of Artificial Sequence: Synthetic oligonucleotide”
cauaaacauauacaccuaug

SEQ ID NO: 185 - RNA, 20 nt, Artificial Sequence
  source: /note=“Description of Artificial Sequence: Synthetic oligonucleotide”
uacaccuauguacccacaaa

SEQ ID NO: 186 - RNA, 20 nt, Artificial Sequence
  source: /note=“Description of Artificial Sequence: Synthetic oligonucleotide”
acccacaaaaauuuuuuaau

SEQ ID NO: 187 - RNA, 20 nt, Artificial Sequence
  source: /note=“Description of Artificial Sequence: Synthetic oligonucleotide”
uuuuuaauuaaaaaaaggaa

SEQ ID NO: 188 - RNA, 20 nt, Artificial Sequence
  source: /note=“Description of Artificial Sequence: Synthetic oligonucleotide”
aaaaaaggaaauuugaguuu

SEQ ID NO: 189 - RNA, 20 nt, Artificial Sequence
  source: /note=“Description of Artificial Sequence: Synthetic oligonucleotide”
uuugaguuuaaauagaaaca

SEQ ID NO: 190 - RNA, 20 nt, Artificial Sequence
  source: /note=“Description of Artificial Sequence: Synthetic oligonucleotide”
agaaacaugauaaaugcaag

SEQ ID NO: 191 - RNA, 20 nt, Artificial Sequence
  source: /note=“Description of Artificial Sequence: Synthetic oligonucleotide”
aaaugcaagaaagaaaacau

SEQ ID NO: 192 - RNA, 20 nt, Artificial Sequence
  source: /note=“Description of Artificial Sequence: Synthetic oligonucleotide”
aaagaaaacauuuugauuuu

SEQ ID NO: 193 - RNA, 20 nt, Artificial Sequence
  source: /note=“Description of Artificial Sequence: Synthetic oligonucleotide”
uuugauuuuaacucauuguc

SEQ ID NO: 194 - RNA, 20 nt, Artificial Sequence
  source: /note=“Description of Artificial Sequence: Synthetic oligonucleotide”
ucauugucacucugauguuc

SEQ ID NO: 195 - RNA, 20 nt, Artificial Sequence
  source: /note=“Description of Artificial Sequence: Synthetic oligonucleotide”
ucugauguucaugugaacug

SEQ ID NO: 196 - RNA, 20 nt, Artificial Sequence
  source: /note=“Description of Artificial Sequence: Synthetic oligonucleotide”
ugaacugguugcuucgggcu

SEQ ID NO: 197 - RNA, 20 nt, Artificial Sequence
  source: /note=“Description of Artificial Sequence: Synthetic oligonucleotide”
gcuucgggcucuuugaucug

SEQ ID NO: 198 - RNA, 20 nt, Artificial Sequence
  source: /note=“Description of Artificial Sequence: Synthetic oligonucleotide”
uuugaucugucaccuaugga

SEQ ID NO: 199 - RNA, 20 nt, Artificial Sequence
  source: /note=“Description of Artificial Sequence: Synthetic oligonucleotide”
accuauggaaucugaguggu

SEQ ID NO: 200 - RNA, 20 nt, Artificial Sequence
  source: /note=“Description of Artificial Sequence: Synthetic oligonucleotide”
ucugagugguuuuauuuuuu

SEQ ID NO: 201 - RNA, 20 nt, Artificial Sequence
  source: /note=“Description of Artificial Sequence: Synthetic oligonucleotide”
uuauuuuuuagauuucucag

SEQ ID NO: 202 - RNA, 20 nt, Artificial Sequence
  source: /note=“Description of Artificial Sequence: Synthetic oligonucleotide”
uucucagucccaaagaucua

SEQ ID NO: 203 - RNA, 20 nt, Artificial Sequence
  source: /note=“Description of Artificial Sequence: Synthetic oligonucleotide”
caaagaucuaagauaaauaa

SEQ ID NO: 204 - RNA, 20 nt, Artificial Sequence
  source: /note=“Description of Artificial Sequence: Synthetic oligonucleotide”
agauaaauaaacaagagaac

SEQ ID NO: 205 - RNA, 20 nt, Artificial Sequence
  source: /note=“Description of Artificial Sequence: Synthetic oligonucleotide”
uucuaauauuccauggauag

SEQ ID NO: 206 - RNA, 20 nt, Artificial Sequence
  source: /note=“Description of Artificial Sequence: Synthetic oligonucleotide”
gagcaagucaaguucuaaua

SEQ ID NO: 207 - RNA, 20 nt, Artificial Sequence
  source: /note=“Description of Artificial Sequence: Synthetic oligonucleotide”
aagaggauggagcaagucaa

SEQ ID NO: 208 - RNA, 20 nt, Artificial Sequence
  source: /note=“Description of Artificial Sequence: Synthetic oligonucleotide”
acaaaaaguuuaagaggaug

SEQ ID NO: 209 - RNA, 20 nt, Artificial Sequence
  source: /note=“Description of Artificial Sequence: Synthetic oligonucleotide”
uuuagugugagacacaaaaa

SEQ ID NO: 210 - RNA, 20 nt, Artificial Sequence
  source: /note=“Description of Artificial Sequence: Synthetic oligonucleotide”
ucucauuucuuuagugugag

SEQ ID NO: 211 - RNA, 20 nt, Artificial Sequence
  source: /note=“Description of Artificial Sequence: Synthetic oligonucleotide”
gaauucugcaucucucauuu

SEQ ID NO: 212 - RNA, 20 nt, Artificial Sequence
  source: /note=“Description of Artificial Sequence: Synthetic oligonucleotide”
uauuuagccuuagaauucug

SEQ ID NO: 213 - RNA, 20 nt, Artificial Sequence
  source: /note=“Description of Artificial Sequence: Synthetic oligonucleotide”
acuuccuagcuauuuagccu

SEQ ID NO: 214 - RNA, 20 nt, Artificial Sequence
  source: /note=“Description of Artificial Sequence: Synthetic oligonucleotide”
guuugaaugaauacuuccua

SEQ ID NO: 215 - RNA, 20 nt, Artificial Sequence
  source: /note=“Description of Artificial Sequence: Synthetic oligonucleotide”
aauauucaaguuugaaugaa

SEQ ID NO: 216 - RNA, 20 nt, Artificial Sequence
  source: /note=“Description of Artificial Sequence: Synthetic oligonucleotide”
ucucuuugaagaauauucaa

SEQ ID NO: 217 - RNA, 20 nt, Artificial Sequence
  source: /note=“Description of Artificial Sequence: Synthetic oligonucleotide”
guugcccccacacucucuuu

SEQ ID NO: 218 - RNA, 20 nt, Artificial Sequence
  source: /note=“Description of Artificial Sequence: Synthetic oligonucleotide”
cugauuagaguugcccccac

SEQ ID NO: 219 - RNA, 20 nt, Artificial Sequence
  source: /note=“Description of Artificial Sequence: Synthetic oligonucleotide”
uaguuucuuccucugauuag

SEQ ID NO: 220 - RNA, 20 nt, Artificial Sequence
  source: /note=“Description of Artificial Sequence: Synthetic oligonucleotide”
uuuacuuccuuuaguuucuu

SEQ ID NO: 221 - RNA, 20 nt, Artificial Sequence
  source: /note=“Description of Artificial Sequence: Synthetic oligonucleotide”
aacaucugguuuuacuuccu

SEQ ID NO: 222 - RNA, 20 nt, Artificial Sequence
  source: /note=“Description of Artificial Sequence: Synthetic oligonucleotide”
ggcuuugguggaaaacaucu

SEQ ID NO: 223 - RNA, 20 nt, Artificial Sequence
  source: /note=“Description of Artificial Sequence: Synthetic oligonucleotide”
gccaaaaggagggcuuuggu

SEQ ID NO: 224 - RNA, 20 nt, Artificial Sequence
  source: /note=“Description of Artificial Sequence: Synthetic oligonucleotide”
aaucagaccagccaaaagga

SEQ ID NO: 225 - RNA, 20 nt, Artificial Sequence
  source: /note=“Description of Artificial Sequence: Synthetic oligonucleotide”
gugcgguagaaaucagacca

SEQ ID NO: 226 - RNA, 20 nt, Artificial Sequence
  source: /note=“Description of Artificial Sequence: Synthetic oligonucleotide”
auggcccugccagugcggua

SEQ ID NO: 227 - RNA, 20 nt, Artificial Sequence
  source: /note=“Description of Artificial Sequence: Synthetic oligonucleotide”
uauauuagcccauggcccug

SEQ ID NO: 228 - RNA, 20 nt, Artificial Sequence
  source: /note=“Description of Artificial Sequence: Synthetic oligonucleotide”
cuugccagcacauauauuag

SEQ ID NO: 229 - RNA, 20 nt, Artificial Sequence
  source: /note=“Description of Artificial Sequence: Synthetic oligonucleotide”
cauuggaagacuugccagca

SEQ ID NO: 230 - RNA, 20 nt, Artificial Sequence
  source: /note=“Description of Artificial Sequence: Synthetic oligonucleotide”
acguccggaucucauuggaa

SEQ ID NO: 231 - RNA, 20 nt, Artificial Sequence
  source: /note=“Description of Artificial Sequence: Synthetic oligonucleotide”
auggcggucacacguccgga

SEQ ID NO: 232 - RNA, 20 nt, Artificial Sequence
  source: /note=“Description of Artificial Sequence: Synthetic oligonucleotide”
guccacagccauggcgguca

SEQ ID NO: 233 - RNA, 20 nt, Artificial Sequence
  source: /note=“Description of Artificial Sequence: Synthetic oligonucleotide”
gagcagaguacuguccacag

SEQ ID NO: 234 - RNA, 20 nt, Artificial Sequence
  source: /note=“Description of Artificial Sequence: Synthetic oligonucleotide”
ugacuucuuugagcagagua

SEQ ID NO: 235 - RNA, 20 nt, Artificial Sequence
  source: /note=“Description of Artificial Sequence: Synthetic oligonucleotide”
gugaggccucugacuucuuu

SEQ ID NO: 236 - RNA, 20 nt, Artificial Sequence
  source: /note=“Description of Artificial Sequence: Synthetic oligonucleotide”
uccacacccuggugaggccu

SEQ ID NO: 237 - RNA, 20 nt, Artificial Sequence
  source: /note=“Description of Artificial Sequence: Synthetic oligonucleotide”
gagcacaagauguccacacc

SEQ ID NO: 238 - RNA, 20 nt, Artificial Sequence
  source: /note=“Description of Artificial Sequence: Synthetic oligonucleotide”
agauccagcagagcacaaga

SEQ ID NO: 239 - RNA, 20 nt, Artificial Sequence
  source: /note=“Description of Artificial Sequence: Synthetic oligonucleotide”
ugcguacacaguagauccag

SEQ ID NO: 240 - RNA, 20 nt, Artificial Sequence
  source: /note=“Description of Artificial Sequence: Synthetic oligonucleotide”
cagugaauggugcguacaca

SEQ ID NO: 241 - RNA, 20 nt, Artificial Sequence
  source: /note=“Description of Artificial Sequence: Synthetic oligonucleotide”
cacaaucauuccagugaaug

SEQ ID NO: 242 - RNA, 20 nt, Artificial Sequence
  source: /note=“Description of Artificial Sequence: Synthetic oligonucleotide”
uucuccuggcccacaaucau

SEQ ID NO: 243 - RNA, 20 nt, Artificial Sequence
  source: /note=“Description of Artificial Sequence: Synthetic oligonucleotide”
guuuugauaagguuucuccu

SEQ ID NO: 244 - RNA, 20 nt, Artificial Sequence
  source: /note=“Description of Artificial Sequence: Synthetic oligonucleotide”
uagcauucuuguuuugauaa

SEQ ID NO: 245 - RNA, 20 nt, Artificial Sequence
  source: /note=“Description of Artificial Sequence: Synthetic oligonucleotide”
ccauuauugauagcauucuu

SEQ ID NO: 246 - RNA, 20 nt, Artificial Sequence
  source: /note=“Description of Artificial Sequence: Synthetic oligonucleotide”
gauauucgaacaccauuauu

SEQ ID NO: 247 - RNA, 20 nt, Artificial Sequence
  source: /note=“Description of Artificial Sequence: Synthetic oligonucleotide”
aaaccucuuccagauauucg

SEQ ID NO: 248 - RNA, 20 nt, Artificial Sequence
  source: /note=“Description of Artificial Sequence: Synthetic oligonucleotide”
uuuugacacaaaaaccucuu

SEQ ID NO: 249 - RNA, 20 nt, Artificial Sequence
  source: /note=“Description of Artificial Sequence: Synthetic oligonucleotide”
uguagaacauuuugacacaa

SEQ ID NO: 250 - RNA, 20 nt, Artificial Sequence
  source: /note=“Description of Artificial Sequence: Synthetic oligonucleotide”
uuaauuggcuuaauguagaa

SEQ ID NO: 251 - RNA, 20 nt, Artificial Sequence
  source: /note=“Description of Artificial Sequence: Synthetic oligonucleotide”
ccuuuauacuuaauuggcuu

SEQ ID NO: 252 - RNA, 20 nt, Artificial Sequence
  source: /note=“Description of Artificial Sequence: Synthetic oligonucleotide”
uucuuaauaggaccuuuaua

SEQ ID NO: 253 - RNA, 20 nt, Artificial Sequence
  source: /note=“Description of Artificial Sequence: Synthetic oligonucleotide”
aguuuuucucccuucuuaau

SEQ ID NO: 254 - RNA, 20 nt, Artificial Sequence
  source: /note=“Description of Artificial Sequence: Synthetic oligonucleotide”
auagaguuccaaguuuuucu

SEQ ID NO: 255 - RNA, 20 nt, Artificial Sequence
  source: /note=“Description of Artificial Sequence: Synthetic oligonucleotide”
gcaagggcaauagaguucca

SEQ ID NO: 256 - RNA, 20 nt, Artificial Sequence
  source: /note=“Description of Artificial Sequence: Synthetic oligonucleotide”
auaaacuuucugcaagggca

SEQ ID NO: 257 - RNA, 20 nt, Artificial Sequence
  source: /note=“Description of Artificial Sequence: Synthetic oligonucleotide”
auuguaugccaggauaaacu

SEQ ID NO: 258 - RNA, 20 nt, Artificial Sequence
  source: /note=“Description of Artificial Sequence: Synthetic oligonucleotide”
gcacaugcgauuguaugcca

SEQ ID NO: 259 - RNA, 20 nt, Artificial Sequence
  source: /note=“Description of Artificial Sequence: Synthetic oligonucleotide”
aguuuucaaugugcacaugc

SEQ ID NO: 260 - RNA, 20 nt, Artificial Sequence
  source: /note=“Description of Artificial Sequence: Synthetic oligonucleotide”
acucgagucacaguuuucaa

SEQ ID NO: 261 - RNA, 20 nt, Artificial Sequence
  source: /note=“Description of Artificial Sequence: Synthetic oligonucleotide”
caguagggucacucgaguca

SEQ ID NO: 262 - RNA, 20 nt, Artificial Sequence
  source: /note=“Description of Artificial Sequence: Synthetic oligonucleotide”
uuuacagguaugcaguaggg

SEQ ID NO: 263 - RNA, 20 nt, Artificial Sequence
  source: /note=“Description of Artificial Sequence: Synthetic oligonucleotide”
uggccuucgauuuacaggua

SEQ ID NO: 264 - RNA, 20 nt, Artificial Sequence
  source: /note=“Description of Artificial Sequence: Synthetic oligonucleotide”
aagaucugaccauuggccuu

SEQ ID NO: 265 - RNA, 20 nt, Artificial Sequence
  source: /note=“Description of Artificial Sequence: Synthetic oligonucleotide”
uuuuuauuuugaagaucuga

SEQ ID NO: 266 - RNA, 20 nt, Artificial Sequence
  source: /note=“Description of Artificial Sequence: Synthetic oligonucleotide”
uuaagaugacuuuuuauuuu

SEQ ID NO: 267 - RNA, 20 nt, Artificial Sequence
  source: /note=“Description of Artificial Sequence: Synthetic oligonucleotide”
cauccagguuuuuaagauga

SEQ ID NO: 268 - RNA, 20 nt, Artificial Sequence
  source: /note=“Description of Artificial Sequence: Synthetic oligonucleotide”
gaaggguaugcauccagguu

SEQ ID NO: 269 - RNA, 20 nt, Artificial Sequence
  source: /note=“Description of Artificial Sequence: Synthetic oligonucleotide”
aauuucuugaagagaagggu

SEQ ID NO: 270 - RNA, 20 nt, Artificial Sequence
  source: /note=“Description of Artificial Sequence: Synthetic oligonucleotide”
gugaacacaaauuucuugaa

SEQ ID NO: 271 - RNA, 20 nt, Artificial Sequence
  source: /note=“Description of Artificial Sequence: Synthetic oligonucleotide”
cauuuuuccuuugugaacac

SEQ ID NO: 272 - RNA, 20 nt, Artificial Sequence
  source: /note=“Description of Artificial Sequence: Synthetic oligonucleotide”
caucccuucaugcauuuuuc

SEQ ID NO: 273 - RNA, 20 nt, Artificial Sequence
  source: /note=“Description of Artificial Sequence: Synthetic oligonucleotide”
augggguauccaucccuuca

SEQ ID NO: 274 - RNA, 20 nt, Artificial Sequence
  source: /note=“Description of Artificial Sequence: Synthetic oligonucleotide”
augucauggaaaauggggua

SEQ ID NO: 275 - RNA, 20 nt, Artificial Sequence
  source: /note=“Description of Artificial Sequence: Synthetic oligonucleotide”
guaauaaucaugucauggaa

SEQ ID NO: 276 - RNA, 20 nt, Artificial Sequence
  source: /note=“Description of Artificial Sequence: Synthetic oligonucleotide”
aggcaugcaauguguaauaa

SEQ ID NO: 277 - RNA, 20 nt, Artificial Sequence
  source: /note=“Description of Artificial Sequence: Synthetic oligonucleotide”
guuuugauacaggcaugcaa

SEQ ID NO: 278 - RNA, 20 nt, Artificial Sequence
  source: /note=“Description of Artificial Sequence: Synthetic oligonucleotide”
guacgugagauguuuugaua

SEQ ID NO: 279 - RNA, 20 nt, Artificial Sequence
  source: /note=“Description of Artificial Sequence: Synthetic oligonucleotide”
uauguuuaugagguacguga

SEQ ID NO: 280 - RNA, 20 nt, Artificial Sequence
  source: /note=“Description of Artificial Sequence: Synthetic oligonucleotide”
cauagguguauauguuuaug

SEQ ID NO: 281 - RNA, 20 nt, Artificial Sequence
  source: /note=“Description of Artificial Sequence: Synthetic oligonucleotide”
uuuguggguacauaggugua

SEQ ID NO: 282 - RNA, 20 nt, Artificial Sequence
  source: /note=“Description of Artificial Sequence: Synthetic oligonucleotide”
auuaaaaaauuuuugugggu

SEQ ID NO: 283 - RNA, 20 nt, Artificial Sequence
  source: /note=“Description of Artificial Sequence: Synthetic oligonucleotide”
uuccuuuuuuuaauuaaaaa

SEQ ID NO: 284 - RNA, 20 nt, Artificial Sequence
  source: /note=“Description of Artificial Sequence: Synthetic oligonucleotide”
aaacucaaauuuccuuuuuu

SEQ ID NO: 285 - RNA, 20 nt, Artificial Sequence
  source: /note=“Description of Artificial Sequence: Synthetic oligonucleotide”
uguuucuauuuaaacucaaa

SEQ ID NO: 286 - RNA, 20 nt, Artificial Sequence
  source: /note=“Description of Artificial Sequence: Synthetic oligonucleotide”
cuugcauuuaucauguuucu

SEQ ID NO: 287 - RNA, 20 nt, Artificial Sequence
  source: /note=“Description of Artificial Sequence: Synthetic oligonucleotide”
auguuuucuuucuugcauuu

SEQ ID NO: 288 - RNA, 20 nt, Artificial Sequence
  source: /note=“Description of Artificial Sequence: Synthetic oligonucleotide”
aaaaucaaaauguuuucuuu

SEQ ID NO: 289 - RNA, 20 nt, Artificial Sequence
  source: /note=“Description of Artificial Sequence: Synthetic oligonucleotide”
gacaaugaguuaaaaucaaa

SEQ ID NO: 290 - RNA, 20 nt, Artificial Sequence
  source: /note=“Description of Artificial Sequence: Synthetic oligonucleotide”
gaacaucagagugacaauga

SEQ ID NO: 291 - RNA, 20 nt, Artificial Sequence
  source: /note=“Description of Artificial Sequence: Synthetic oligonucleotide”
caguucacaugaacaucaga

SEQ ID NO: 292 - RNA, 20 nt, Artificial Sequence
  source: /note=“Description of Artificial Sequence: Synthetic oligonucleotide”
agcccgaagcaaccaguuca

SEQ ID NO: 293 - RNA, 20 nt, Artificial Sequence
  source: /note=“Description of Artificial Sequence: Synthetic oligonucleotide”
cagaucaaagagcccgaagc

SEQ ID NO: 294 - RNA, 20 nt, Artificial Sequence
  source: /note=“Description of Artificial Sequence: Synthetic oligonucleotide”
uccauaggugacagaucaaa

SEQ ID NO: 295 - RNA, 20 nt, Artificial Sequence
  source: /note=“Description of Artificial Sequence: Synthetic oligonucleotide”
accacucagauuccauaggu

SEQ ID NO: 296 - RNA, 20 nt, Artificial Sequence
  source: /note=“Description of Artificial Sequence: Synthetic oligonucleotide”
aaaaaauaaaaccacucaga

SEQ ID NO: 297 - RNA, 20 nt, Artificial Sequence
  source: /note=“Description of Artificial Sequence: Synthetic oligonucleotide”
cugagaaaucuaaaaaauaa

SEQ ID NO: 298 - RNA, 20 nt, Artificial Sequence
  source: /note=“Description of Artificial Sequence: Synthetic oligonucleotide”
uagaucuuugggacugagaa

SEQ ID NO: 299 - RNA, 20 nt, Artificial Sequence
  source: /note=“Description of Artificial Sequence: Synthetic oligonucleotide”
uuauuuaucuuagaucuuug

SEQ ID NO: 300 - RNA, 20 nt, Artificial Sequence
  source: /note=“Description of Artificial Sequence: Synthetic oligonucleotide”
guucucuuguuuauuuaucu

SEQ ID NO: 301-496 - skipped (196 entries)

SEQ ID NO: 497 - RNA, 21 nt, Artificial Sequence
  source: /note=“Description of Artificial Sequence: Synthetic oligonucleotide”
uagcuaggaaguauucauuca

SEQ ID NO: 498 - RNA, 23 nt, Artificial Sequence
  source: /note=“Description of Artificial Sequence: Synthetic oligonucleotide”
ugaaugaauacuuccuagcuauu

SEQ ID NO: 499 - RNA, 21 nt, Artificial Sequence
  source: /note=“Description of Artificial Sequence: Synthetic oligonucleotide”
agcuaggaaguauucauucaa

SEQ ID NO: 500 - RNA, 23 nt, Artificial Sequence
  source: /note=“Description of Artificial Sequence: Synthetic oligonucleotide”
uugaaugaauacuuccuagcuau

SEQ ID NO: 501 - RNA, 21 nt, Artificial Sequence
  source: /note=“Description of Artificial Sequence: Synthetic oligonucleotide”
auucauucaaacuugaauauu

SEQ ID NO: 502 - RNA, 23 nt, Artificial Sequence
  source: /note=“Description of Artificial Sequence: Synthetic oligonucleotide”
aauauucaaguuugaaugaauac

SEQ ID NO: 503 - RNA, 21 nt, Artificial Sequence
  source: /note=“Description of Artificial Sequence: Synthetic oligonucleotide”
uucaaacuugaauauucuuca

SEQ ID NO: 504 - RNA, 23 nt, Artificial Sequence
  source: /note=“Description of Artificial Sequence: Synthetic oligonucleotide”
ugaagaauauucaaguuugaaug

SEQ ID NO: 505 - RNA, 21 nt, Artificial Sequence
  source: /note=“Description of Artificial Sequence: Synthetic oligonucleotide”
uugaauauucuucaaagagag

SEQ ID NO: 506 - RNA, 23 nt, Artificial Sequence
  source: /note=“Description of Artificial Sequence: Synthetic oligonucleotide”
cucucuuugaagaauauucaagu

SEQ ID NO: 507 - RNA, 21 nt, Artificial Sequence
  source: /note=“Description of Artificial Sequence: Synthetic oligonucleotide”
uugaauauucuucaaagagaa

SEQ ID NO: 508 - RNA, 23 nt, Artificial Sequence
  source: /note=“Description of Artificial Sequence: Synthetic oligonucleotide”
uucucuuugaagaauauucaagu

SEQ ID NO: 509 - RNA, 21 nt, Artificial Sequence
  source: /note=“Description of Artificial Sequence: Synthetic oligonucleotide”
gcuaauauaugugcuggcaag

SEQ ID NO: 510 - RNA, 23 nt, Artificial Sequence
  source: /note=“Description of Artificial Sequence: Synthetic oligonucleotide”
cuugccagcacauauauuagccc

SEQ ID NO: 511 - RNA, 21 nt, Artificial Sequence
  source: /note=“Description of Artificial Sequence: Synthetic oligonucleotide”
gcuaauauaugugcuggcaaa

SEQ ID NO: 512 - RNA, 23 nt, Artificial Sequence
  source: /note=“Description of Artificial Sequence: Synthetic oligonucleotide”
uuugccagcacauauauuagccc

SEQ ID NO: 513 - RNA, 21 nt, Artificial Sequence
  source: /note=“Description of Artificial Sequence: Synthetic oligonucleotide”
cauggcuguggacaguacucu

SEQ ID NO: 514 - RNA, 23 nt, Artificial Sequence
  source: /note=“Description of Artificial Sequence: Synthetic oligonucleotide”
agaguacuguccacagccauggc

SEQ ID NO: 515 - RNA, 21 nt, Artificial Sequence
  source: /note=“Description of Artificial Sequence: Synthetic oligonucleotide”
uggcuguggacaguacucugc

SEQ ID NO: 516 - RNA, 23 nt, Artificial Sequence
  source: /note=“Description of Artificial Sequence: Synthetic oligonucleotide”
gcagaguacuguccacagccaug

SEQ ID NO: 517 - RNA, 21 nt, Artificial Sequence
  source: /note=“Description of Artificial Sequence: Synthetic oligonucleotide”
uggcuguggacaguacucuga

SEQ ID NO: 518 - RNA, 23 nt, Artificial Sequence
  source: /note=“Description of Artificial Sequence: Synthetic oligonucleotide”
ucagaguacuguccacagccaug

SEQ ID NO: 519 - RNA, 21 nt, Artificial Sequence
  source: /note=“Description of Artificial Sequence: Synthetic oligonucleotide”
ggcuguggacaguacucugcu

SEQ ID NO: 520 - RNA, 23 nt, Artificial Sequence
  source: /note=“Description of Artificial Sequence: Synthetic oligonucleotide”
agcagaguacuguccacagccau

SEQ ID NO: 521 - RNA, 21 nt, Artificial Sequence
  source: /note=“Description of Artificial Sequence: Synthetic oligonucleotide”
gcuguggacaguacucugcuc

SEQ ID NO: 522 - RNA, 23 nt, Artificial Sequence
  source: /note=“Description of Artificial Sequence: Synthetic oligonucleotide”
gagcagaguacuguccacagcca

SEQ ID NO: 523 - RNA, 21 nt, Artificial Sequence
  source: /note=“Description of Artificial Sequence: Synthetic oligonucleotide”
gcuguggacaguacucugcua

SEQ ID NO: 524 - RNA, 23 nt, Artificial Sequence
  source: /note=“Description of Artificial Sequence: Synthetic oligonucleotide”
uagcagaguacuguccacagcca

SEQ ID NO: 525 - RNA, 21 nt, Artificial Sequence
  source: /note=“Description of Artificial Sequence: Synthetic oligonucleotide”
cuguggacaguacucugcuca

SEQ ID NO: 526 - RNA, 23 nt, Artificial Sequence
  source: /note=“Description of Artificial Sequence: Synthetic oligonucleotide”
ugagcagaguacuguccacagcc

SEQ ID NO: 527 - RNA, 21 nt, Artificial Sequence
  source: /note=“Description of Artificial Sequence: Synthetic oligonucleotide”
uguggacaguacucugcucaa

SEQ ID NO: 528 - RNA, 23 nt, Artificial Sequence
  source: /note=“Description of Artificial Sequence: Synthetic oligonucleotide”
uugagcagaguacuguccacagc

SEQ ID NO: 529 - RNA, 21 nt, Artificial Sequence
  source: /note=“Description of Artificial Sequence: Synthetic oligonucleotide”
guggacaguacucugcucaaa

SEQ ID NO: 530 - RNA, 23 nt, Artificial Sequence
  source: /note=“Description of Artificial Sequence: Synthetic oligonucleotide”
uuugagcagaguacuguccacag

SEQ ID NO: 531 - RNA, 21 nt, Artificial Sequence
  source: /note=“Description of Artificial Sequence: Synthetic oligonucleotide”
uggacaguacucugcucaaag

SEQ ID NO: 532 - RNA, 23 nt, Artificial Sequence
  source: /note=“Description of Artificial Sequence: Synthetic oligonucleotide”
cuuugagcagaguacuguccaca

SEQ ID NO: 533 - RNA, 21 nt, Artificial Sequence
  source: /note=“Description of Artificial Sequence: Synthetic oligonucleotide”
uggacaguacucugcucaaau

SEQ ID NO: 534 - RNA, 23 nt, Artificial Sequence
  source: /note=“Description of Artificial Sequence: Synthetic oligonucleotide”
auuugagcagaguacuguccaca

SEQ ID NO: 535 - RNA, 21 nt, Artificial Sequence
  source: /note=“Description of Artificial Sequence: Synthetic oligonucleotide”
ggacaguacucugcucaaaga

SEQ ID NO: 536 - RNA, 23 nt, Artificial Sequence
  source: /note=“Description of Artificial Sequence: Synthetic oligonucleotide”
ucuuugagcagaguacuguccac

SEQ ID NO: 537 - RNA, 21 nt, Artificial Sequence
  source: /note=“Description of Artificial Sequence: Synthetic oligonucleotide”
gacaguacucugcucaaagaa

SEQ ID NO: 538 - RNA, 23 nt, Artificial Sequence
  source: /note=“Description of Artificial Sequence: Synthetic oligonucleotide”
uucuuugagcagaguacugucca

SEQ ID NO: 539 - RNA, 21 nt, Artificial Sequence
  source: /note=“Description of Artificial Sequence: Synthetic oligonucleotide”
ugugggccaggagaaaccuua

SEQ ID NO: 540 - RNA, 23 nt, Artificial Sequence
  source: /note=“Description of Artificial Sequence: Synthetic oligonucleotide”
uaagguuucuccuggcccacaau

SEQ ID NO: 541 - RNA, 21 nt, Artificial Sequence
  source: /note=“Description of Artificial Sequence: Synthetic oligonucleotide”
ccaggagaaaccuuaucaaaa

SEQ ID NO: 542 - RNA, 23 nt, Artificial Sequence
  source: /note=“Description of Artificial Sequence: Synthetic oligonucleotide”
uuuugauaagguuucuccuggcc

SEQ ID NO: 543 - RNA, 21 nt, Artificial Sequence
  source: /note=“Description of Artificial Sequence: Synthetic oligonucleotide”
uguucuacauuaagccaauua

SEQ ID NO: 544 - RNA, 23 nt, Artificial Sequence
  source: /note=“Description of Artificial Sequence: Synthetic oligonucleotide”
uaauuggcuuaauguagaacauu

SEQ ID NO: 545 - RNA, 21 nt, Artificial Sequence
  source: /note=“Description of Artificial Sequence: Synthetic oligonucleotide”
guucuacauuaagccaauuaa

SEQ ID NO: 546 - RNA, 23 nt, Artificial Sequence
  source: /note=“Description of Artificial Sequence: Synthetic oligonucleotide”
uuaauuggcuuaauguagaacau

SEQ ID NO: 547 - RNA, 21 nt, Artificial Sequence
  source: /note=“Description of Artificial Sequence: Synthetic oligonucleotide”
uucuacauuaagccaauuaag

SEQ ID NO: 548 - RNA, 23 nt, Artificial Sequence
  source: /note=“Description of Artificial Sequence: Synthetic oligonucleotide”
cuuaauuggcuuaauguagaaca

SEQ ID NO: 549 - RNA, 21 nt, Artificial Sequence
  source: /note=“Description of Artificial Sequence: Synthetic oligonucleotide”
uucuacauuaagccaauuaaa

SEQ ID NO: 550 - RNA, 23 nt, Artificial Sequence
  source: /note=“Description of Artificial Sequence: Synthetic oligonucleotide”
uuuaauuggcuuaauguagaaca

SEQ ID NO: 551 - RNA, 21 nt, Artificial Sequence
  source: /note=“Description of Artificial Sequence: Synthetic oligonucleotide”
ucuacauuaagccaauuaagu

SEQ ID NO: 552 - RNA, 23 nt, Artificial Sequence
  source: /note=“Description of Artificial Sequence: Synthetic oligonucleotide”
acuuaauuggcuuaauguagaac

SEQ ID NO: 553 - RNA, 21 nt, Artificial Sequence
  source: /note=“Description of Artificial Sequence: Synthetic oligonucleotide”
cuacauuaagccaauuaagua

SEQ ID NO: 554 - RNA, 23 nt, Artificial Sequence
  source: /note=“Description of Artificial Sequence: Synthetic oligonucleotide”
uacuuaauuggcuuaauguagaa

SEQ ID NO: 555 - RNA, 21 nt, Artificial Sequence
  source: /note=“Description of Artificial Sequence: Synthetic oligonucleotide”
uacauuaagccaauuaaguau

SEQ ID NO: 556 - RNA, 23 nt, Artificial Sequence
  source: /note=“Description of Artificial Sequence: Synthetic oligonucleotide”
auacuuaauuggcuuaauguaga

SEQ ID NO: 557 - RNA, 21 nt, Artificial Sequence
  source: /note=“Description of Artificial Sequence: Synthetic oligonucleotide”
acauuaagccaauuaaguaua

SEQ ID NO: 558 - RNA, 23 nt, Artificial Sequence
  source: /note=“Description of Artificial Sequence: Synthetic oligonucleotide”
uauacuuaauuggcuuaauguag

SEQ ID NO: 559 - RNA, 21 nt, Artificial Sequence
  source: /note=“Description of Artificial Sequence: Synthetic oligonucleotide”
cauuaagccaauuaaguauaa

SEQ ID NO: 560 - RNA, 23 nt, Artificial Sequence
  source: /note=“Description of Artificial Sequence: Synthetic oligonucleotide”
uuauacuuaauuggcuuaaugua

SEQ ID NO: 561 - RNA, 21 nt, Artificial Sequence
  source: /note=“Description of Artificial Sequence: Synthetic oligonucleotide”
auuaagccaauuaaguauaaa

SEQ ID NO: 562 - RNA, 23 nt, Artificial Sequence
  source: /note=“Description of Artificial Sequence: Synthetic oligonucleotide”
uuuauacuuaauuggcuuaaugu

SEQ ID NO: 563 - RNA, 21 nt, Artificial Sequence
  source: /note=“Description of Artificial Sequence: Synthetic oligonucleotide”
uuaagccaauuaaguauaaag

SEQ ID NO: 564 - RNA, 23 nt, Artificial Sequence
  source: /note=“Description of Artificial Sequence: Synthetic oligonucleotide”
cuuuauacuuaauuggcuuaaug

SEQ ID NO: 565 - RNA, 21 nt, Artificial Sequence
  source: /note=“Description of Artificial Sequence: Synthetic oligonucleotide”
uuaagccaauuaaguauaaau

SEQ ID NO: 566 - RNA, 23 nt, Artificial Sequence
  source: /note=“Description of Artificial Sequence: Synthetic oligonucleotide”
auuuauacuuaauuggcuuaaug

SEQ ID NO: 567 - RNA, 21 nt, Artificial Sequence
  source: /note=“Description of Artificial Sequence: Synthetic oligonucleotide”
uaagccaauuaaguauaaagg

SEQ ID NO: 568 - RNA, 23 nt, Artificial Sequence
  source: /note=“Description of Artificial Sequence: Synthetic oligonucleotide”
ccuuuauacuuaauuggcuuaau

SEQ ID NO: 569 - RNA, 21 nt, Artificial Sequence
  source: /note=“Description of Artificial Sequence: Synthetic oligonucleotide”
uaagccaauuaaguauaaaga

SEQ ID NO: 570 - RNA, 23 nt, Artificial Sequence
  source: /note=“Description of Artificial Sequence: Synthetic oligonucleotide”
ucuuuauacuuaauuggcuuaau

SEQ ID NO: 571 - RNA, 21 nt, Artificial Sequence
  source: /note=“Description of Artificial Sequence: Synthetic oligonucleotide”
aagccaauuaaguauaaaggu

SEQ ID NO: 572 - RNA, 23 nt, Artificial Sequence
  source: /note=“Description of Artificial Sequence: Synthetic oligonucleotide”
accuuuauacuuaauuggcuuaa

SEQ ID NO: 573 - RNA, 21 nt, Artificial Sequence
  source: /note=“Description of Artificial Sequence: Synthetic oligonucleotide”
agccaauuaaguauaaagguc

SEQ ID NO: 574 - RNA, 23 nt, Artificial Sequence
  source: /note=“Description of Artificial Sequence: Synthetic oligonucleotide”
gaccuuuauacuuaauuggcuua

SEQ ID NO: 575 - RNA, 21 nt, Artificial Sequence
  source: /note=“Description of Artificial Sequence: Synthetic oligonucleotide”
agccaauuaaguauaaaggua

SEQ ID NO: 576 - RNA, 23 nt, Artificial Sequence
  source: /note=“Description of Artificial Sequence: Synthetic oligonucleotide”
uaccuuuauacuuaauuggcuua

SEQ ID NO: 577 - RNA, 21 nt, Artificial Sequence
  source: /note=“Description of Artificial Sequence: Synthetic oligonucleotide”
gcccuugcagaaaguuuaucc

SEQ ID NO: 578 - RNA, 23 nt, Artificial Sequence
  source: /note=“Description of Artificial Sequence: Synthetic oligonucleotide”
ggauaaacuuucugcaagggcaa

SEQ ID NO: 579 - RNA, 21 nt, Artificial Sequence
  source: /note=“Description of Artificial Sequence: Synthetic oligonucleotide”
gcccuugcagaaaguuuauca

SEQ ID NO: 580 - RNA, 23 nt, Artificial Sequence
  source: /note=“Description of Artificial Sequence: Synthetic oligonucleotide”
ugauaaacuuucugcaagggcaa

SEQ ID NO: 581 - RNA, 21 nt, Artificial Sequence
  source: /note=“Description of Artificial Sequence: Synthetic oligonucleotide”
cccuugcagaaaguuuauccu

SEQ ID NO: 582 - RNA, 23 nt, Artificial Sequence
  source: /note=“Description of Artificial Sequence: Synthetic oligonucleotide”
aggauaaacuuucugcaagggca

SEQ ID NO: 583 - RNA, 21 nt, Artificial Sequence
  source: /note=“Description of Artificial Sequence: Synthetic oligonucleotide”
ccaauggucagaucuucaaaa

SEQ ID NO: 584 - RNA, 23 nt, Artificial Sequence
  source: /note=“Description of Artificial Sequence: Synthetic oligonucleotide”
uuuugaagaucugaccauuggcc

SEQ ID NO: 585 - RNA, 21 nt, Artificial Sequence
  source: /note=“Description of Artificial Sequence: Synthetic oligonucleotide”
caauggucagaucuucaaaau

SEQ ID NO: 586 - RNA, 23 nt, Artificial Sequence
  source: /note=“Description of Artificial Sequence: Synthetic oligonucleotide”
auuuugaagaucugaccauuggc

SEQ ID NO: 587 - RNA, 21 nt, Artificial Sequence
  source: /note=“Description of Artificial Sequence: Synthetic oligonucleotide”
aauggucagaucuucaaaaua

SEQ ID NO: 588 - RNA, 23 nt, Artificial Sequence
  source: /note=“Description of Artificial Sequence: Synthetic oligonucleotide”
uauuuugaagaucugaccauugg

SEQ ID NO: 589 - RNA, 21 nt, Artificial Sequence
  source: /note=“Description of Artificial Sequence: Synthetic oligonucleotide”
uggucagaucuucaaaauaaa

SEQ ID NO: 590 - RNA, 23 nt, Artificial Sequence
  source: /note=“Description of Artificial Sequence: Synthetic oligonucleotide”
uuuauuuugaagaucugaccauu

SEQ ID NO: 591 - RNA, 21 nt, Artificial Sequence
  source: /note=“Description of Artificial Sequence: Synthetic oligonucleotide”
uagcuaggaaguauucauuca

SEQ ID NO: 592 - RNA, 23 nt, Artificial Sequence
  source: /note=“Description of Artificial Sequence: Synthetic oligonucleotide”
ugaaugaauacuuccuagcuauu

SEQ ID NO: 593 - RNA, 21 nt, Artificial Sequence
  source: /note=“Description of Artificial Sequence: Synthetic oligonucleotide”
agcuaggaaguauucauucaa

SEQ ID NO: 594 - RNA, 23 nt, Artificial Sequence
  source: /note=“Description of Artificial Sequence: Synthetic oligonucleotide”
uugaaugaauacuuccuagcuau

SEQ ID NO: 595 - RNA, 21 nt, Artificial Sequence
  source: /note=“Description of Artificial Sequence: Synthetic oligonucleotide”
auucauucaaacuugaauauu

SEQ ID NO: 596 - RNA, 23 nt, Artificial Sequence
  source: /note=“Description of Artificial Sequence: Synthetic oligonucleotide”
aauauucaaguuugaaugaauac

SEQ ID NO: 597 - RNA, 21 nt, Artificial Sequence
  source: /note=“Description of Artificial Sequence: Synthetic oligonucleotide”
uucaaacuugaauauucuuca

SEQ ID NO: 598 - RNA, 23 nt, Artificial Sequence
  source: /note=“Description of Artificial Sequence: Synthetic oligonucleotide”
ugaagaauauucaaguuugaaug

SEQ ID NO: 599 - RNA, 21 nt, Artificial Sequence
  source: /note=“Description of Artificial Sequence: Synthetic oligonucleotide”
uugaauauucuucaaagagag

SEQ ID NO: 600 - RNA, 23 nt, Artificial Sequence
  source: /note=“Description of Artificial Sequence: Synthetic oligonucleotide”
cucucuuugaagaauauucaagu

SEQ ID NO: 601 - RNA, 21 nt, Artificial Sequence
  source: /note=“Description of Artificial Sequence: Synthetic oligonucleotide”
uugaauauucuucaaagagaa

SEQ ID NO: 602 - RNA, 23 nt, Artificial Sequence
  source: /note=“Description of Artificial Sequence: Synthetic oligonucleotide”
uucucuuugaagaauauucaagu

SEQ ID NO: 603 - RNA, 21 nt, Artificial Sequence
  source: /note=“Description of Artificial Sequence: Synthetic oligonucleotide”
gcuaauauaugugcuggcaag

SEQ ID NO: 604 - RNA, 23 nt, Artificial Sequence
  source: /note=“Description of Artificial Sequence: Synthetic oligonucleotide”
cuugccagcacauauauuagccc

SEQ ID NO: 605 - RNA, 21 nt, Artificial Sequence
  source: /note=“Description of Artificial Sequence: Synthetic oligonucleotide”
gcuaauauaugugcuggcaaa

SEQ ID NO: 606 - RNA, 23 nt, Artificial Sequence
  source: /note=“Description of Artificial Sequence: Synthetic oligonucleotide”
uuugccagcacauauauuagccc

SEQ ID NO: 607 - RNA, 21 nt, Artificial Sequence
  source: /note=“Description of Artificial Sequence: Synthetic oligonucleotide”
cauggcuguggacaguacucu

SEQ ID NO: 608 - RNA, 23 nt, Artificial Sequence
  source: /note=“Description of Artificial Sequence: Synthetic oligonucleotide”
agaguacuguccacagccauggc

SEQ ID NO: 609 - RNA, 21 nt, Artificial Sequence
  source: /note=“Description of Artificial Sequence: Synthetic oligonucleotide”
uggcuguggacaguacucugc

SEQ ID NO: 610 - RNA, 23 nt, Artificial Sequence
  source: /note=“Description of Artificial Sequence: Synthetic oligonucleotide”
gcagaguacuguccacagccaug

SEQ ID NO: 611 - RNA, 21 nt, Artificial Sequence
  source: /note=“Description of Artificial Sequence: Synthetic oligonucleotide”
uggcuguggacaguacucuga

SEQ ID NO: 612 - RNA, 23 nt, Artificial Sequence
  source: /note=“Description of Artificial Sequence: Synthetic oligonucleotide”
ucagaguacuguccacagccaug

SEQ ID NO: 613 - RNA, 21 nt, Artificial Sequence
  source: /note=“Description of Artificial Sequence: Synthetic oligonucleotide”
ggcuguggacaguacucugcu

SEQ ID NO: 614 - RNA, 23 nt, Artificial Sequence
  source: /note=“Description of Artificial Sequence: Synthetic oligonucleotide”
agcagaguacuguccacagccau

SEQ ID NO: 615 - RNA, 21 nt, Artificial Sequence
  source: /note=“Description of Artificial Sequence: Synthetic oligonucleotide”
gcuguggacaguacucugcuc

SEQ ID NO: 616 - RNA, 23 nt, Artificial Sequence
  source: /note=“Description of Artificial Sequence: Synthetic oligonucleotide”
gagcagaguacuguccacagcca

SEQ ID NO: 617 - RNA, 21 nt, Artificial Sequence
  source: /note=“Description of Artificial Sequence: Synthetic oligonucleotide”
gcuguggacaguacucugcua

SEQ ID NO: 618 - RNA, 23 nt, Artificial Sequence
  source: /note=“Description of Artificial Sequence: Synthetic oligonucleotide”
uagcagaguacuguccacagcca

SEQ ID NO: 619 - RNA, 21 nt, Artificial Sequence
  source: /note=“Description of Artificial Sequence: Synthetic oligonucleotide”
cuguggacaguacucugcuca

SEQ ID NO: 620 - RNA, 23 nt, Artificial Sequence
  source: /note=“Description of Artificial Sequence: Synthetic oligonucleotide”
ugagcagaguacuguccacagcc

SEQ ID NO: 621 - RNA, 21 nt, Artificial Sequence
  source: /note=“Description of Artificial Sequence: Synthetic oligonucleotide”
uguggacaguacucugcucaa

SEQ ID NO: 622 - RNA, 23 nt, Artificial Sequence
  source: /note=“Description of Artificial Sequence: Synthetic oligonucleotide”
uugagcagaguacuguccacagc

SEQ ID NO: 623 - RNA, 21 nt, Artificial Sequence
  source: /note=“Description of Artificial Sequence: Synthetic oligonucleotide”
guggacaguacucugcucaaa

SEQ ID NO: 624 - RNA, 23 nt, Artificial Sequence
  source: /note=“Description of Artificial Sequence: Synthetic oligonucleotide”
uuugagcagaguacuguccacag

SEQ ID NO: 625 - RNA, 21 nt, Artificial Sequence
  source: /note=“Description of Artificial Sequence: Synthetic oligonucleotide”
uggacaguacucugcucaaag

SEQ ID NO: 626 - RNA, 23 nt, Artificial Sequence
  source: /note=“Description of Artificial Sequence: Synthetic oligonucleotide”
cuuugagcagaguacuguccaca

SEQ ID NO: 627 - RNA, 21 nt, Artificial Sequence
  source: /note=“Description of Artificial Sequence: Synthetic oligonucleotide”
uggacaguacucugcucaaau

SEQ ID NO: 628 - RNA, 23 nt, Artificial Sequence
  source: /note=“Description of Artificial Sequence: Synthetic oligonucleotide”
auuugagcagaguacuguccaca

SEQ ID NO: 629 - RNA, 21 nt, Artificial Sequence
  source: /note=“Description of Artificial Sequence: Synthetic oligonucleotide”
ggacaguacucugcucaaaga

SEQ ID NO: 630 - RNA, 23 nt, Artificial Sequence
  source: /note=“Description of Artificial Sequence: Synthetic oligonucleotide”
ucuuugagcagaguacuguccac

SEQ ID NO: 631 - RNA, 21 nt, Artificial Sequence
  source: /note=“Description of Artificial Sequence: Synthetic oligonucleotide”
gacaguacucugcucaaagaa

SEQ ID NO: 632 - RNA, 23 nt, Artificial Sequence
  source: /note=“Description of Artificial Sequence: Synthetic oligonucleotide”
uucuuugagcagaguacugucca

SEQ ID NO: 633 - RNA, 21 nt, Artificial Sequence
  source: /note=“Description of Artificial Sequence: Synthetic oligonucleotide”
ugugggccaggagaaaccuua

SEQ ID NO: 634 - RNA, 23 nt, Artificial Sequence
  source: /note=“Description of Artificial Sequence: Synthetic oligonucleotide”
uaagguuucuccuggcccacaau

SEQ ID NO: 635 - RNA, 21 nt, Artificial Sequence
  source: /note=“Description of Artificial Sequence: Synthetic oligonucleotide”
ccaggagaaaccuuaucaaaa

SEQ ID NO: 636 - RNA, 23 nt, Artificial Sequence
  source: /note=“Description of Artificial Sequence: Synthetic oligonucleotide”
uuuugauaagguuucuccuggcc

SEQ ID NO: 637 - RNA, 21 nt, Artificial Sequence
  source: /note=“Description of Artificial Sequence: Synthetic oligonucleotide”
uguucuacauuaagccaauua

SEQ ID NO: 638 - RNA, 23 nt, Artificial Sequence
  source: /note=“Description of Artificial Sequence: Synthetic oligonucleotide”
uaauuggcuuaauguagaacauu

SEQ ID NO: 639 - RNA, 21 nt, Artificial Sequence
  source: /note=“Description of Artificial Sequence: Synthetic oligonucleotide”
guucuacauuaagccaauuaa

SEQ ID NO: 640 - RNA, 23 nt, Artificial Sequence
  source: /note=“Description of Artificial Sequence: Synthetic oligonucleotide”
uuaauuggcuuaauguagaacau

SEQ ID NO: 641 - RNA, 21 nt, Artificial Sequence
  source: /note=“Description of Artificial Sequence: Synthetic oligonucleotide”
uucuacauuaagccaauuaag

SEQ ID NO: 642 - RNA, 23 nt, Artificial Sequence
  source: /note=“Description of Artificial Sequence: Synthetic oligonucleotide”
cuuaauuggcuuaauguagaaca

SEQ ID NO: 643 - RNA, 21 nt, Artificial Sequence
  source: /note=“Description of Artificial Sequence: Synthetic oligonucleotide”
uucuacauuaagccaauuaaa

SEQ ID NO: 644 - RNA, 23 nt, Artificial Sequence
  source: /note=“Description of Artificial Sequence: Synthetic oligonucleotide”
uuuaauuggcuuaauguagaaca

SEQ ID NO: 645 - RNA, 21 nt, Artificial Sequence
  source: /note=“Description of Artificial Sequence: Synthetic oligonucleotide”
ucuacauuaagccaauuaagu

SEQ ID NO: 646 - RNA, 23 nt, Artificial Sequence
  source: /note=“Description of Artificial Sequence: Synthetic oligonucleotide”
acuuaauuggcuuaauguagaac

SEQ ID NO: 647 - RNA, 21 nt, Artificial Sequence
  source: /note=“Description of Artificial Sequence: Synthetic oligonucleotide”
cuacauuaagccaauuaagua

SEQ ID NO: 648 - RNA, 23 nt, Artificial Sequence
  source: /note=“Description of Artificial Sequence: Synthetic oligonucleotide”
uacuuaauuggcuuaauguagaa

SEQ ID NO: 649 - RNA, 21 nt, Artificial Sequence
  source: /note=“Description of Artificial Sequence: Synthetic oligonucleotide”
uacauuaagccaauuaaguau

SEQ ID NO: 650 - RNA, 23 nt, Artificial Sequence
  source: /note=“Description of Artificial Sequence: Synthetic oligonucleotide”
auacuuaauuggcuuaauguaga

SEQ ID NO: 651 - RNA, 21 nt, Artificial Sequence
  source: /note=“Description of Artificial Sequence: Synthetic oligonucleotide”
acauuaagccaauuaaguaua

SEQ ID NO: 652 - RNA, 23 nt, Artificial Sequence
  source: /note=“Description of Artificial Sequence: Synthetic oligonucleotide”
uauacuuaauuggcuuaauguag

SEQ ID NO: 653 - RNA, 21 nt, Artificial Sequence
  source: /note=“Description of Artificial Sequence: Synthetic oligonucleotide”
cauuaagccaauuaaguauaa

SEQ ID NO: 654 - RNA, 23 nt, Artificial Sequence
  source: /note=“Description of Artificial Sequence: Synthetic oligonucleotide”
uuauacuuaauuggcuuaaugua

SEQ ID NO: 655 - RNA, 21 nt, Artificial Sequence
  source: /note=“Description of Artificial Sequence: Synthetic oligonucleotide”
auuaagccaauuaaguauaaa

SEQ ID NO: 656 - RNA, 23 nt, Artificial Sequence
  source: /note=“Description of Artificial Sequence: Synthetic oligonucleotide”
uuuauacuuaauuggcuuaaugu

SEQ ID NO: 657 - RNA, 21 nt, Artificial Sequence
  source: /note=“Description of Artificial Sequence: Synthetic oligonucleotide”
uuaagccaauuaaguauaaag

SEQ ID NO: 658 - RNA, 23 nt, Artificial Sequence
  source: /note=“Description of Artificial Sequence: Synthetic oligonucleotide”
cuuuauacuuaauuggcuuaaug

SEQ ID NO: 659 - RNA, 21 nt, Artificial Sequence
  source: /note=“Description of Artificial Sequence: Synthetic oligonucleotide”
uuaagccaauuaaguauaaau

SEQ ID NO: 660 - RNA, 23 nt, Artificial Sequence
  source: /note=“Description of Artificial Sequence: Synthetic oligonucleotide”
auuuauacuuaauuggcuuaaug

SEQ ID NO: 661 - RNA, 21 nt, Artificial Sequence
  source: /note=“Description of Artificial Sequence: Synthetic oligonucleotide”
uaagccaauuaaguauaaagg

SEQ ID NO: 662 - RNA, 23 nt, Artificial Sequence
  source: /note=“Description of Artificial Sequence: Synthetic oligonucleotide”
ccuuuauacuuaauuggcuuaau

SEQ ID NO: 663 - RNA, 21 nt, Artificial Sequence
  source: /note=“Description of Artificial Sequence: Synthetic oligonucleotide”
uaagccaauuaaguauaaaga

SEQ ID NO: 664 - RNA, 23 nt, Artificial Sequence
  source: /note=“Description of Artificial Sequence: Synthetic oligonucleotide”
ucuuuauacuuaauuggcuuaau

SEQ ID NO: 665 - RNA, 21 nt, Artificial Sequence
  source: /note=“Description of Artificial Sequence: Synthetic oligonucleotide”
aagccaauuaaguauaaaggu

SEQ ID NO: 666 - RNA, 23 nt, Artificial Sequence
  source: /note=“Description of Artificial Sequence: Synthetic oligonucleotide”
accuuuauacuuaauuggcuuaa

SEQ ID NO: 667 - RNA, 21 nt, Artificial Sequence
  source: /note=“Description of Artificial Sequence: Synthetic oligonucleotide”
agccaauuaaguauaaagguc

SEQ ID NO: 668 - RNA, 23 nt, Artificial Sequence
  source: /note=“Description of Artificial Sequence: Synthetic oligonucleotide”
gaccuuuauacuuaauuggcuua

SEQ ID NO: 669 - RNA, 21 nt, Artificial Sequence
  source: /note=“Description of Artificial Sequence: Synthetic oligonucleotide”
agccaauuaaguauaaaggua

SEQ ID NO: 670 - RNA, 23 nt, Artificial Sequence
  source: /note=“Description of Artificial Sequence: Synthetic oligonucleotide”
uaccuuuauacuuaauuggcuua

SEQ ID NO: 671 - RNA, 21 nt, Artificial Sequence
  source: /note=“Description of Artificial Sequence: Synthetic oligonucleotide”
gcccuugcagaaaguuuaucc

SEQ ID NO: 672 - RNA, 23 nt, Artificial Sequence
  source: /note=“Description of Artificial Sequence: Synthetic oligonucleotide”
ggauaaacuuucugcaagggcaa

SEQ ID NO: 673 - RNA, 21 nt, Artificial Sequence
  source: /note=“Description of Artificial Sequence: Synthetic oligonucleotide”
gcccuugcagaaaguuuauca

SEQ ID NO: 674 - RNA, 23 nt, Artificial Sequence
  source: /note=“Description of Artificial Sequence: Synthetic oligonucleotide”
ugauaaacuuucugcaagggcaa

SEQ ID NO: 675 - RNA, 21 nt, Artificial Sequence
  source: /note=“Description of Artificial Sequence: Synthetic oligonucleotide”
cccuugcagaaaguuuauccu

SEQ ID NO: 676 - RNA, 23 nt, Artificial Sequence
  source: /note=“Description of Artificial Sequence: Synthetic oligonucleotide”
aggauaaacuuucugcaagggca

SEQ ID NO: 677 - RNA, 21 nt, Artificial Sequence
  source: /note=“Description of Artificial Sequence: Synthetic oligonucleotide”
ccaauggucagaucuucaaaa

SEQ ID NO: 678 - RNA, 23 nt, Artificial Sequence
  source: /note=“Description of Artificial Sequence: Synthetic oligonucleotide”
uuuugaagaucugaccauuggcc

SEQ ID NO: 679 - RNA, 21 nt, Artificial Sequence
  source: /note=“Description of Artificial Sequence: Synthetic oligonucleotide”
caauggucagaucuucaaaau

SEQ ID NO: 680 - RNA, 23 nt, Artificial Sequence
  source: /note=“Description of Artificial Sequence: Synthetic oligonucleotide”
auuuugaagaucugaccauuggc

SEQ ID NO: 681 - RNA, 21 nt, Artificial Sequence
  source: /note=“Description of Artificial Sequence: Synthetic oligonucleotide”
aauggucagaucuucaaaaua

SEQ ID NO: 682 - RNA, 23 nt, Artificial Sequence
  source: /note=“Description of Artificial Sequence: Synthetic oligonucleotide”
uauuuugaagaucugaccauugg

SEQ ID NO: 683 - RNA, 21 nt, Artificial Sequence
  source: /note=“Description of Artificial Sequence: Synthetic oligonucleotide”
uggucagaucuucaaaauaaa

SEQ ID NO: 684 - RNA, 23 nt, Artificial Sequence
  source: /note=“Description of Artificial Sequence: Synthetic oligonucleotide”
uuuauuuugaagaucugaccauu

SEQ ID NO: 685 - DNA, 23 bp, Artificial Sequence
  source: /note=“Description of Artificial Sequence: Synthetic primer”
gtgctggcaagtcttccaatgag

SEQ ID NO: 686 - DNA, 22 bp, Artificial Sequence
  source: /note=“Description of Artificial Sequence: Synthetic primer”
ccagtgaatggtgcgtacacag

SEQ ID NO: 687 - DNA, 22 bp, Artificial Sequence
  source: /note=“Description of Artificial Sequence: Synthetic primer”
gtctcctctgacttcaacagcg

SEQ ID NO: 688 - DNA, 22 bp, Artificial Sequence
  source: /note=“Description of Artificial Sequence: Synthetic primer”
accaccctgttgctgtagccaa